(19) United States
(12) Patent Application Publication
(10) Pub. No.: US 2022/0242833 A1
(43) Pub. Date: Aug. 4, 2022

(54) ORGANIC COMPOUND AND RED ORGANIC LIGHT EMITTING DIODE USING THE SAME

(71) Applicant: LUMINESCENCE TECHNOLOGY CORP., HSIN-CHU (TW)

(72) Inventors: TSUN-YUAN HUANG, CHIAYI (TW); CHING-YAN CHAO, HSIN-CHU (TW); FENG-WEN YEN, TAIPEI (TW)

(73) Assignee: LUMINESCENCE TECHNOLOGY CORP., HSIN-CHU (TW)

(21) Appl. No.: 17/447,695

(22) Filed: Sep. 14, 2021

(30) Foreign Application Priority Data
Jan. 29, 2021 (TW) 110103604

(51) Int. Cl. C07D 251/24; C07D 493/00; H01L 51/00; C07D 495/00; C07F 11/00

(52) U.S. Cl. CPC C07D 251/24; C07D 493/00; H01L 51/0067; H01L 51/0073; H01L 51/5096; H01L 51/0058; H01L 51/0074; C07D 495/00; C07F 11/00; H01L 51/0056

(57) ABSTRACT

An organic compound having one the following structure of formula (1) to formula (5) is described:

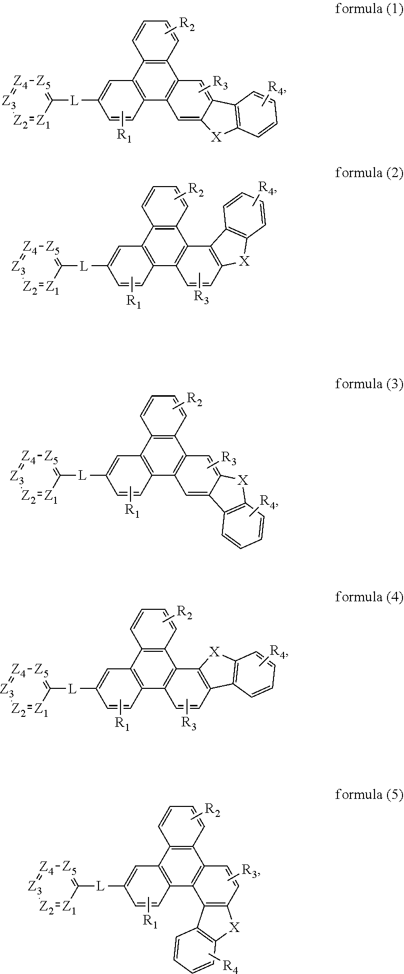

The application in a red OLED is also described.

### CROSS-REFERENCE TO RELATED APPLICATION

This application claims the benefit of priority of TW Patent Application Ser. No. 110103604 filed on Jan. 29, 2021, the content of which is incorporated herein by reference in its entirety.

### BACKGROUND

### Field of Invention

The present invention relates generally to a compound, and, more specifically, to a red organic light emitting device using the compound.

### Description of Related Art

Organic electroluminescence (organic EL) devices, i.e., organic light emitting diodes (OLEDs) are devices having planar light sources. As organic light emitting diodes have many advantages, such as self-emission, high brightness, light weight, ultra-thin profile, low power consumption, wide angle of view, high contrast, easy fabrication, and fast response time, so they can be widely used in displays, such as passive OLED (PMOLED display) and active OLED display (AMOLED display). In recent years, OLEDs have also gradually been applied to lighting-related industries, such as products of room lighting or automotive lighting recently attracted more attention.

A typical OLED is made by vacuum evaporation, in which a hole transport layer, a light emitting layer, an electron transport layer and a cathode are sequentially evaporated on a pre-patterned indium tin oxide (ITO) substrate, to form a multi-layer thin films structure. After voltage is applied to the OLED, holes and electrons are injected into the hole transport layer and the electron transport layer from the anode (ITO) and the cathode, respectively, and to form excitons in the light emitting layer. The excitons make the electrons and holes recombine to emit light in relaxation

Automotive OLED products are currently applied for red taillights of cars. For example, Audi, BMW, Benz and other major international manufacturers have launched relevant taillight applications. To use the red taillights of cars in high temperature and humidity environment, materials in an OLED product must have increased current efficiency and/or half-life. By doing so, the OLED product and the materials thereof are more suitable for use in actual automotive industry.

As used herein, “top” means furthest away from the substrate, while “bottom” means closest to the substrate. Where a second layer is described as formed on or onto a first layer, the second layer is formed further away from substrate. There may be other layers between the second layer and the first layer, unless it is specified that the second layer is “in contact with” the first layer. For example, a cathode may be described as formed onto an anode, even though there are various organic layers in between.

### SUMMARY

An aspect of the present disclosure provides an organic compound is represented by one of the following formula (1) to formula (5), or tautomers thereof:

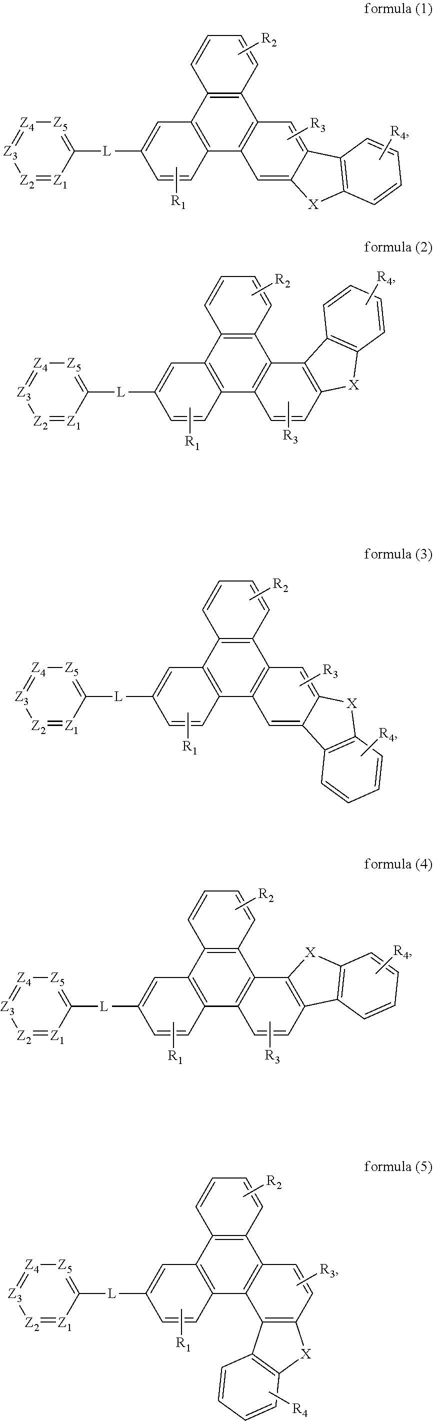

wherein X is selected from the group consisting of O, S, Se, NR₇ and SiR₅R₆;
where Z₁ to Z₅ is N or CR;
wherein at least one of Z₁ to Z₅ is N;
wherein R is selected from the group consisting of H, phenyl, pyridyl, 3-biphenyl, 2-biphenyl, 4-biphenyl, 4-(3-pyridyl)phenyl, 4-(2-pyridyl)phenyl, 4-(4-Pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof, wherein S of the dibenzothienyl group is optionally changed to Se, and wherein C connecting the dialkyl group is optionally changed to Si;
wherein L is a single bond, aryl or biphenyl;
wherein R₁ to R₇ independently represent mono to a maximum possible number of substitutions, or no substitution; and
wherein each of R₁ to R₇ is independently selected from the group consisting of halogen, alkyl, phenyl, pyridyl, 3-biphenyl, 2-biphenyl, 4-biphenyl, 4-(3-pyridinyl)phenyl, 4-(2-pyridyl) phenyl, 4-(4-pyridyl)phenyl, 3-(3-pyridyl)phenyl, 3-(2-pyridyl) phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof.

Another aspect of the present disclosure provides an organic compound represented by one of the following formula (6) to formula (10), or tautomers thereof:

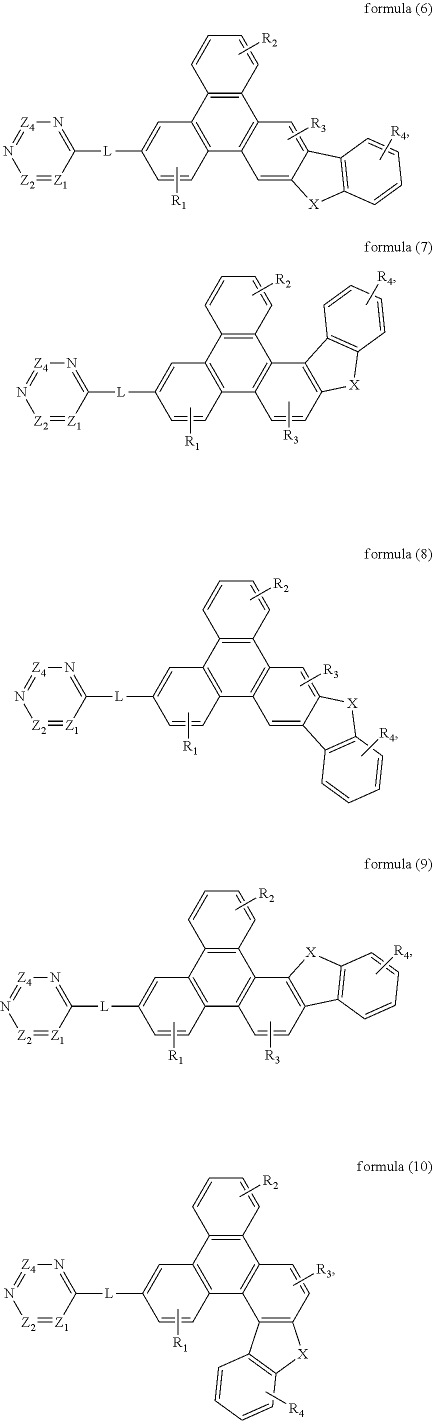

wherein X is selected from the group consisting of O, S, Se, NR₇ and SiR₅R₆;
where Z₁ is N or CR;
wherein Z₂ and Z₄ are CR;
wherein R is selected from the group consisting of H, phenyl, pyridyl, 3-biphenyl, 4-(pyridyl)phenyl, 3-(pyridyl)phenyl, 2-(pyridyl)phenyl, 9,9-dialkylfluorenyl, dibenzofuranyl, dibenzothienyl, and combinations thereof, wherein S of the dibenzothienyl group is optionally changed to Se, and wherein C connecting the dialkyl group is optionally changed to Si;
wherein L is a single bond, aryl or biphenyl;
wherein R₁ to R₇ independently represent mono to a maximum possible number of substitutions, or no substitution; and
wherein each of R₁ to R₇ is independently selected from the group consisting of halogen, alkyl, phenyl, pyridyl, 3-biphenyl, 2-biphenyl, 4-biphenyl, 4-(3-pyridinyl)phenyl, 4-(2-pyridyl) phenyl, 4-(4-pyridyl)phenyl, 3-(3-pyridyl)phenyl, 3-(2-pyridyl) phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof.

According to one embodiment of the present disclosure, X is selected from the group consisting of O, S and Se. R is selected from the group consisting of phenyl, 3-biphenyl, pyridylphenyl, 9,9-dialkylfluorenyl, dibenzofuranyl, dibenzothienyl, and combinations thereof. R₁ to R₆ independently represent mono to a maximum possible number of substitutions, or no substitution. Each of R₁ to R₆ is independently selected from the group consisting of methyl, phenyl, pyridyl, biphenyl, pyridylphenyl, 9,9-dialkylfluorenyl, dibenzofuranyl, dibenzothienyl, and combinations thereof.

According to one or more aspects of the present disclosure, X is selected from the group consisting of O, S and Se. R is selected from the group consisting of phenyl, 3-biphenyl, pyridylphenyl, 9,9-dialkylfluorenyl, dibenzofuranyl, dibenzothienyl, and combinations thereof. R₁ to R₆ independently represents mono to a maximum possible number of substitutions, or no substitution. Each of R₁ to R₆ is independently selected from the group consisting of methyl, phenyl, pyridyl, biphenyl, pyridylphenyl, 9,9-dialkylfluorenyl, dibenzofuranyl, dibenzothienyl, and combinations thereof.

According to one or more aspects of the present disclosure, X is selected from the group consisting of O, S and Se. R is selected from the group consisting of phenyl, 3-biphenyl, pyridylphenyl, 9,9-dialkylfluorenyl, dibenzofuranyl, dibenzothienyl, and combinations thereof. R₁ to R₆ independently represents mono to a maximum possible number of substitutions, or no substitution. Each of R₁ to R₆ is independently selected from the group consisting of methyl, phenyl, pyridyl, biphenyl, pyridylphenyl, 9,9-dialkylfluorenyl, dibenzofuranyl, dibenzothienyl, and combinations thereof.

According to one or more aspects of the present disclosure, one of the following is true:
only one of Rs is 3-biphenyl if X represents O;
R is phenyl if X represents CR₅R₆;
X represents O or CR₅R₆ if R is phenyl, wherein R₅ and R₆ are methyl; and
at least one of Rs is phenyl, and the other R is selected from the group consisting of phenyl, pyridyl, biphenyl, pyridylphenyl, 9,9-dialkylfluorenyl, dibenzofuranyl, dibenzothienyl, and combinations thereof if X represents CR₅R₆, and if R₅ and R₆ are methyl.

According to one or more aspects of the present disclosure, a red organic light emitting diode comprises a first electrode, a second electrode, and an organic thin film layer between the first electrode and the second electrode. The organic thin film layer is selected from the group consisting of a first light emitting layer, a first hole blocking layer, a first electron transport layer, and combinations thereof. The first light emitting layer comprises a first host and a first guest. The organic thin film layer comprises the organic compound of the present disclosure. An electroluminescence (EL) peak of the first guest is greater than about 600 nm.

According to one or more aspects of the present disclosure, the first host comprises the organic compound as a first material. The first hole blocking layer comprises the organic compound as a second material. The second material is the same as the first material.

According to one or more aspects of the present disclosure, the red organic light emitting diode further comprises a second light emitting layer and a second electron transport layer. The second light emitting layer comprises a second host. The second host comprises the organic compound as a third material, and wherein the third material is the same as the first material.

According to one or more aspects of the present disclosure, the red organic light emitting diode further comprises a second hole blocking layer.

According to one or more aspects of the present disclosure, the second hole blocking layer comprises the organic compound as a fourth material. The fourth material is the same as the first material.

### BRIEF DESCRIPTION OF THE DRAWINGS

FIG. 1 is a schematic view showing a first embodiment of the present disclosure.

FIG. 2 is a schematic view showing a second embodiment of the present disclosure.

FIG. 3 is a schematic view showing a third embodiment of the present disclosure.

FIG. 4 is a schematic view showing a fourth embodiment of the present disclosure.

### DETAILED DESCRIPTION

Plural embodiments of the present disclosure are disclosed through drawings. For the purpose of clear illustration, many practical details will be illustrated along with the description below. It should be understood that, however, these practical details should not limit the present disclosure. In other words, in embodiments of the present disclosure, these practical details are not necessary. In addition, for the purpose of simplifying the drawings, some conventional structures and components are simply and schematically depicted in the figures.

Aiming at a gap or a specific layer calibrated by the receptor component, to research and develop new organic compounds having functions of OLED photoelectric mechanism and actually solving technical problems, is not only one of the main ideas and approaches in the research and development, but also key technical basis for filing patent applications. The above-mentioned research methods of photoelectric mechanism have become the key work for the research and development of organic compounds in this field. Therefore, it is not suitable to deviate from the research methods in this field when determining the technical problems actually solved by the present disclosure. When the concept of the present disclosure is analyzed, it will help to more accurately determine the technical problems actually solved by the compound invention, and to lay a solid foundation for the judgment of technical enlightenment, if the above-mentioned research methods of those skilled in the art is followed, if the process of inventing an organic compound and a red OLED using the same is experienced, and if the applicant's technical contributions are essentially grasped.

It is to be understood that although particular phrases used herein, such as “first”, “second”, “third”, and so on, are used to describe different components, members, regions, layers, and/or sections, these components, members, regions, layers, and/or sections should not be limited by these terms. These phrases are used to distinguish one component, member, region, layer, or section from another component, member, region, layer, or section. In this way, a first component, member, region, layer, and/or section to be described below may be referred to as a second component, member, region, layer, and/or section, without departing from the spirit and scope of the present disclosure.

Spatially relative phrases, such as “onto”, “on”, “under”, “below”, “underlying”, “beneath”, “above”, and so on used herein, are used for facilitating description of a relation between one component or feature and another component or feature depicted in the drawings. Therefore, it can be understood that, in addition to directions depicted in the drawings, the spatially relative terms mean to include all different orientations during usage or operations of the device. For example, it is assumed that a device in a figure is reversed upside down, a component described as being “under”, “below”, or “beneath” another component or feature is oriented “onto” or “on” the other component or feature. Therefore, these exemplary terms “under” and “below” may include orientations above and below. The device may be otherwise oriented (e.g., turned by 90 degrees, or other orientations), and the spatially relative terms used herein should be explained accordingly.

Accordingly, it may be understood that when a component or a layer is referred to as being “onto”, “on”, “connected to”, or “coupled to” another component or another layer, it may be immediately on the other component or layer, or connected to or coupled to the other component or layer, or there may be one or more intermediate components or intermediate layers. Further, it can be understood that when a component or a layer is referred to as being “between” two components or two layers, it may be the only component or layer between the two components or layers, or there may be one or more intermediate components or intermediate layers.

Terminologies used herein are only for the purpose of describing particular embodiments, but not limiting the present disclosure. The singular form of “a” and “the” used herein may also include the plural form, unless otherwise indicated in the context. Accordingly, it can be understood that when there terms “include” or “comprise” are used in the specification, it clearly illustrates the existence of a specified feature, bulk, step, operation, component, and/or member, while not excluding the existence or addition of one or more features, bulks, steps, operations, components, members and/or groups thereof. “And/or” used herein includes any and all combinations of one or more related terms that are listed. When a leading word, such as “at least one of”, is added ahead of a component list, it is to describe the entire component list, but not individual components among the list.

It is to be understood that when a molecular fragment is described as being a substituent or otherwise attached to another moiety, its name may be written as if it were a fragment (e.g., dibenzofuranyl, naphthyl, biphenyl, arylene, phenyl, phenylene) or as if it were the whole molecule (e.g., dibenzofuran, naphthalene, biphenyl, aromatic group, benzene). As used herein, these different ways of designating a substituent or attached fragment are considered to be equivalent.

The terms “substituted” and “substitution” refer to a substituent bonded to the relevant position, e.g., a carbon or nitrogen. Accordingly, for example, when R₁ represents mono substitution, one R₁ must not be H. Similarly, when R₁ represents at least di substitutions, at least two R₁ must not be H. In addition, when R₁ represents no substitution, R₁, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine.

As used herein, a maximum possible number of substitutions in a ring structure will depend on the total number of available valencies in the ring atoms. See

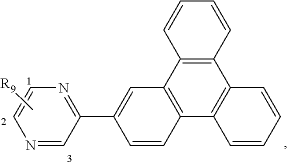

for example, there are three available valencies in the ring atoms of the left heteroaryl ring, so the maximum possible number of substitutions of R₉ substitution is 3.

As used herein, when a single ring of a compound's formula is substituted a non-symmetrical polycyclic aryl, the formula may represent at least two compounds. For example, the formula

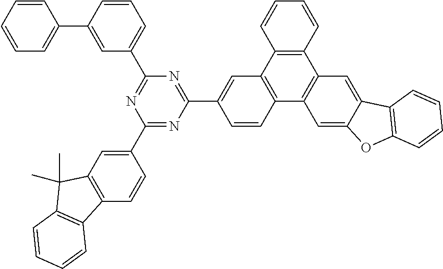 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

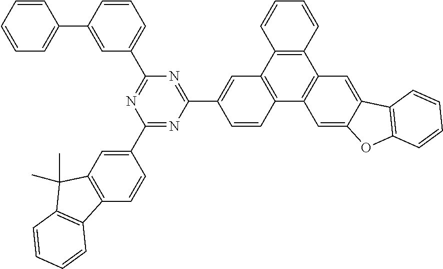 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

and represents a second compound

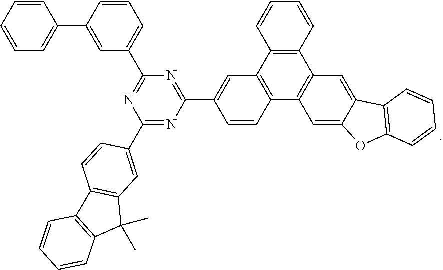 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

Even if the scope of the patent application only draws the first compound, it is not limited to it.

That is, the formula of the first compound is to further comprises the second compound. See the following for reference.

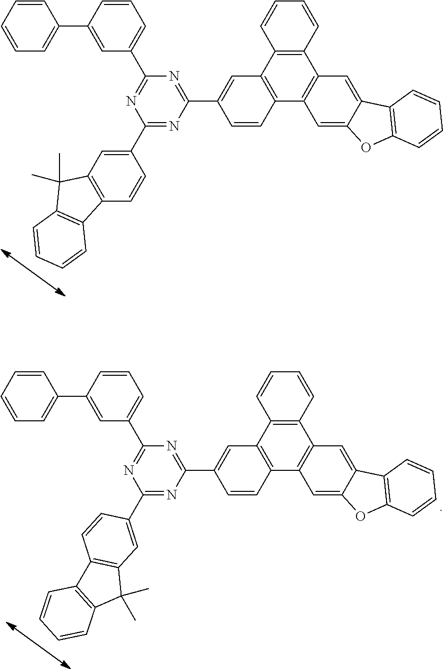 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21 ; CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

As used herein, the terms “R₁”, “R₂”, “R₃”, “R₄”, “R₅”, “R₆” . . . or “R₇” may independently be hydrogen or a substituent selected from the group consisting of aryl, alkyl, alkylphenyl, halogen, phenyl, methylphenyl, pyridyl, biphenyl, pyridylphenyl, 9,9-dialkylfluorenyl, dibenzofuranyl, two Benzothienyl, carbazolyl, methyl, butyl, n-butyl, hexyl, propyl, isopropyl, hexylphenyl, triazinyl, diazinyl, naphthyl, heteroaryl, aralkyl, Trifluoromethyl, cyano, nitro, trimethylsilyl, silyl, aryl substituted or unsubstituted by methyl or hexyl, biphenyl, pyridylphenyl, m-terphenyl, diiso Butylcarbazolyl, phenylcarbazolyl, dimethylcarbazolyl, cyano, phenyl, dicyanophenyl, nitro, cycloalkyl, heterocycloalkyl, aromatic group, aromatic amine Group, heteroarylamine group, deuterium, alkoxy, amino, silyl, fluorenyl, benzofluorenyl, anthracenyl, phenanthryl, pyrenyl, alkenyl, cycloalkenyl, heteroalkenyl, alkynyl, carbonyl, Carboxylic acid, ether, ester, glycol, isonitrile, thio, sulfinamide, sulfonyl, phosphoric acid, triphenylene, benzimidazole, dicarbazolyl, diphenylphosphine oxide, Phenanthroline group, dihydroacridinyl, phenothiazinyl, phenoxazinyl, dihydrophenazinyl, diphenylamino, triphenylamino, phenyldibenzofuranylamino, phenyldiphenyl Thioanilino, and combinations thereof. In addition, when each of R, R₁, R₂, R₃, R₄, R₅, R₆ . . . or R_{N} represents a substituent, two adjacent substituents, for example, two adjacent R substituents may be optionally bonded (connected) or fused together to form a single ring structure (such as benzene), or to form fused rings (also polycyclic aromatic groups such as naphthalene. The fused rings are formed together with the substituted.

As used herein, if the term “a first integer to a second integer” is used to express a plurality of solutions, it may cover the first integer, the second integer, and each integer between the first and the second integers. That is to say, when the term “a first integer to a second integer” expresses a plurality of solutions, all of its integers are parallel technical solutions. In this case, the term “a first integer to a second integer” is not used to express a numerical range. For example, 1 to 4 covers 1, 2, 3, 4 and does not include 1.5. For another example, 0 to 3 cover 0, 1, 2, and 3, wherein 0, 1, 2, and 3 are technical solutions in parallel. For another example, 1 to 5 covers 1, 2, 3, 4, and 5, among which 1, 2, 3, 4, and 5 are parallel technical solutions. These solutions may be, for example, the number of substituents or the number of carbon atoms. It is noted that “a maximum possible number of substitutions” is also a kind of integer.

As used herein, “combinations thereof” indicates that one or more members of the applicable list are combined to form a known or chemically stable arrangement that one of ordinary skill in the art can envision from the applicable list. For example, two phenyl groups can be combined (bonded) to form a biphenyl group. If a methyl phenyl group substituted for a first C is combined to a methyl group substituted for a second C adjacent to the first C, a naphthalene is formed (together with the adjacent Cs). The monocyclic aromatic group and the polycyclic aromatic group can be bonded (connected) together through a direct bond (single bond), or can be condensed to form two adjoining rings in which two carbons are common the two adjoining rings. Alkyl and deuterium can be combined to form partially or fully deuterated alkyl. Halogen and alkyl can be combined to form haloalkyl. Halogen, alkyl and aryl can be combined to form haloaralkyl.

The following description of various terms related to substituents, one of the intentions, is to show that some substituents are mutually replaceable and/or have common functions. In other words, in the field of the present disclosure, these substituents may be of the same category.

The term “alkyl” refers to and includes both straight and branched chain alkyl radicals. Preferred alkyl groups are those containing 30 or fewer carbon atoms, preferably 1 to 20 carbon atoms, more preferably 1 to 15 carbon atoms, and most preferably 1 to 12 carbon atoms. Suitable alkyl groups include methyl, ethyl, propyl, isopropyl, butyl, n-butyl, hexyl, n-hexyl, 1-methylethyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “aryl” or “aromatic group” as used herein are interchangeable with each other and contemplates monocyclic aromatic groups (or hydrocarbyls), polycyclic aromatic groups (or hydrocarbyls), fused ring hydrocarbon units, and combinations thereof. The polycyclic aromatic group may have two, three, four, five, or more rings in which two carbons are common to two adjoining rings (meaning that the two adjacent rings are “fused”). A polycyclic aromatic group can be named a bicyclic aromatic group if it has two rings; if it has three rings, it can be named a tricyclic aromatic group, and so on. In a polycyclic aromatic group, at least one of the polycyclic rings is an aromatic group, e.g., the other rings can be cycloalkyls, cycloalkenyls, aryl, heterocycles, and/or heteroaryls. In terms of the number of carbon atoms, preferred aromatic groups are those containing 30 or fewer carbon atoms, preferably 6 to 30 carbon atoms, more preferably 6 to 20 carbon atoms, and most preferably 6, 10 or 12 carbon atoms.

Suitable aromatic groups include but not limited to phenyl, 3-biphenyl, 2-biphenyl, 4-biphenyl, fluorenyl, fluoranthene, benzanthracene, benzo[c]phenanthrene, benzofluorene, 9,9-dialkylfluorenyl, 9,9-dimethylfluorene, naphthalene, phenanthrene, anthracene, triphenylene, pyrene, phenanthrene, perylene, terphenyl, terphenyl, m-terphenyl, p-terphenyl, o-terphenyl, tetraphenyl, phenalene, 9H-fluorene, and azulene. Among the suitable aromatic groups, preferred include phenyl, 3-biphenyl, 2-biphenyl, 4-biphenyl, fluorenyl, fluoranthene, benzanthracene, benzo[c]phenanthrene, benzofluorene, 9,9-dimethylfluorene Base, naphthalene, phenanthrene, anthracene, triphenylene, pyrene, chrysene, perylene, terphenyl, fluorene and naphthalene. In addition, the above-mentioned “aryl” or “aromatic group” may be optionally substituted, for example, the two hydrogen atoms commonly bonded on the same carbon atom of benzofluorene may be further substituted with two methyl groups, which product may be named dimethyl-benzofluorene. As another example, the phenyl group may be further substituted with methyl, hexyl or pyridyl.

It is noted that a monocyclic azaphenyl group (pyridyl), or a monocyclic azaphenyl group (pyrimidinyl or triazinyl) containing two or more nitrogen atoms that are not adjacent to each other on the ring:

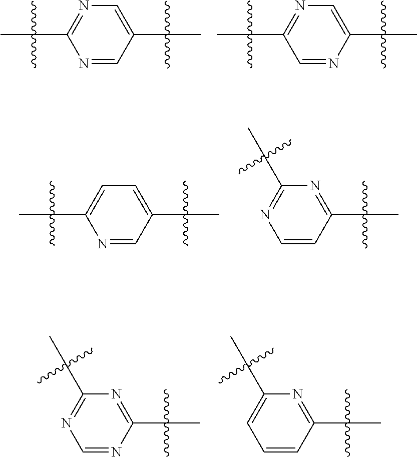 [SMILES: *c1cnc(*)nc1 ; *c1cnc(*)cn1 ; *c1ccc(*)nc1 ; *c1ccnc(*)n1 ; *c1ncnc(*)n1 ; *c1cccc(*)n1]

Like phenylene, they have three pairs of π electrons that resonate with each other and have similar chemical properties. Therefore, if the compound preferably has one of the substituents, other substituents may also be preferred.

As used herein, the terms “heteroaryl” or “heteroaromatic group” are interchangeable with each other. “Heteroaryl” or “heteroaromatic group” may be selected from the group consisting of a monocyclic heteroaromatic group containing one, two, three, four, five or more heteroatoms, a polycyclic heteroaromatic group having two or more rings, and combinations thereof. Heteroatoms include, but are not limited to, O, S, N, P, B, Si and Se. In many cases, O, S, N, Si and Se is the preferred heteroatom. The “monocyclic heteroaromatic group” is preferably a single ring having 5 or 6 ring atoms, and the ring may have one to six heteroatoms. A “polycyclic heteroaromatic group” may have two, three, four, five, six or more rings, where two carbons are common to two adjoining rings (meaning that the rings are “fused”). A polycyclic heteroaromatic group can be named a bicyclic heteroaromatic group if it has two rings; if it has three rings, it can be named a tricyclic heteroaromatic group, and so on. In a polycyclic heteroaromatic group, at least one of the rings is a heteroaryl, e.g., the other rings can be cycloalkyls, cycloalkenyls, aryl, heterocycles, and/or heteroaryls. In terms of the number of carbon atoms, the polycyclic heteroaromatic groups can have from one to six heteroatoms per ring of the polycyclic heteroaromatic groups. Preferred heteroaryl groups are those containing 30 or fewer carbon atoms, preferably 3 to 30 carbon atoms, more preferably 3 to 20 carbon atoms, and most preferably 3 to 12 carbon atoms. Suitable heteroaromatic groups may include dibenzothienyl, dibenzofuranyl, carbazolyl, pyridyl, triazinyl, diazinyl, 1,3,5-triazinyl, quinazoline, quinoxaline, benzoquinazoline, Pyrimidine, dibenzoselenophene, furan, thiophene, benzofuran, benzothiophene, benzoselenophene, indolocarbazole, pyridylindole, pyrrolodipyridine, pyrazole, imidazole, triazole, Oxazole, thiazole, oxadiazole, oxtriazole, dioxazole, thiadiazole, pyridazine, pyrazine, oxazine, oxthiazine, oxadiazine, indole, benzimidazole, indazole, inoxa Oxazine, benzoxazole, benzisoxazole, benzothiazole, quinoline, isoquinoline, cinnoline, naphthyridine, phthalazine, pteridine, xanthene, acridine, phenazine, phenazine thiazine, phenoxazine, benzofuropyridine, furobipyridine, benzothienopyridine, thienodipyridine, benzoselenophenopyridine and selenophenopyridine, 1,2-azaborane, 1,3-azaborane, 1,4-azaborane, borazine, and other nitrogen analogs of the aza-derivatives described above. Among the suitable heteroaromatic groups, preferred include dibenzothiophene, dibenzofuran, carbazole, pyridine, quinazoline, quinoxaline, benzoquinazoline, dibenzoselenophene, indolocarbazole, imidazole, triazine, benzimidazole, 1,2-azaborane, 1,3-azaborane, 1,4-azaborane, borazine, and other nitrogen analogs of the aza-derivatives described above. In addition, the “heteroaryl” or “heteroaromatic group” may be optionally substituted. For example, the carbazolyl group can be further substituted with two isobutyl groups, which is called diisobutylcarbazole.

It is noted that if the compounds drawn in this article, whether in the specification or the scope of the patent application, contain dibenzofuran, the substantial meaning of the drawn dibenzofuran is sufficient to comprise polycyclic heteroaromatic groups containing dibenzothiophene. The O of dibenzofuran and S of dibenzothiophene are elements of the same group. The structure and chemical properties of O and S are very similar, and they may therefore be used instead of each other. For example, even if the scope of the patent application only draws the polycyclic heteroaromatic compound containing dibenzofuran on the left side of the figure, it is not limited to this. In addition, the S of the dibenzothienyl group has to be changed to Se for similar reasons, because Se and S are elements of the same group, with very similar structures and chemical properties, and can be used instead of each other.

If the C of the dialkyl group of the 9,9-dialkylfluorenyl group is changed to Si, the changed group is not for defining a polycyclic aromatic group, but a polycyclic heteroaromatic group. Broadly speaking, Si and C are elements of the same family, their structure and chemical properties are very similar, and they can therefore be used instead of each other. Accordingly, changing the C of the dialkyl group of the 9,9-dialkylfluorenyl group to Si is a suitable example. In addition, the alkyl group attached to Si can also be changed to Rs, and each Rs can be the same or different alkyl groups or other substituents. That is, each Rs can be H (hydrogen) or a substituent selected from the group consisting of deuterium, halogen, alkyl, cycloalkyl, heteroalkyl, heterocycloalkyl, aralkyl, alkoxy, aryloxy, amino, silyl, alkenyl, cycloalkenyl, heteroalkenyl, alkynyl, aryl, heteroaryl, and combinations thereof. Preferred Rs is selected from the group consisting of alkyl, aryl, tolyl, pyridyl, hexylphenyl, naphthyl, and combinations thereof.

Among the listed heteroaromatic groups, the “aza” designation in the fragments i.e., aza-dibenzofuran, aza-dibenzothiophene, etc. means that one or more of the C—H groups in the respective fragment can be replaced by a nitrogen atom, for example, and without any limitation, azatriphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

Among the aryl and heteroaryl groups listed above, aryl, alkyl, alkylphenyl, halogen, phenyl, methylphenyl, pyridyl, biphenyl, pyridylphenyl, 9,9-dialkylfluorenyl, dibenzofuranyl, dibenzothienyl, dibenzoselenophene, carbazolyl, methyl, ethyl, isopropyl, n-butyl, n-hexyl, triazine, Pyrimidine, quinoxaline, quinazoline, benzoquinazoline, pyrene, perylene, naphthalene, anthracene, triphenylene, fluoranthene, dimethyl-benzofluorene, phenanthrene, phenanthrene, benzo[c]phenanthrene, benzanthracene, indolocarbazole, imidazole, pyrazine, and benzimidazole, as well as the aza-derivatives or analogs thereof, are of particular interest.

The terms alkyl, heteroaryl, aryl, and heterocyclic group, as used herein, are independently unsubstituted, or independently substituted, with one or more general substituents selected from the group consisting of methyl, ethyl, propyl, butyl, hexyl, halogen, alkyl, phenyl, methylphenyl, pyridyl, biphenyl, pyridylphenyl, 9,9-dialkylfluorenyl, two benzofuranyl, dibenzothienyl, dibenzoselenophene, amino, silyl, cyano, trifluoromethyl, cyanobenzene, dicyanobenzene, benzofluorene, deuterium, halogen, alkyl, Cycloalkyl, heteroalkyl, aryl, aralkyl, alkoxy, aryloxy, cycloamino, alkenyl, cycloalkenyl, heteroalkenyl, alkynyl, heteroaryl, acyl, carbonyl, Carboxylic acid, ether, ester, nitrile, isonitrile, thio group, sulfinyl group, sulfinyl group, phosphine group, and combinations thereof.

The terms “halogen” and “halide” are used interchangeably and refer to fluorine, chlorine, bromine, or iodine. The term “trifluoromethyl” refers to a —CF₃ radical. The term “cyano” refers to a —C═N radical. The term “nitro” refers to a —NO₂ radical.

As used herein, abbreviations refer to materials and/or films as follows:

LiQ: 8-hydroxyquinolato-lithium
Host1: a first host (comprising a first material)
Host2: a second host (comprising a second material)
HAT-CN: Dipyrazino[2,3-f:2,3-]quinoxaline-2,3,6,7,10,11-hexacarbonitrile
EIL: electron injecting layer
ETL: electron transport layer
ETM: electron transport material
EML: emissive layer
EBL: electron blocking layer
HTL: hole transporting layer
HIL: hole injection layer
ITO: indium tin oxide
EL: electroluminescence
HBL: hole blocking layer

As used herein, some of the material structures used in Comparative Example 1 and Comparative Example 2 are as follows:

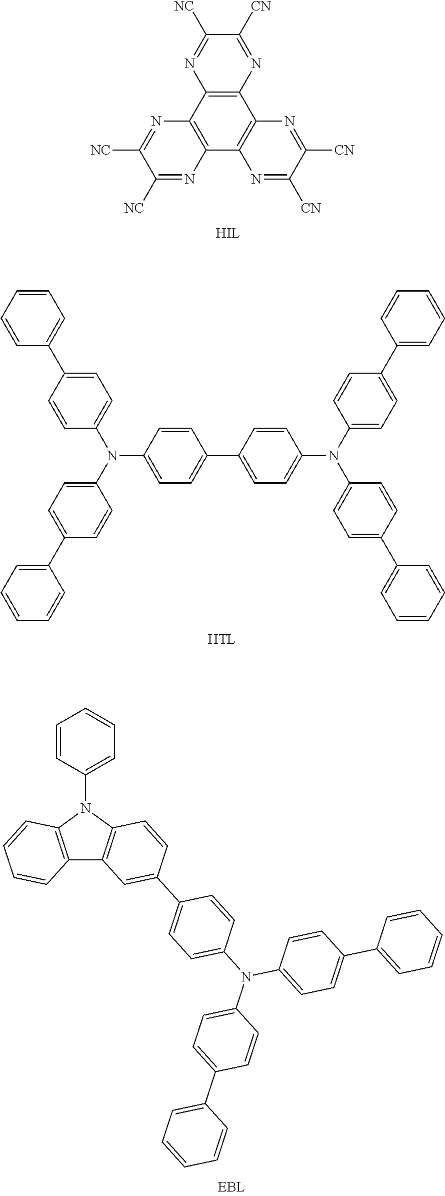 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

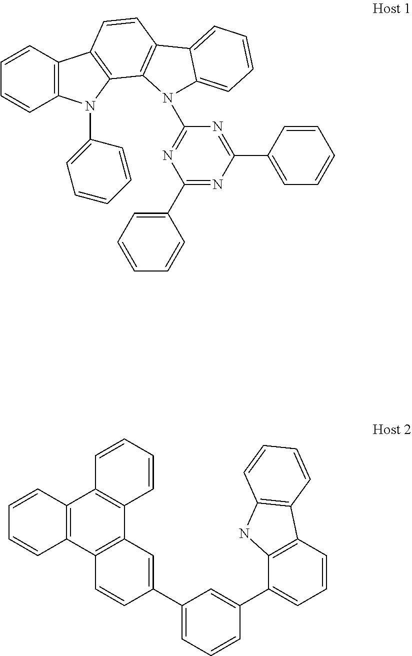 [SMILES: c1ccc(-c2nc(-c3ccccc3)nc(-n3c4ccccc4c4ccc5c6ccccc6n(-c6ccccc6)c5c43)n2)cc1 ; c1cc(-c2ccc3c4ccccc4c4ccccc4c3c2)cc(-c2cccc3c2[nH]c2ccccc23)c1]

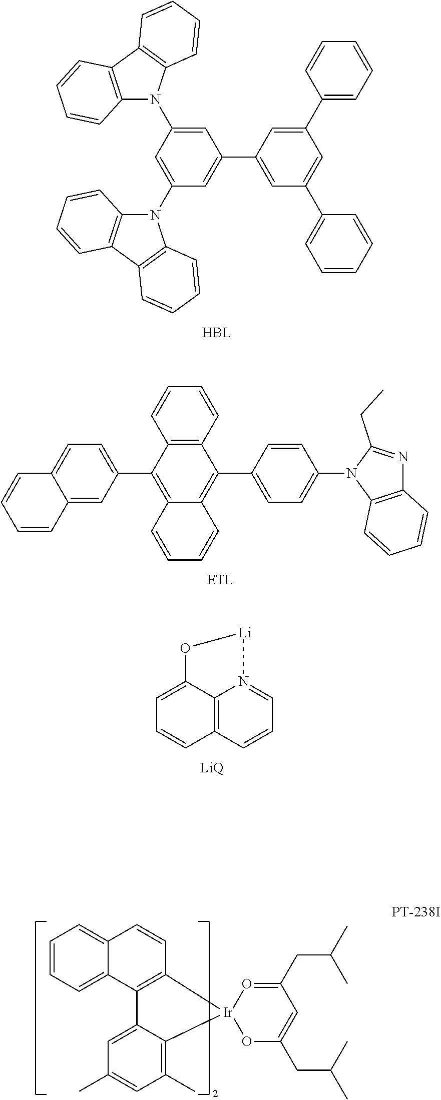

In the present disclosure, after producing a red OLED, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

In some embodiments, an organic compound is represented by one of the following formula (1) to formula (5), or tautomers thereof:

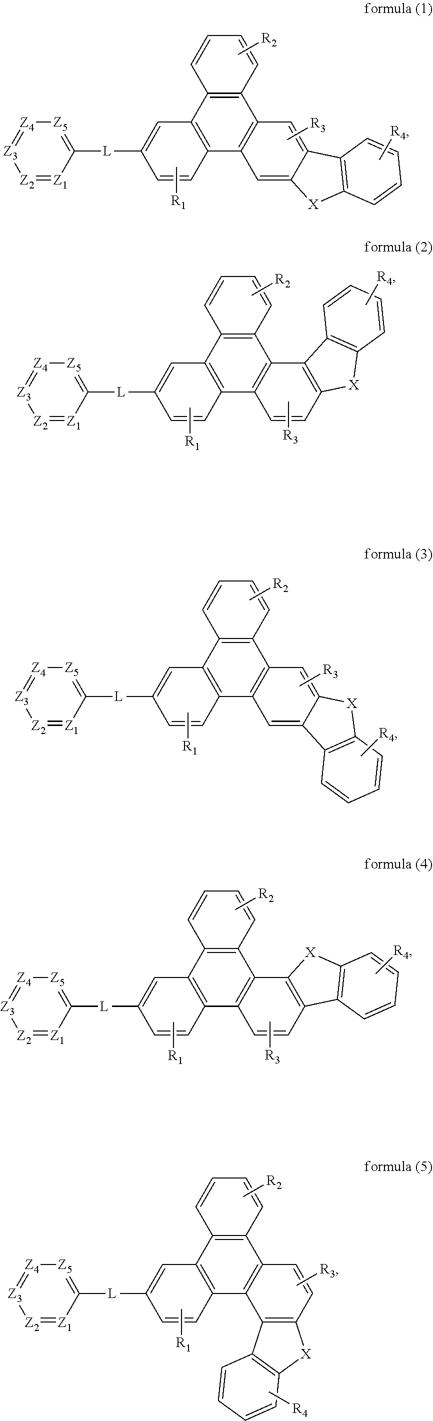

wherein X is selected from the group consisting of O, S, Se, NR₇ and SiR₅R₆;
where Z₁ to Z₅ is N or CR;
wherein at least one of Z₁ to Z₅ is N;
wherein R is selected from the group consisting of H, phenyl, pyridyl, 3-biphenyl, 2-biphenyl, 4-biphenyl, 4-(3-pyridyl)phenyl, 4-(2-pyridyl)phenyl, 4-(4-Pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof, wherein the S of the dibenzothienyl group in R is optionally changed to Se, and wherein the C connecting the dialkyl group in R is optionally changed to Si;
wherein L is a single bond, aryl or biphenyl;
wherein R₁ to R₇ independently represent mono to a maximum possible number of substitutions, or no substitution; and
wherein each of R₁ to R₇ is independently selected from the group consisting of halogen, alkyl, phenyl, pyridyl, 3-biphenyl, 2-biphenyl, 4-biphenyl, 4-(3-pyridinyl)phenyl, 4-(2-pyridyl) phenyl, 4-(4-pyridyl)phenyl, 3-(3-pyridyl)phenyl, 3-(2-pyridyl) phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof.

In many cases, an OLED can be considered to be composed of acceptor component and a donor organic compound. In the receptor component of the OLED, a plurality of organic layers may respectively have different thickness, may comprise different organic compounds as materials, may be formed in different sequences. The organic layers are characterized by the differences, richness or diversity, those receptor components may therefore have different formulas, activity, and functions.

In addition, various acceptor components of OLEDs are highly selective when an organic compound (donor) is applied. Even the same organic compound may have completely different performance in different acceptor components. Furthermore, it should be considered that the mechanisms of reaction between different acceptor components and donor organic compounds have specificity and complexity. If only the name of an organic layer of the acceptor is disclosed, or is just given a simple text hint, it is not sufficient to enforce the organic layer and solve key technical problems. It can only, at best, provide directions for subsequent development.

In some embodiments, an organic compound represented by one of the following formula (6) to formula (10), or tautomers thereof:

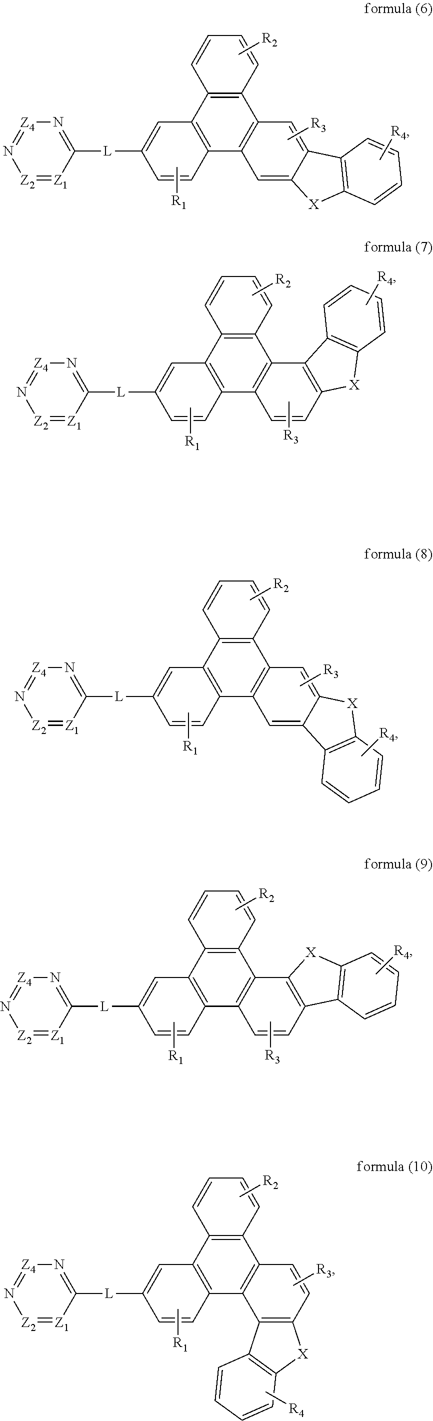

wherein X is selected from the group consisting of O, S, Se, NR₇ and SiR₅R₆;
where Z₁ is N or CR;
wherein Z₂ and Z₄ are CR;
wherein R is selected from the group consisting of H, phenyl, pyridyl, 3-biphenyl, 4-(pyridyl)phenyl, 3-(pyridyl)phenyl, 2-(pyridyl)phenyl, 9,9-dialkylfluorenyl, dibenzofuranyl, dibenzothienyl, and combinations thereof, wherein the S of the dibenzothienyl group in R is optionally changed to Se, and wherein the C connecting the dialkyl group in R is optionally changed to Si;
wherein L is a single bond, aryl or biphenyl;
wherein R₁ to R₇ independently represent mono to a maximum possible number of substitutions, or no substitution; and
wherein each of R₁ to R₇ is independently selected from the group consisting of halogen, alkyl, phenyl, pyridyl, 3-biphenyl, 2-biphenyl, 4-biphenyl, 4-(3-pyridinyl)phenyl, 4-(2-pyridyl) phenyl, 4-(4-pyridyl)phenyl, 3-(3-pyridyl)phenyl, 3-(2-pyridyl) phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof.

In some embodiments, an organic compound represented by one of the following formula (6) to formula (10), or tautomers thereof:

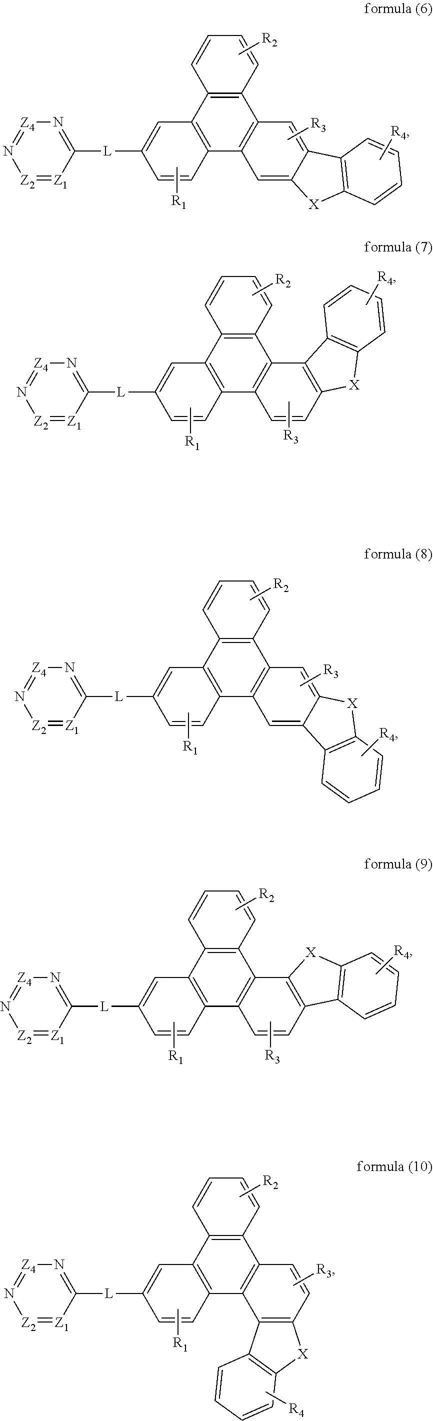

wherein X is selected from the group consisting of O, S, Se and SiR₅R₆;
where Z₁ is N or CR;
wherein Z₂ and Z₄ are CR;
wherein R is selected from the group consisting of H, phenyl, pyridyl, 3-biphenyl, 4-(pyridyl)phenyl, 3-(pyridyl)phenyl, 2-(pyridyl)phenyl, 9,9-dialkylfluorenyl, dibenzofuranyl, dibenzothienyl, and combinations thereof, wherein the S of the dibenzothienyl group in R is optionally changed to Se, and wherein the C connecting the dialkyl group in R is optionally changed to Si;
wherein L is a single bond, aryl or biphenyl; and
wherein each of R₁ to R₆ is independently selected from the group consisting of methyl, phenyl, pyridyl, biphenyl, pyridylphenyl, 9,9-dialkylfluorenyl, dibenzofuranyl, dibenzothienyl, and combinations thereof.

According to one or more aspects of the present disclosure, one of the following is true:
only one of Rs is 3-biphenyl if X represents O;
R is phenyl if X represents CR₅R₆;
X represents O or CR₅R₆ if R is phenyl, wherein R₅ and R₆ are methyl; and
at least one of Rs is phenyl, and the other R is selected from the group consisting of phenyl, pyridyl, biphenyl, pyridylphenyl, 9,9-dialkylfluorenyl, dibenzofuranyl, dibenzothienyl, and combinations thereof if X represents CR₅R₆, and if R₅ and R₆ are methyl.

In some embodiments, an organic compound is selected from the group consisting of the following compounds C1 to C150:

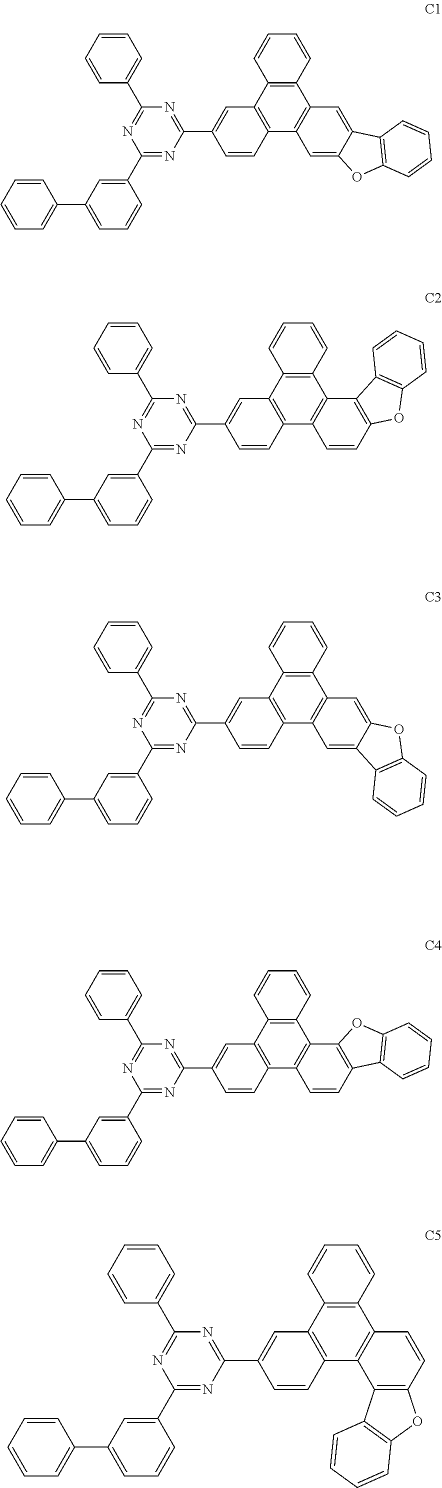 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; c1ccc(-c2cccc(-c3nc(-c4ccccc4)nc(-c4ccc5c(c4)c4ccccc4c4cc6oc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6oc54)n3)c2)cc1 ; c1ccc(-c2cccc(-c3nc(-c4ccccc4)nc(-c4ccc5c(c4)c4ccccc4c4ccc6oc7ccccc7c6c45)n3)c2)cc1]

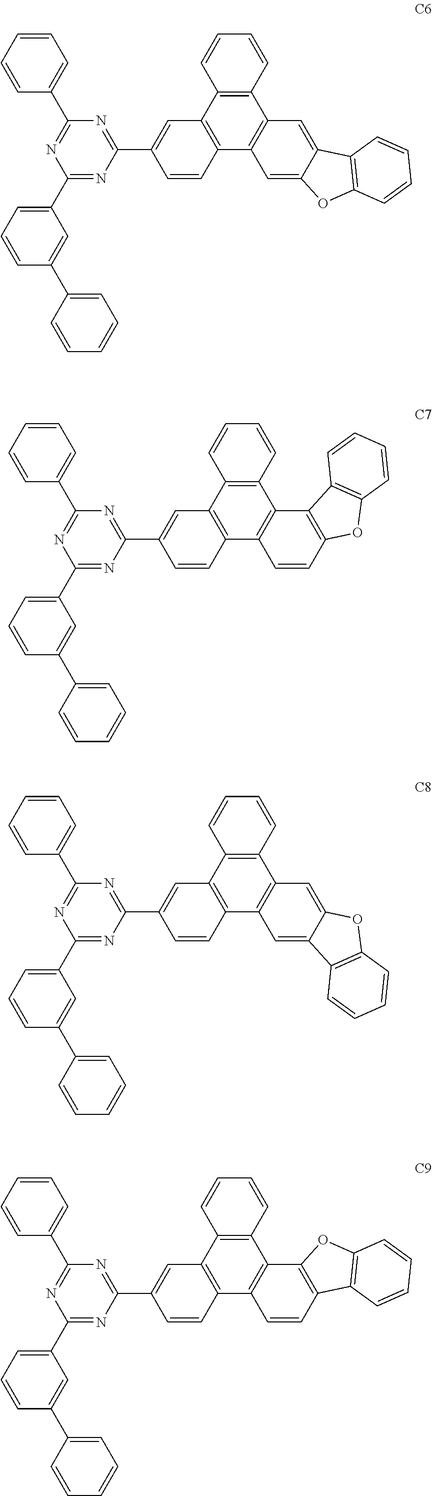 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; c1ccc(-c2cccc(-c3nc(-c4ccccc4)nc(-c4ccc5c(c4)c4ccccc4c4cc6oc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6oc54)n3)c2)cc1]

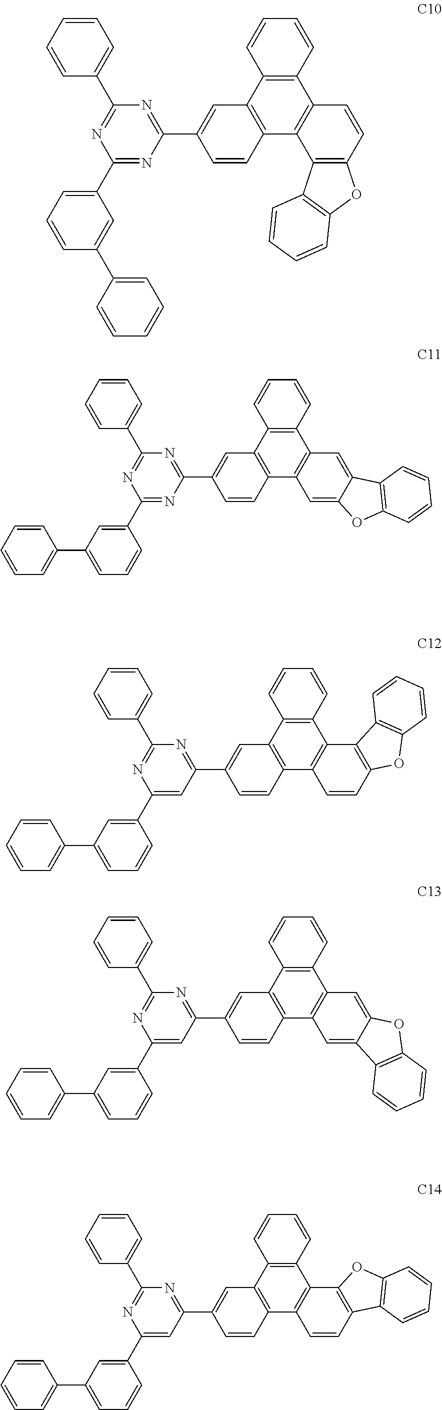 [SMILES: c1ccc(-c2cccc(-c3nc(-c4ccccc4)nc(-c4ccc5c(c4)c4ccccc4c4ccc6oc7ccccc7c6c45)n3)c2)cc1 ; c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1]

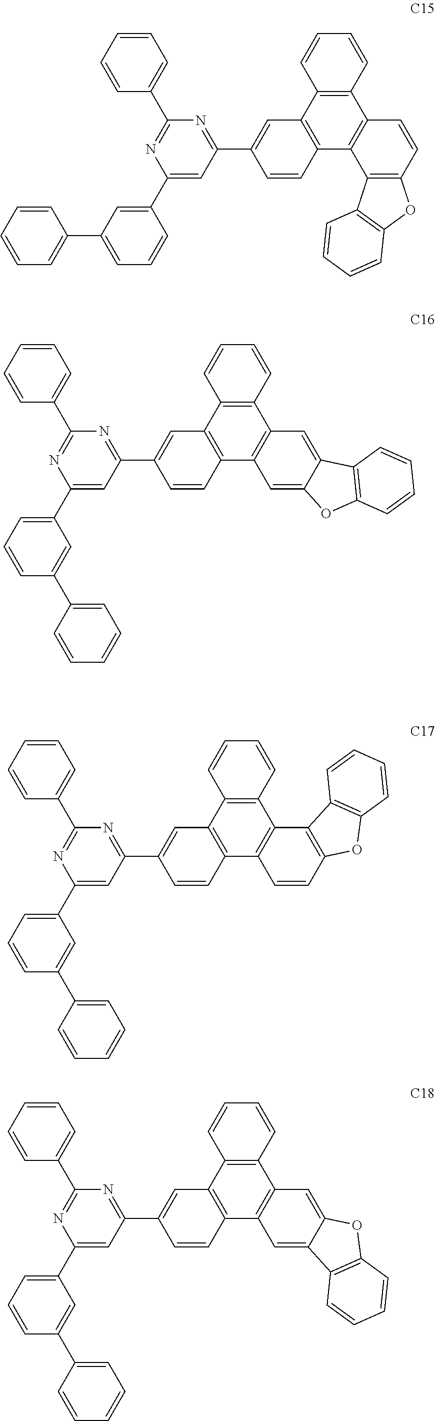 [SMILES: c1ccc(-c2cccc(-c3cc(-c4ccc5c(c4)c4ccccc4c4ccc6oc7ccccc7c6c45)nc(-c4ccccc4)n3)c2)cc1 ; c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4cc6oc7ccccc7c6cc54)nc(-c4ccccc4)n3)c2)cc1]

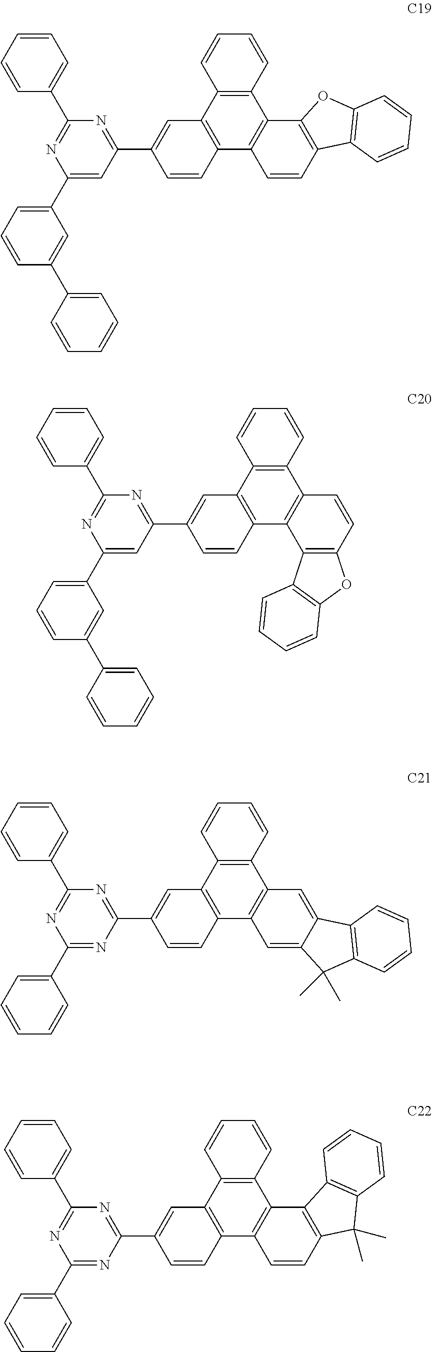 [SMILES: c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1 ; CC1(C)c2ccccc2-c2cc3c4ccccc4c4cc(-c5nc(-c6ccccc6)nc(-c6ccccc6)n5)ccc4c3cc21 ; CC1(C)c2ccccc2-c2c1ccc1c3ccc(-c4nc(-c5ccccc5)nc(-c5ccccc5)n4)cc3c3ccccc3c21]

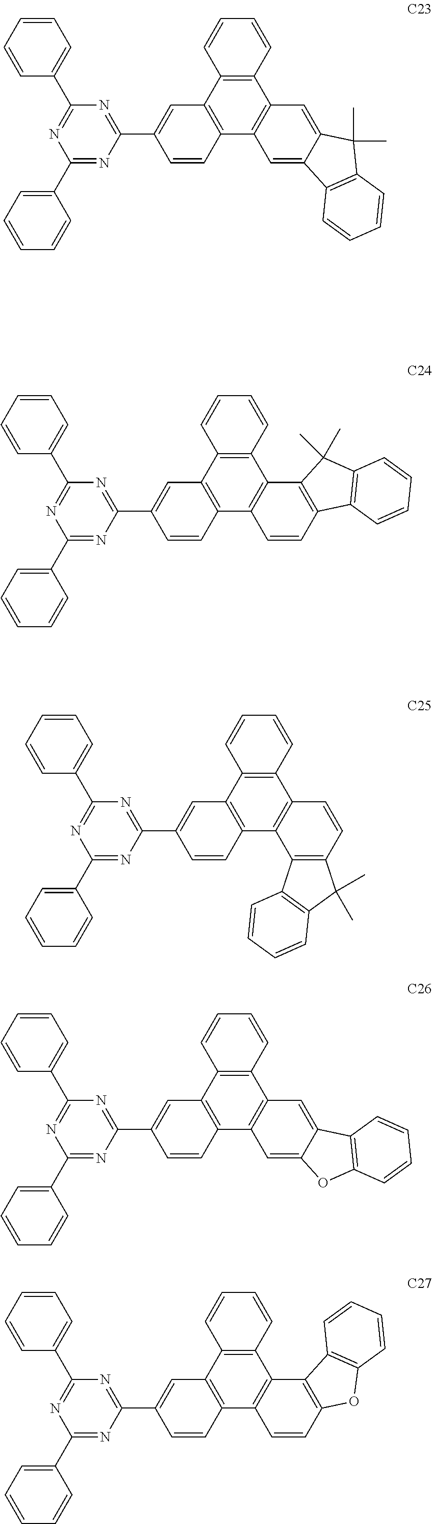 [SMILES: CC1(C)c2ccccc2-c2cc3c4ccc(-c5nc(-c6ccccc6)nc(-c6ccccc6)n5)cc4c4ccccc4c3cc21 ; CC1(C)c2ccccc2-c2ccc3c4ccc(-c5nc(-c6ccccc6)nc(-c6ccccc6)n5)cc4c4ccccc4c3c21 ; CC1(C)c2ccccc2-c2c1ccc1c3ccccc3c3cc(-c4nc(-c5ccccc5)nc(-c5ccccc5)n4)ccc3c21 ; c1ccc(-c2nc(-c3ccccc3)nc(-c3ccc4c(c3)c3ccccc3c3cc5c(cc43)oc3ccccc35)n2)cc1 ; c1ccc(-c2nc(-c3ccccc3)nc(-c3ccc4c(c3)c3ccccc3c3c4ccc4oc5ccccc5c43)n2)cc1]

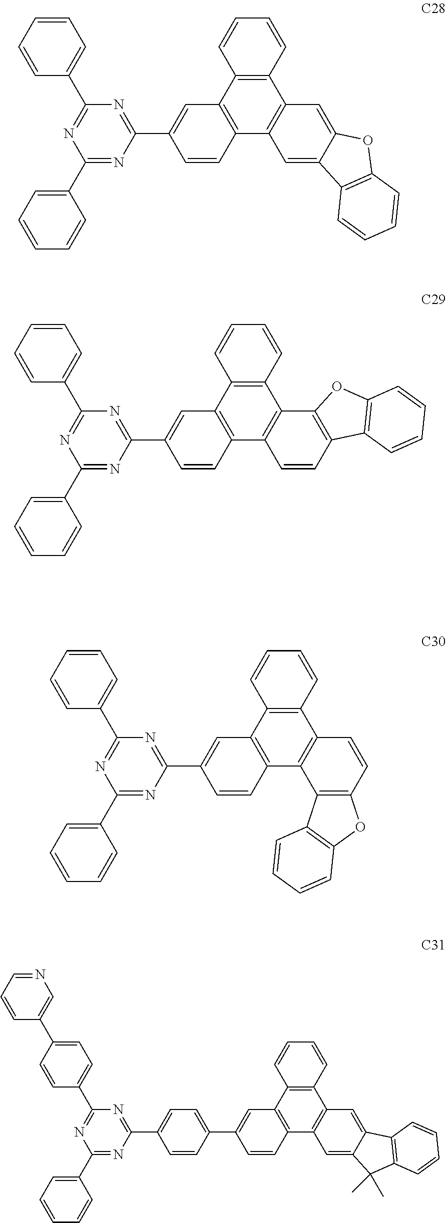 [SMILES: c1ccc(-c2nc(-c3ccccc3)nc(-c3ccc4c(c3)c3ccccc3c3cc5oc6ccccc6c5cc43)n2)cc1 ; c1ccc(-c2nc(-c3ccccc3)nc(-c3ccc4c(c3)c3ccccc3c3c4ccc4c5ccccc5oc43)n2)cc1 ; c1ccc(-c2nc(-c3ccccc3)nc(-c3ccc4c(c3)c3ccccc3c3ccc5oc6ccccc6c5c34)n2)cc1 ; CC1(C)c2ccccc2-c2cc3c4ccccc4c4cc(-c5ccc(-c6nc(-c7ccccc7)nc(-c7ccc(-c8cccnc8)cc7)n6)cc5)ccc4c3cc21]

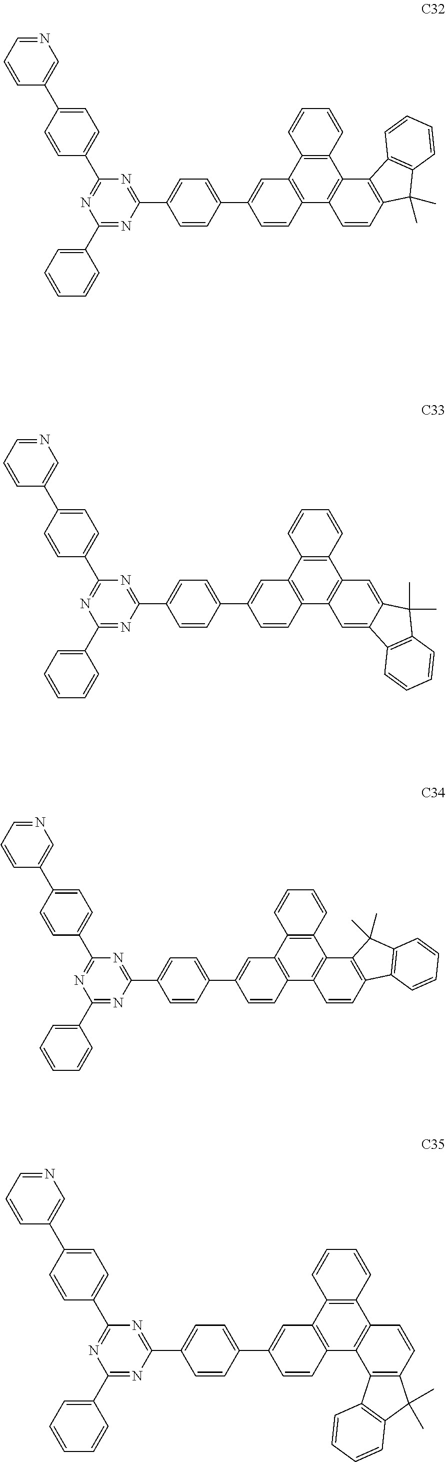 [SMILES: CC1(C)c2ccccc2-c2c1ccc1c3ccc(-c4ccc(-c5nc(-c6ccccc6)nc(-c6ccc(-c7cccnc7)cc6)n5)cc4)cc3c3ccccc3c21 ; CC1(C)c2ccccc2-c2cc3c4ccc(-c5ccc(-c6nc(-c7ccccc7)nc(-c7ccc(-c8cccnc8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8cccnc8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7cccnc7)cc6)n5)cc4)ccc3c21]

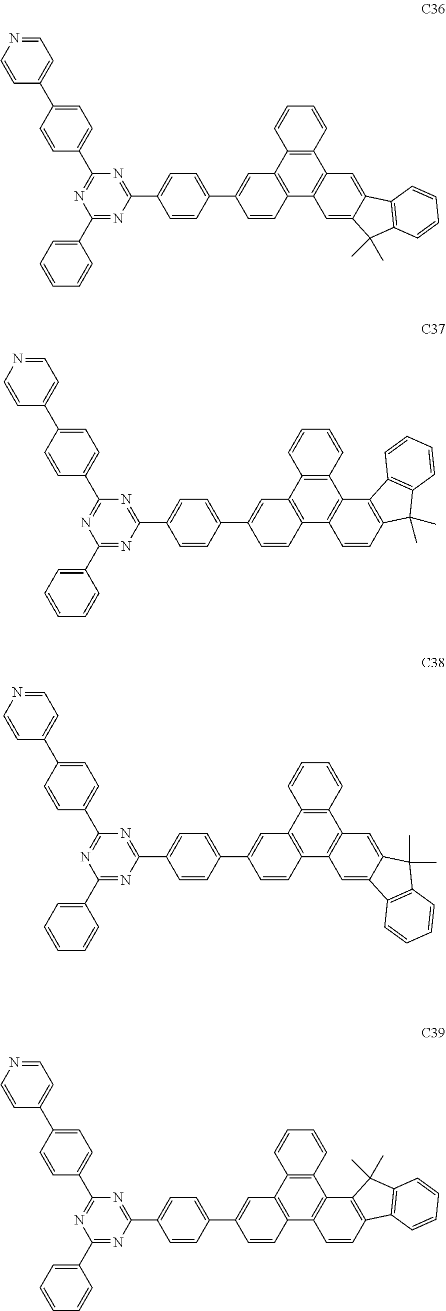 [SMILES: CC1(C)c2ccccc2-c2cc3c4ccccc4c4cc(-c5ccc(-c6nc(-c7ccccc7)nc(-c7ccc(-c8ccncc8)cc7)n6)cc5)ccc4c3cc21 ; CC1(C)c2ccccc2-c2c1ccc1c3ccc(-c4ccc(-c5nc(-c6ccccc6)nc(-c6ccc(-c7ccncc7)cc6)n5)cc4)cc3c3ccccc3c21 ; CC1(C)c2ccccc2-c2cc3c4ccc(-c5ccc(-c6nc(-c7ccccc7)nc(-c7ccc(-c8ccncc8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8ccncc8)cc7)n6)cc5)cc4c4ccccc4c3c21]

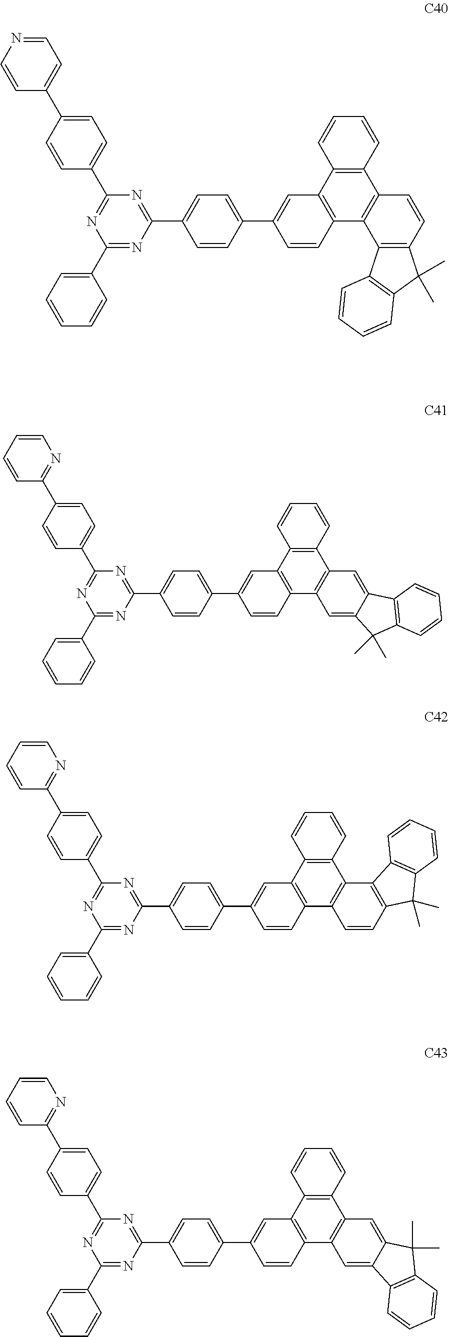 [SMILES: CC1(C)c2ccccc2-c2c1ccc1c3ccccc3c3cc(-c4ccc(-c5nc(-c6ccccc6)nc(-c6ccc(-c7ccncc7)cc6)n5)cc4)ccc3c21 ; CC1(C)c2ccccc2-c2cc3c4ccccc4c4cc(-c5ccc(-c6nc(-c7ccccc7)nc(-c7ccc(-c8ccccn8)cc7)n6)cc5)ccc4c3cc21 ; CC1(C)c2ccccc2-c2c1ccc1c3ccc(-c4ccc(-c5nc(-c6ccccc6)nc(-c6ccc(-c7ccccn7)cc6)n5)cc4)cc3c3ccccc3c21 ; CC1(C)c2ccccc2-c2cc3c4ccc(-c5ccc(-c6nc(-c7ccccc7)nc(-c7ccc(-c8ccccn8)cc7)n6)cc5)cc4c4ccccc4c3cc21]

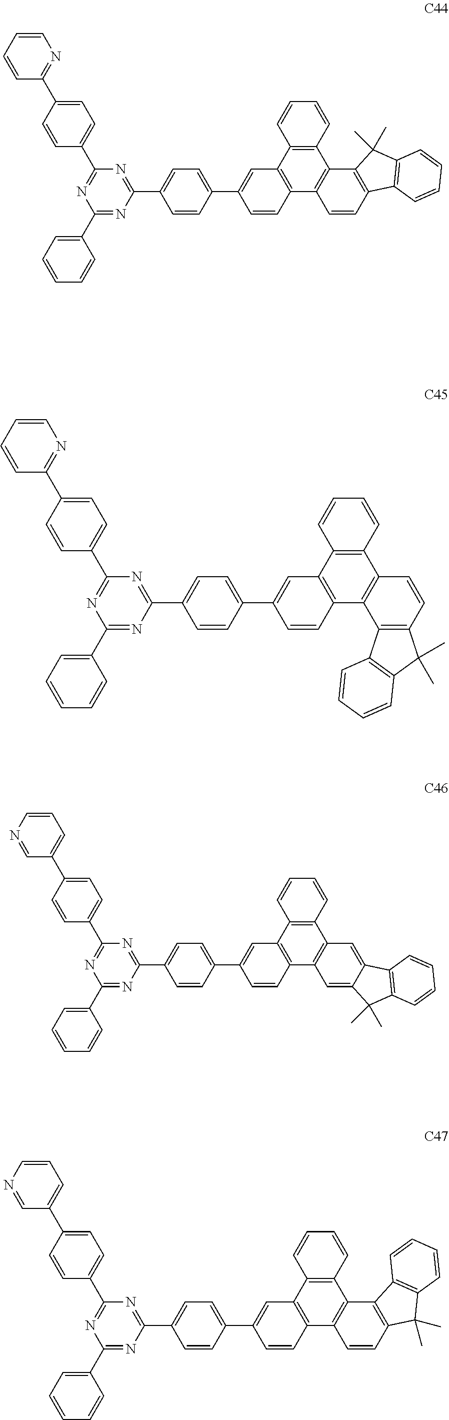 [SMILES: CC1(C)c2ccccc2-c2ccc3c4ccc(-c5ccc(-c6nc(-c7ccccc7)nc(-c7ccc(-c8ccccn8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7ccccn7)cc6)n5)cc4)ccc3c21 ; CC1(C)c2ccccc2-c2cc3c4ccccc4c4cc(-c5ccc(-c6nc(-c7ccccc7)nc(-c7ccc(-c8cccnc8)cc7)n6)cc5)ccc4c3cc21 ; CC1(C)c2ccccc2-c2c1ccc1c3ccc(-c4ccc(-c5nc(-c6ccccc6)nc(-c6ccc(-c7cccnc7)cc6)n5)cc4)cc3c3ccccc3c21]

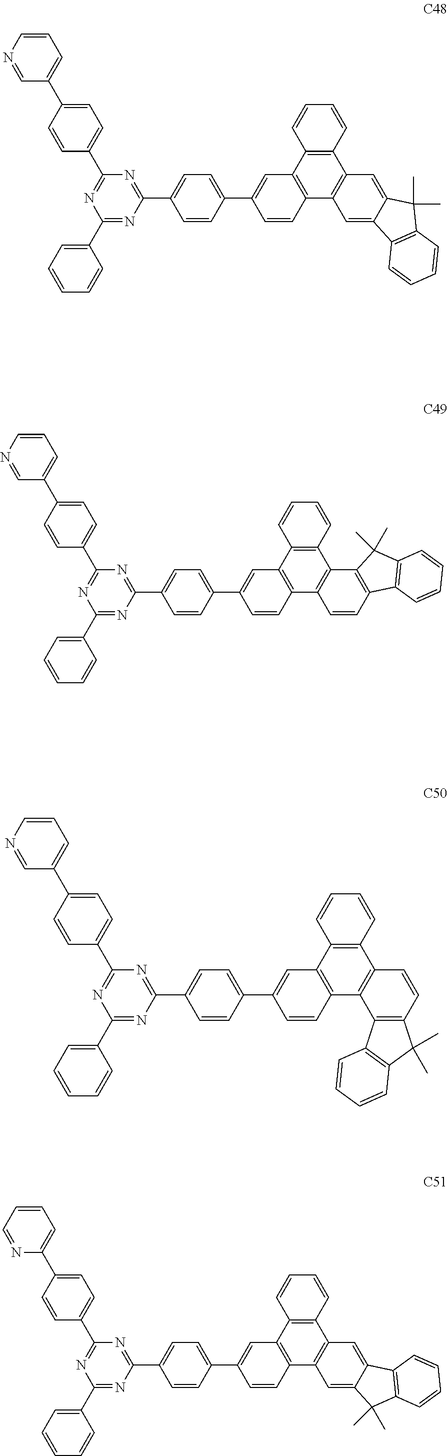 [SMILES: CC1(C)c2ccccc2-c2cc3c4ccc(-c5ccc(-c6nc(-c7ccccc7)nc(-c7ccc(-c8cccnc8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8cccnc8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7cccnc7)cc6)n5)cc4)ccc3c21 ; CC1(C)c2ccccc2-c2cc3c4ccccc4c4cc(-c5ccc(-c6nc(-c7ccccc7)nc(-c7ccc(-c8ccccn8)cc7)n6)cc5)ccc4c3cc21]

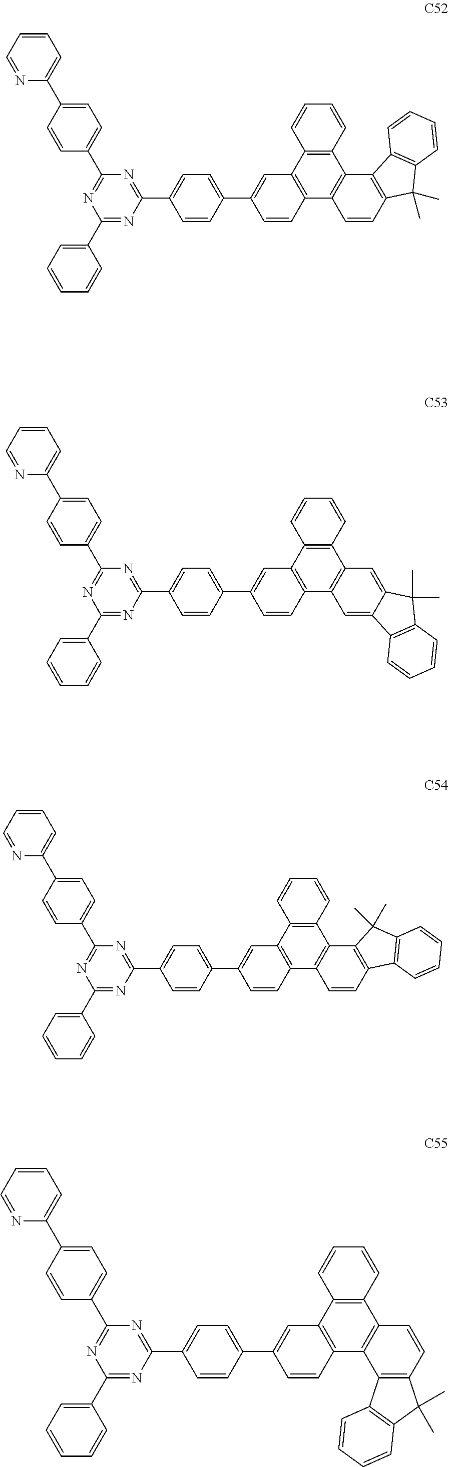 [SMILES: CC1(C)c2ccccc2-c2c1ccc1c3ccc(-c4ccc(-c5nc(-c6ccccc6)nc(-c6ccc(-c7ccccn7)cc6)n5)cc4)cc3c3ccccc3c21 ; CC1(C)c2ccccc2-c2cc3c4ccc(-c5ccc(-c6nc(-c7ccccc7)nc(-c7ccc(-c8ccccn8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8ccccn8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7ccccn7)cc6)n5)cc4)ccc3c21]

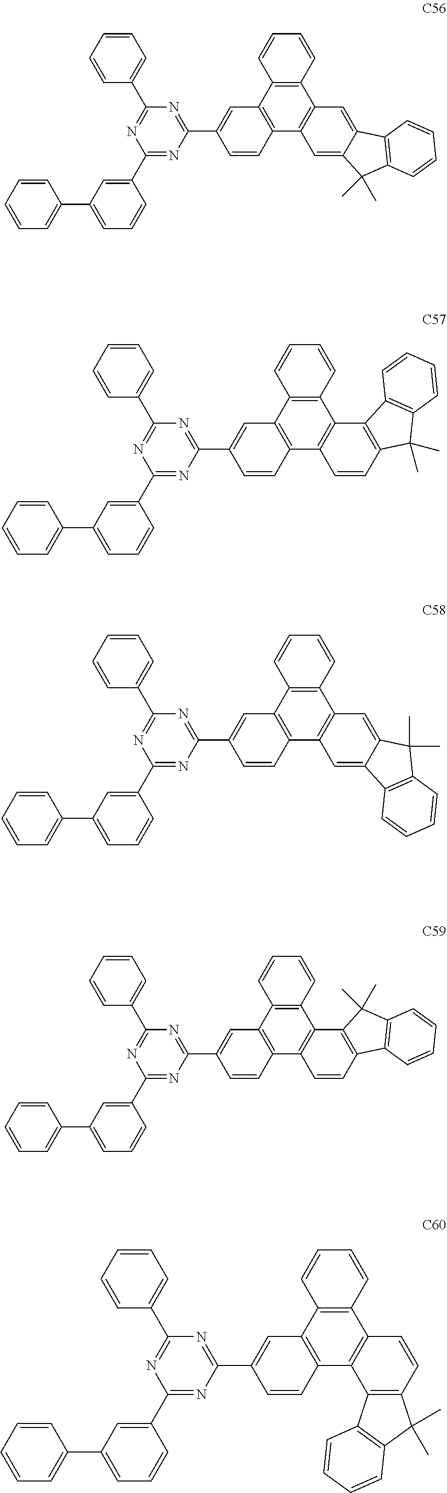 [SMILES: CC1(C)c2ccccc2-c2cc3c4ccccc4c4cc(-c5nc(-c6ccccc6)nc(-c6cccc(-c7ccccc7)c6)n5)ccc4c3cc21 ; CC1(C)c2ccccc2-c2c1ccc1c3ccc(-c4nc(-c5ccccc5)nc(-c5cccc(-c6ccccc6)c5)n4)cc3c3ccccc3c21 ; CC1(C)c2ccccc2-c2cc3c4ccc(-c5nc(-c6ccccc6)nc(-c6cccc(-c7ccccc7)c6)n5)cc4c4ccccc4c3cc21 ; CC1(C)c2ccccc2-c2ccc3c4ccc(-c5nc(-c6ccccc6)nc(-c6cccc(-c7ccccc7)c6)n5)cc4c4ccccc4c3c21 ; CC1(C)c2ccccc2-c2c1ccc1c3ccccc3c3cc(-c4nc(-c5ccccc5)nc(-c5cccc(-c6ccccc6)c5)n4)ccc3c21]

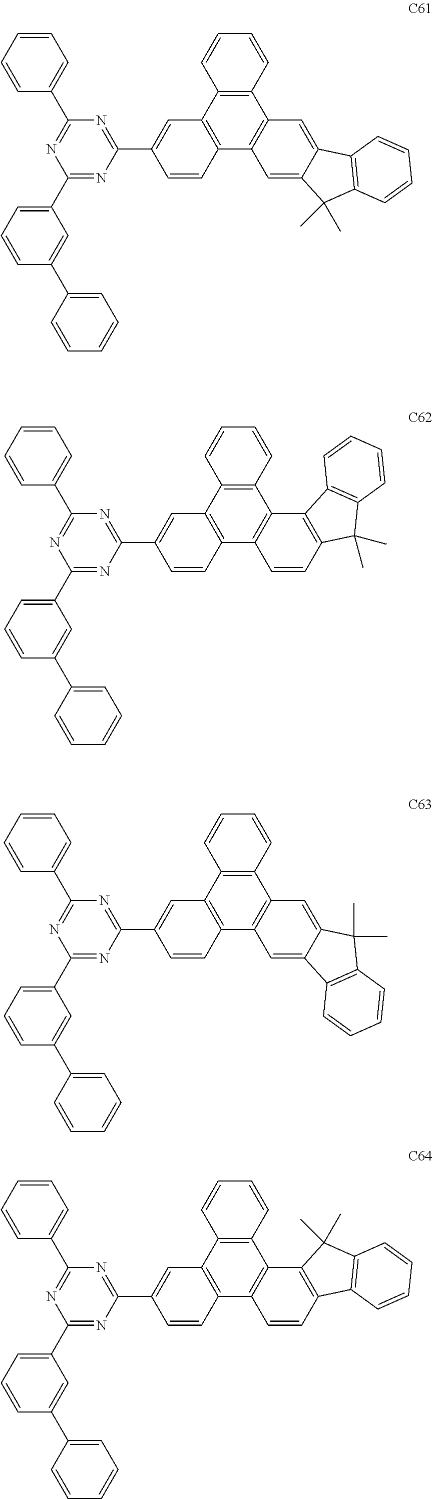 [SMILES: CC1(C)c2ccccc2-c2cc3c4ccccc4c4cc(-c5nc(-c6ccccc6)nc(-c6cccc(-c7ccccc7)c6)n5)ccc4c3cc21 ; CC1(C)c2ccccc2-c2c1ccc1c3ccc(-c4nc(-c5ccccc5)nc(-c5cccc(-c6ccccc6)c5)n4)cc3c3ccccc3c21 ; CC1(C)c2ccccc2-c2cc3c4ccc(-c5nc(-c6ccccc6)nc(-c6cccc(-c7ccccc7)c6)n5)cc4c4ccccc4c3cc21 ; CC1(C)c2ccccc2-c2ccc3c4ccc(-c5nc(-c6ccccc6)nc(-c6cccc(-c7ccccc7)c6)n5)cc4c4ccccc4c3c21]

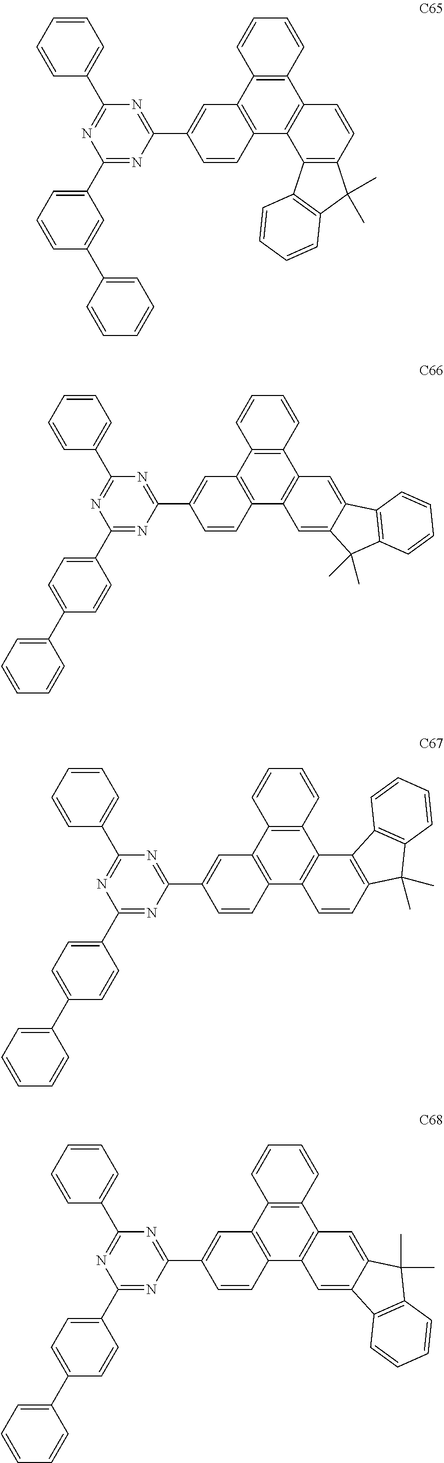 [SMILES: CC1(C)c2ccccc2-c2c1ccc1c3ccccc3c3cc(-c4nc(-c5ccccc5)nc(-c5cccc(-c6ccccc6)c5)n4)ccc3c21 ; CC1(C)c2ccccc2-c2cc3c4ccccc4c4cc(-c5nc(-c6ccccc6)nc(-c6ccc(-c7ccccc7)cc6)n5)ccc4c3cc21 ; CC1(C)c2ccccc2-c2c1ccc1c3ccc(-c4nc(-c5ccccc5)nc(-c5ccc(-c6ccccc6)cc5)n4)cc3c3ccccc3c21 ; CC1(C)c2ccccc2-c2cc3c4ccc(-c5nc(-c6ccccc6)nc(-c6ccc(-c7ccccc7)cc6)n5)cc4c4ccccc4c3cc21]

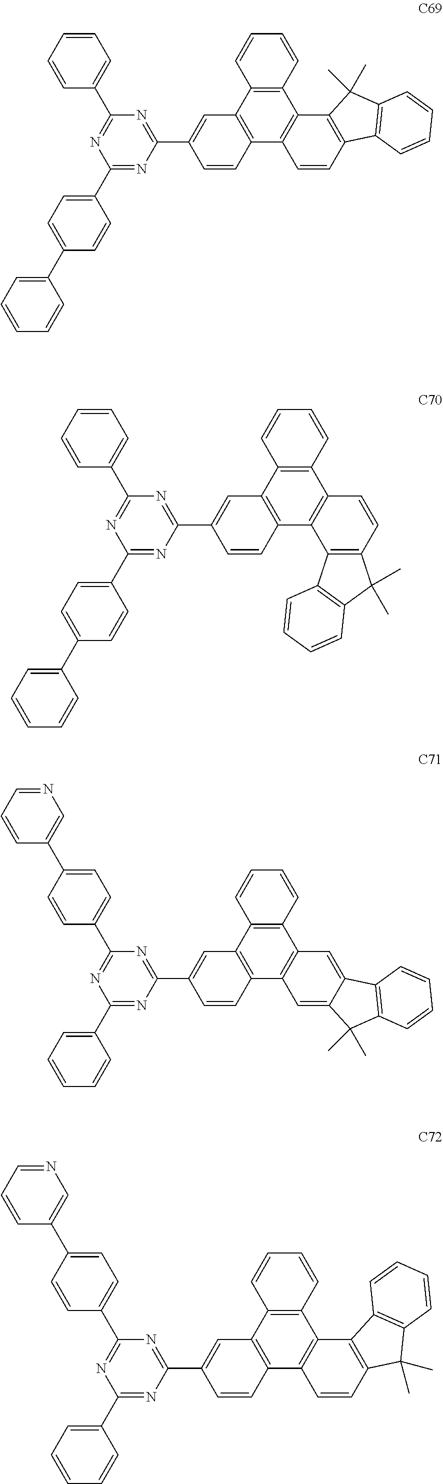 [SMILES: CC1(C)c2ccccc2-c2ccc3c4ccc(-c5nc(-c6ccccc6)nc(-c6ccc(-c7ccccc7)cc6)n5)cc4c4ccccc4c3c21 ; CC1(C)c2ccccc2-c2c1ccc1c3ccccc3c3cc(-c4nc(-c5ccccc5)nc(-c5ccc(-c6ccccc6)cc5)n4)ccc3c21 ; CC1(C)c2ccccc2-c2cc3c4ccccc4c4cc(-c5nc(-c6ccccc6)nc(-c6ccc(-c7cccnc7)cc6)n5)ccc4c3cc21 ; CC1(C)c2ccccc2-c2c1ccc1c3ccc(-c4nc(-c5ccccc5)nc(-c5ccc(-c6cccnc6)cc5)n4)cc3c3ccccc3c21]

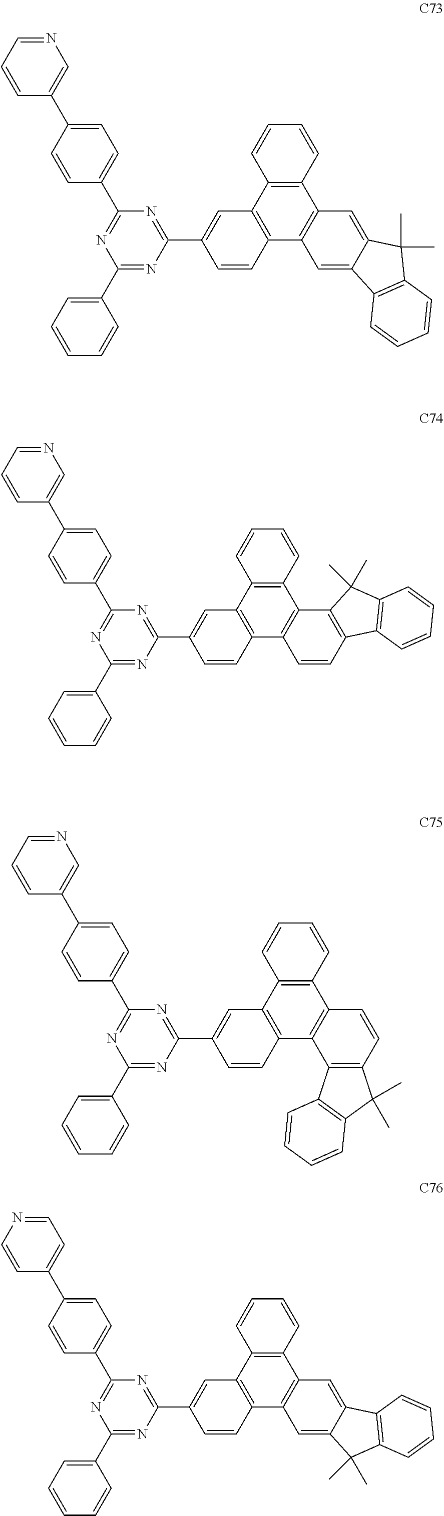 [SMILES: CC1(C)c2ccccc2-c2cc3c4ccc(-c5nc(-c6ccccc6)nc(-c6ccc(-c7cccnc7)cc6)n5)cc4c4ccccc4c3cc21 ; CC1(C)c2ccccc2-c2ccc3c4ccc(-c5nc(-c6ccccc6)nc(-c6ccc(-c7cccnc7)cc6)n5)cc4c4ccccc4c3c21 ; CC1(C)c2ccccc2-c2c1ccc1c3ccccc3c3cc(-c4nc(-c5ccccc5)nc(-c5ccc(-c6cccnc6)cc5)n4)ccc3c21 ; CC1(C)c2ccccc2-c2cc3c4ccccc4c4cc(-c5nc(-c6ccccc6)nc(-c6ccc(-c7ccncc7)cc6)n5)ccc4c3cc21]

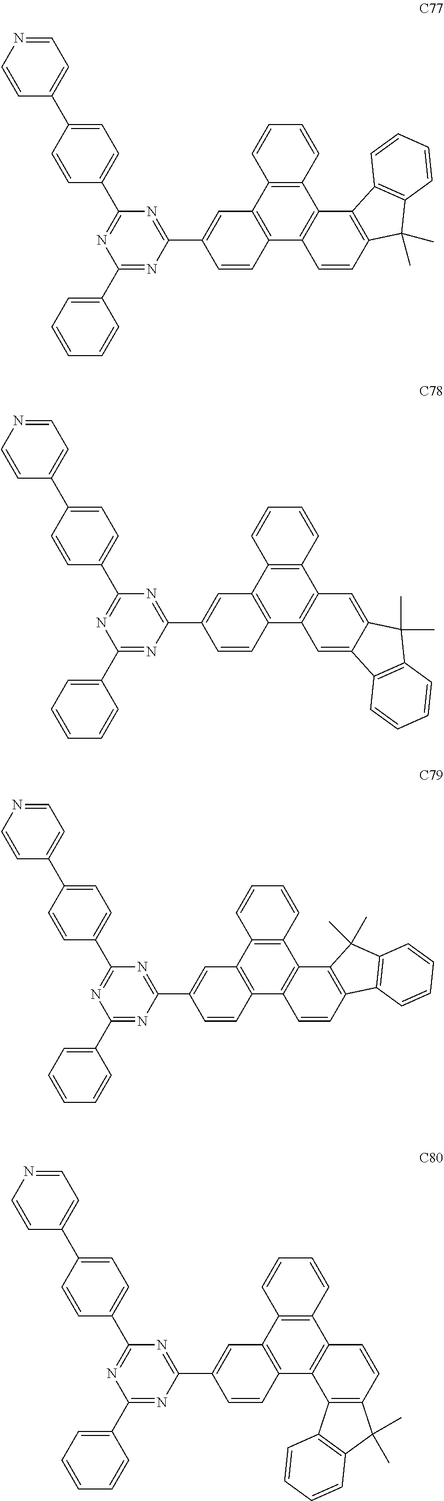 [SMILES: CC1(C)c2ccccc2-c2c1ccc1c3ccc(-c4nc(-c5ccccc5)nc(-c5ccc(-c6ccncc6)cc5)n4)cc3c3ccccc3c21 ; CC1(C)c2ccccc2-c2cc3c4ccc(-c5nc(-c6ccccc6)nc(-c6ccc(-c7ccncc7)cc6)n5)cc4c4ccccc4c3cc21 ; CC1(C)c2ccccc2-c2ccc3c4ccc(-c5nc(-c6ccccc6)nc(-c6ccc(-c7ccncc7)cc6)n5)cc4c4ccccc4c3c21 ; CC1(C)c2ccccc2-c2c1ccc1c3ccccc3c3cc(-c4nc(-c5ccccc5)nc(-c5ccc(-c6ccncc6)cc5)n4)ccc3c21]

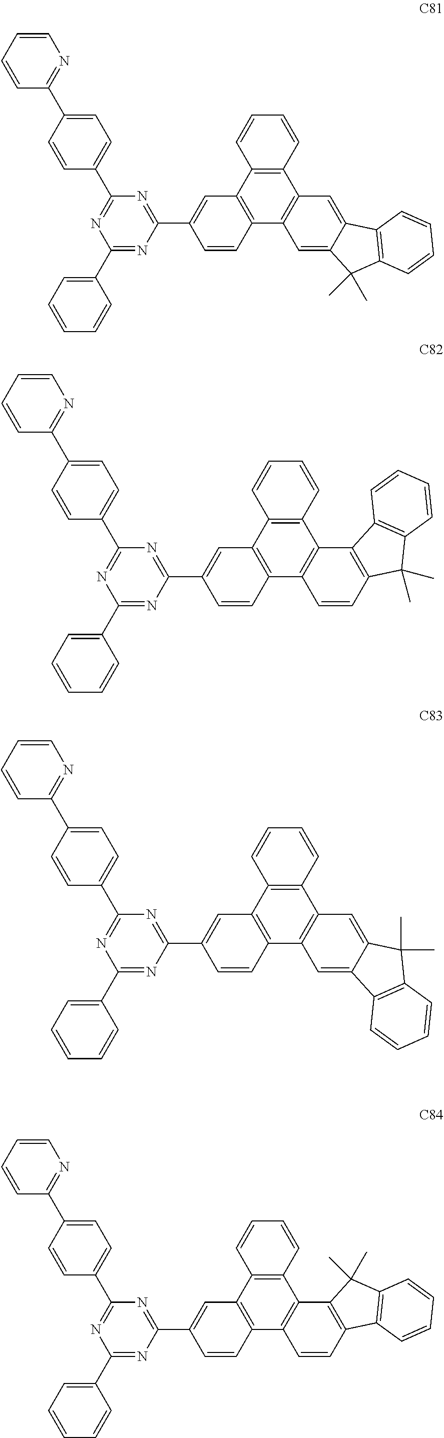

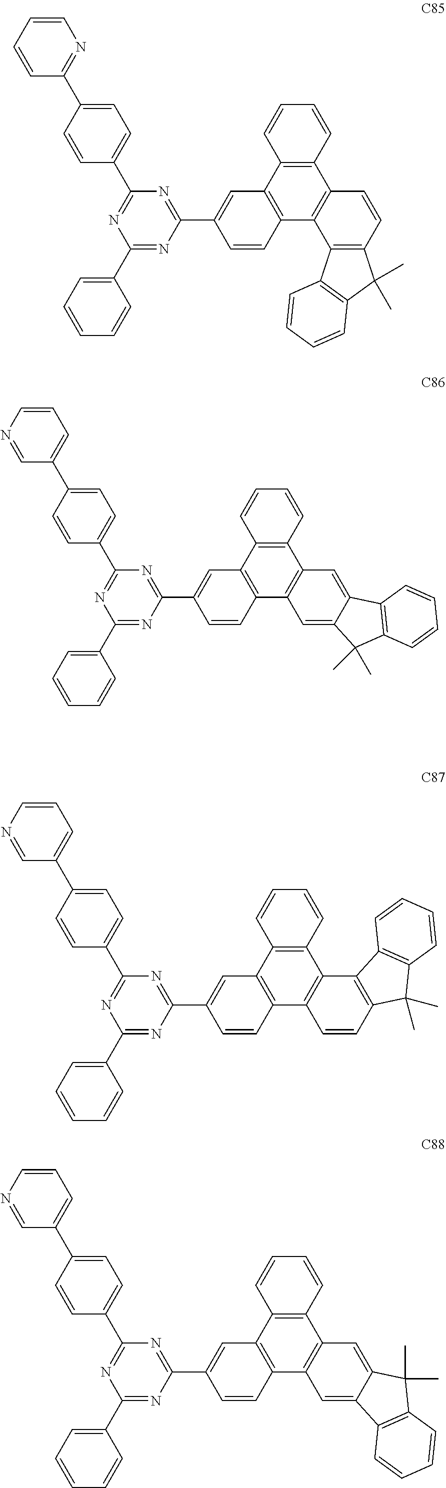 [SMILES: CC1(C)c2ccccc2-c2c1ccc1c3ccccc3c3cc(-c4nc(-c5ccccc5)nc(-c5ccc(-c6ccccn6)cc5)n4)ccc3c21 ; CC1(C)c2ccccc2-c2cc3c4ccccc4c4cc(-c5nc(-c6ccccc6)nc(-c6ccc(-c7cccnc7)cc6)n5)ccc4c3cc21 ; CC1(C)c2ccccc2-c2c1ccc1c3ccc(-c4nc(-c5ccccc5)nc(-c5ccc(-c6cccnc6)cc5)n4)cc3c3ccccc3c21 ; CC1(C)c2ccccc2-c2cc3c4ccc(-c5nc(-c6ccccc6)nc(-c6ccc(-c7cccnc7)cc6)n5)cc4c4ccccc4c3cc21]

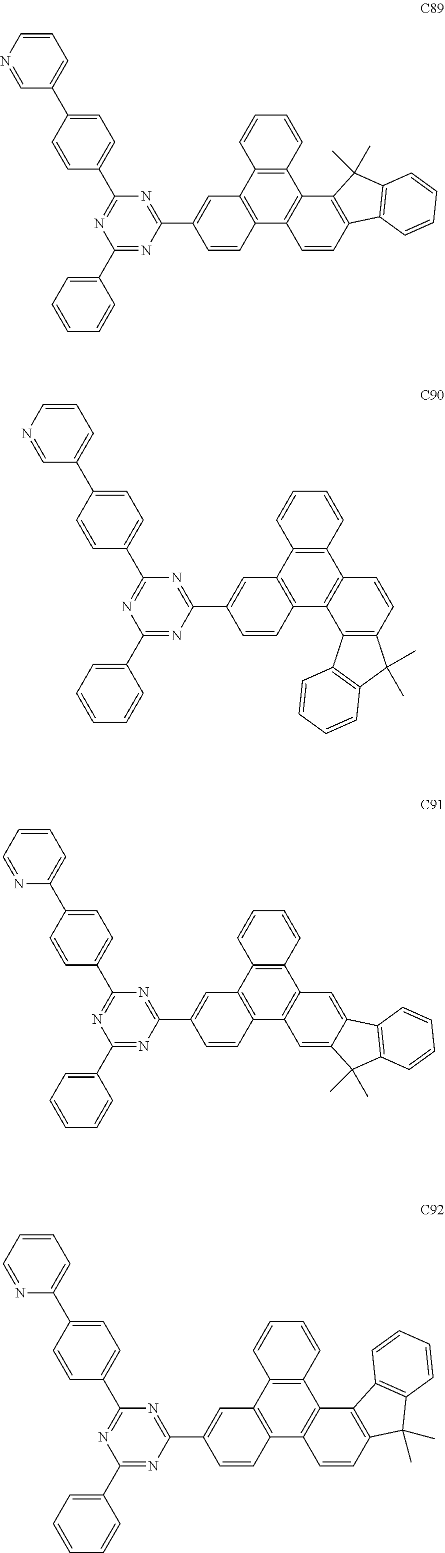 [SMILES: CC1(C)c2ccccc2-c2ccc3c4ccc(-c5nc(-c6ccccc6)nc(-c6ccc(-c7cccnc7)cc6)n5)cc4c4ccccc4c3c21 ; CC1(C)c2ccccc2-c2c1ccc1c3ccccc3c3cc(-c4nc(-c5ccccc5)nc(-c5ccc(-c6cccnc6)cc5)n4)ccc3c21 ; CC1(C)c2ccccc2-c2cc3c4ccccc4c4cc(-c5nc(-c6ccccc6)nc(-c6ccc(-c7ccccn7)cc6)n5)ccc4c3cc21 ; CC1(C)c2ccccc2-c2c1ccc1c3ccc(-c4nc(-c5ccccc5)nc(-c5ccc(-c6ccccn6)cc5)n4)cc3c3ccccc3c21]

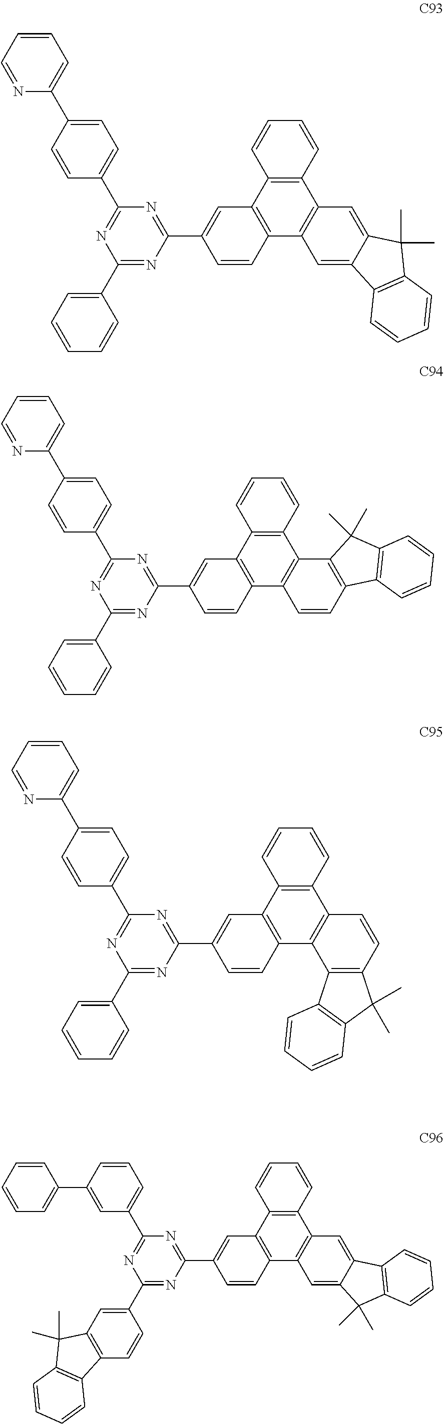 [SMILES: CC1(C)c2ccccc2-c2cc3c4ccc(-c5nc(-c6ccccc6)nc(-c6ccc(-c7ccccn7)cc6)n5)cc4c4ccccc4c3cc21 ; CC1(C)c2ccccc2-c2ccc3c4ccc(-c5nc(-c6ccccc6)nc(-c6ccc(-c7ccccn7)cc6)n5)cc4c4ccccc4c3c21 ; CC1(C)c2ccccc2-c2c1ccc1c3ccccc3c3cc(-c4nc(-c5ccccc5)nc(-c5ccc(-c6ccccn6)cc5)n4)ccc3c21 ; CC1(C)c2ccccc2-c2ccc(-c3nc(-c4cccc(-c5ccccc5)c4)nc(-c4ccc5c(c4)c4ccccc4c4cc6c(cc54)C(C)(C)c4ccccc4-6)n3)cc21]

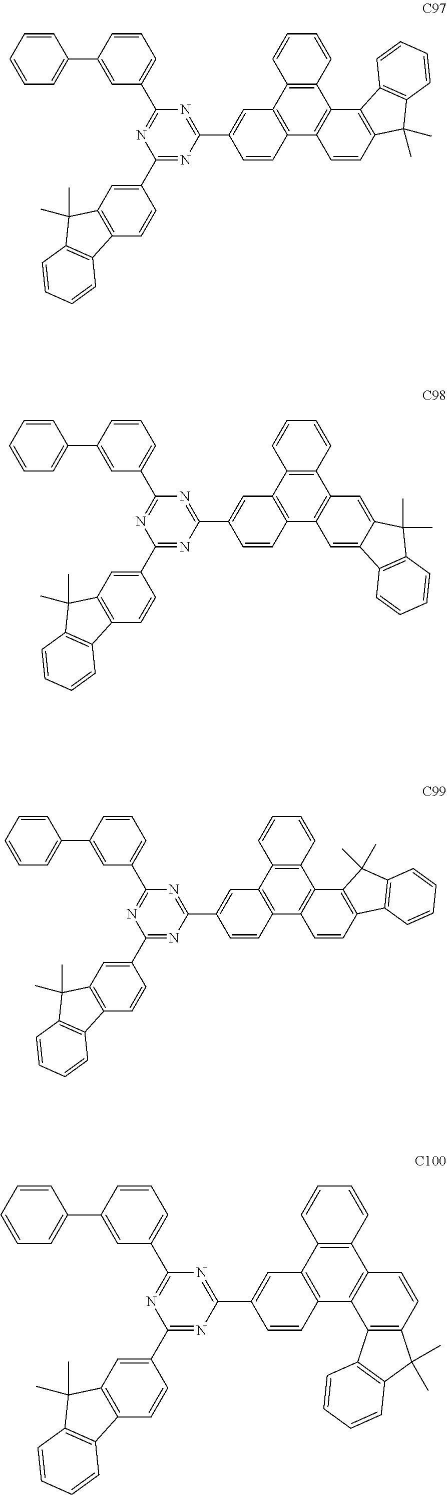 [SMILES: CC1(C)c2ccccc2-c2ccc(-c3nc(-c4cccc(-c5ccccc5)c4)nc(-c4ccc5c(c4)c4ccccc4c4c6c(ccc54)C(C)(C)c4ccccc4-6)n3)cc21 ; CC1(C)c2ccccc2-c2ccc(-c3nc(-c4cccc(-c5ccccc5)c4)nc(-c4ccc5c(c4)c4ccccc4c4cc6c(cc54)-c4ccccc4C6(C)C)n3)cc21 ; CC1(C)c2ccccc2-c2ccc(-c3nc(-c4cccc(-c5ccccc5)c4)nc(-c4ccc5c(c4)c4ccccc4c4c6c(ccc54)-c4ccccc4C6(C)C)n3)cc21 ; CC1(C)c2ccccc2-c2ccc(-c3nc(-c4cccc(-c5ccccc5)c4)nc(-c4ccc5c(c4)c4ccccc4c4ccc6c(c45)-c4ccccc4C6(C)C)n3)cc21]

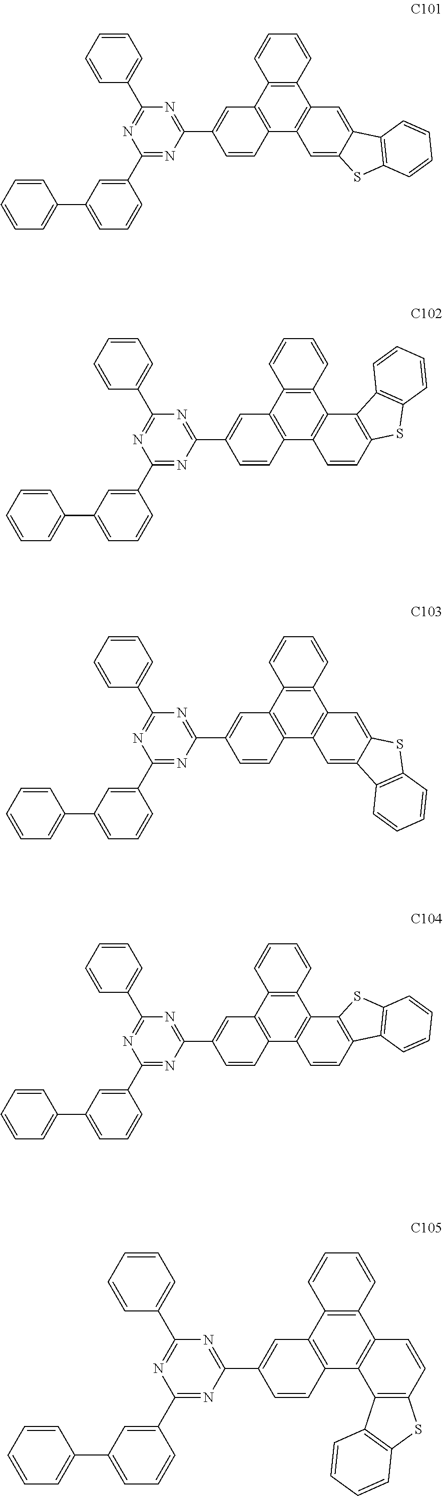 [SMILES: c1ccc(-c2cccc(-c3nc(-c4ccccc4)nc(-c4ccc5c(c4)c4ccccc4c4cc6c(cc54)sc4ccccc46)n3)c2)cc1 ; c1ccc(-c2cccc(-c3nc(-c4ccccc4)nc(-c4ccc5c(c4)c4ccccc4c4c5ccc5sc6ccccc6c54)n3)c2)cc1 ; c1ccc(-c2cccc(-c3nc(-c4ccccc4)nc(-c4ccc5c(c4)c4ccccc4c4cc6sc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6sc54)n3)c2)cc1 ; c1ccc(-c2cccc(-c3nc(-c4ccccc4)nc(-c4ccc5c(c4)c4ccccc4c4ccc6sc7ccccc7c6c45)n3)c2)cc1]

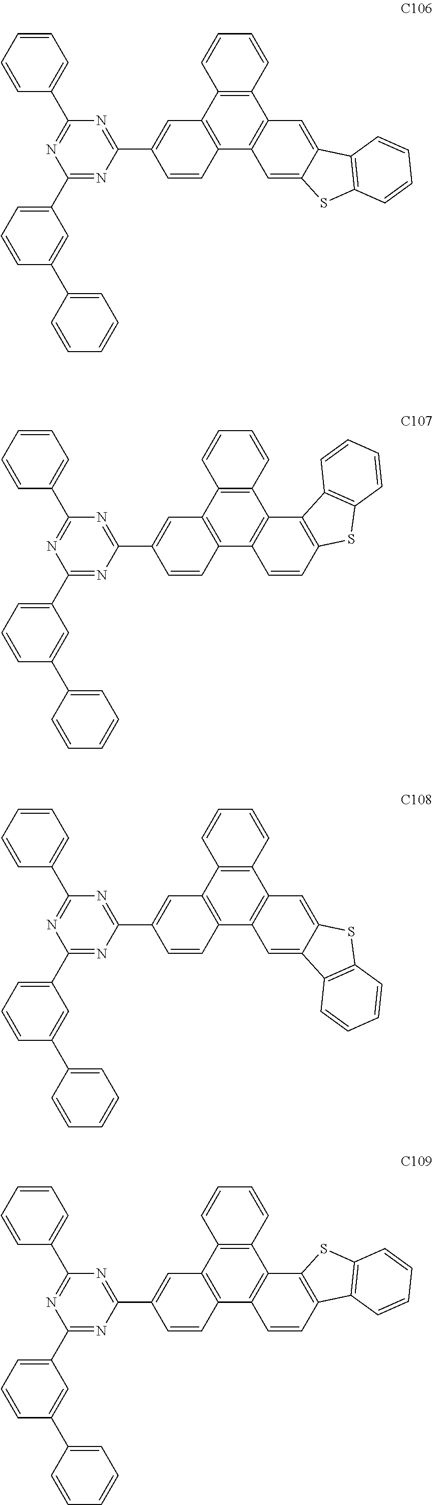 [SMILES: c1ccc(-c2cccc(-c3nc(-c4ccccc4)nc(-c4ccc5c(c4)c4ccccc4c4cc6c(cc54)sc4ccccc46)n3)c2)cc1 ; c1ccc(-c2cccc(-c3nc(-c4ccccc4)nc(-c4ccc5c(c4)c4ccccc4c4c5ccc5sc6ccccc6c54)n3)c2)cc1 ; c1ccc(-c2cccc(-c3nc(-c4ccccc4)nc(-c4ccc5c(c4)c4ccccc4c4cc6sc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6sc54)n3)c2)cc1]

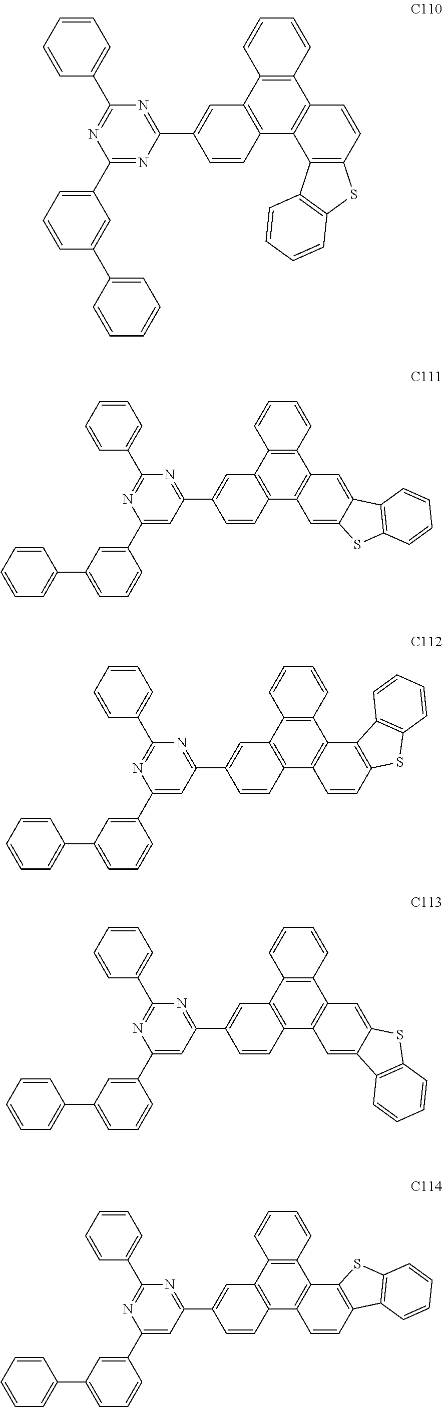 [SMILES: c1ccc(-c2cccc(-c3nc(-c4ccccc4)nc(-c4ccc5c(c4)c4ccccc4c4ccc6sc7ccccc7c6c45)n3)c2)cc1 ; c1ccc(-c2cccc(-c3cc(-c4ccc5c(c4)c4ccccc4c4cc6c(cc54)sc4ccccc46)nc(-c4ccccc4)n3)c2)cc1 ; c1ccc(-c2cccc(-c3cc(-c4ccc5c(c4)c4ccccc4c4c5ccc5sc6ccccc6c54)nc(-c4ccccc4)n3)c2)cc1 ; c1ccc(-c2cccc(-c3cc(-c4ccc5c(c4)c4ccccc4c4cc6sc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6sc54)nc(-c4ccccc4)n3)c2)cc1]

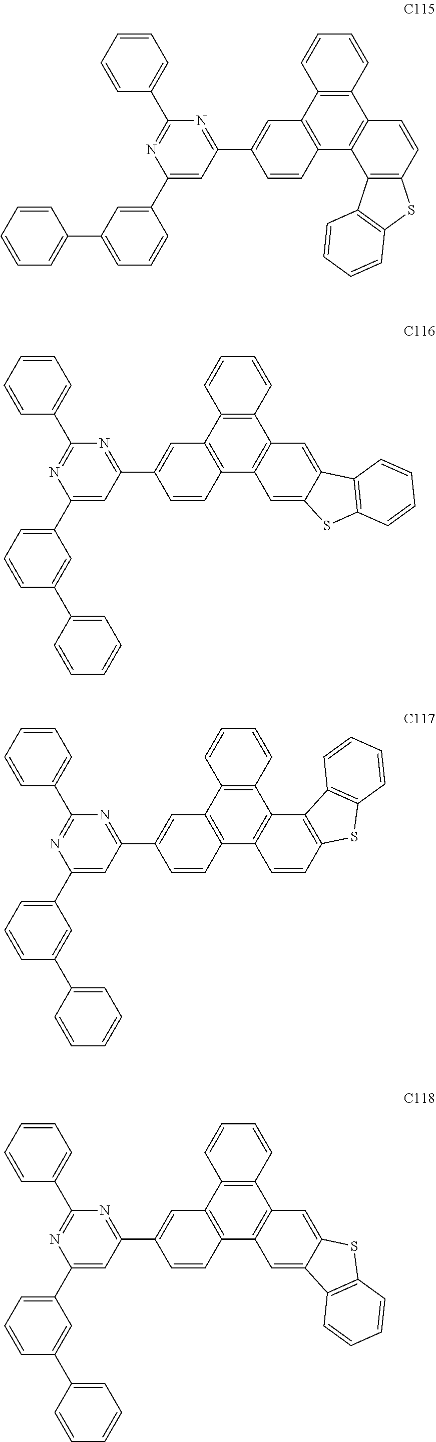 [SMILES: c1ccc(-c2cccc(-c3cc(-c4ccc5c(c4)c4ccccc4c4ccc6sc7ccccc7c6c45)nc(-c4ccccc4)n3)c2)cc1 ; c1ccc(-c2cccc(-c3cc(-c4ccc5c(c4)c4ccccc4c4cc6c(cc54)sc4ccccc46)nc(-c4ccccc4)n3)c2)cc1 ; c1ccc(-c2cccc(-c3cc(-c4ccc5c(c4)c4ccccc4c4c5ccc5sc6ccccc6c54)nc(-c4ccccc4)n3)c2)cc1 ; c1ccc(-c2cccc(-c3cc(-c4ccc5c(c4)c4ccccc4c4cc6sc7ccccc7c6cc54)nc(-c4ccccc4)n3)c2)cc1]

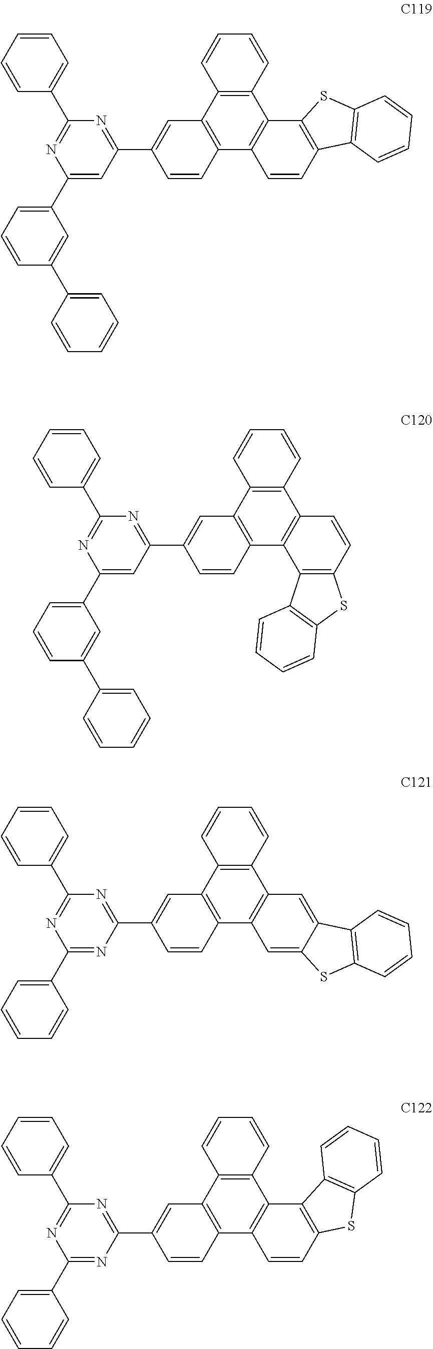 [SMILES: c1ccc(-c2cccc(-c3cc(-c4ccc5c(c4)c4ccccc4c4c5ccc5c6ccccc6sc54)nc(-c4ccccc4)n3)c2)cc1 ; c1ccc(-c2cccc(-c3cc(-c4ccc5c(c4)c4ccccc4c4ccc6sc7ccccc7c6c45)nc(-c4ccccc4)n3)c2)cc1 ; c1ccc(-c2nc(-c3ccccc3)nc(-c3ccc4c(c3)c3ccccc3c3cc5c(cc43)sc3ccccc35)n2)cc1 ; c1ccc(-c2nc(-c3ccccc3)nc(-c3ccc4c(c3)c3ccccc3c3c4ccc4sc5ccccc5c43)n2)cc1]

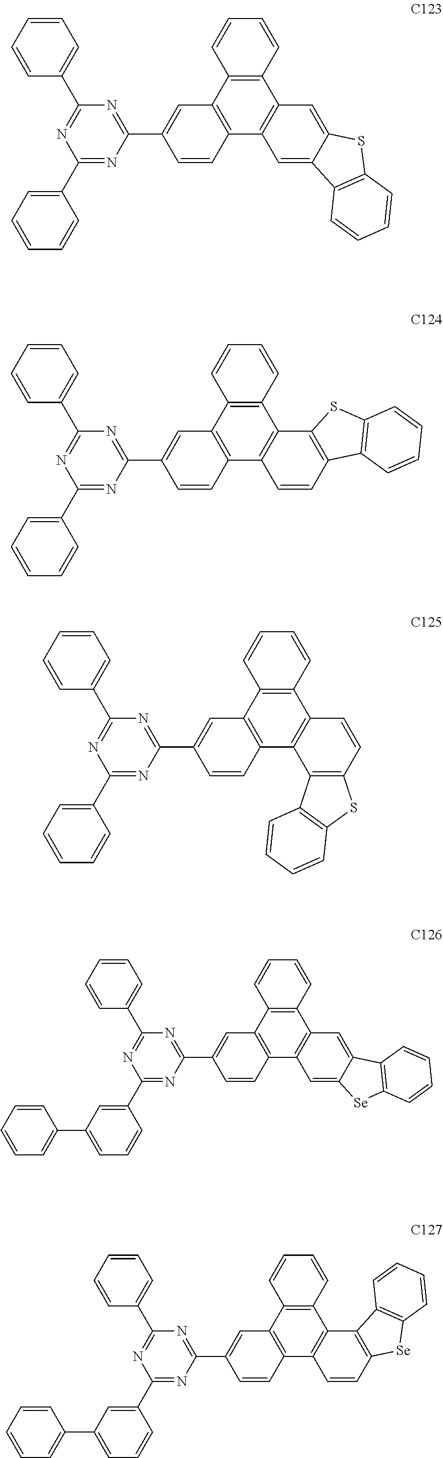 [SMILES: c1ccc(-c2nc(-c3ccccc3)nc(-c3ccc4c(c3)c3ccccc3c3cc5sc6ccccc6c5cc43)n2)cc1 ; c1ccc(-c2nc(-c3ccccc3)nc(-c3ccc4c(c3)c3ccccc3c3c4ccc4c5ccccc5sc43)n2)cc1 ; c1ccc(-c2nc(-c3ccccc3)nc(-c3ccc4c(c3)c3ccccc3c3ccc5sc6ccccc6c5c34)n2)cc1 ; c1ccc(-c2cccc(-c3nc(-c4ccccc4)nc(-c4ccc5c(c4)c4ccccc4c4cc6c(cc54)[se]c4ccccc46)n3)c2)cc1 ; c1ccc(-c2cccc(-c3nc(-c4ccccc4)nc(-c4ccc5c(c4)c4ccccc4c4c5ccc5[se]c6ccccc6c54)n3)c2)cc1]

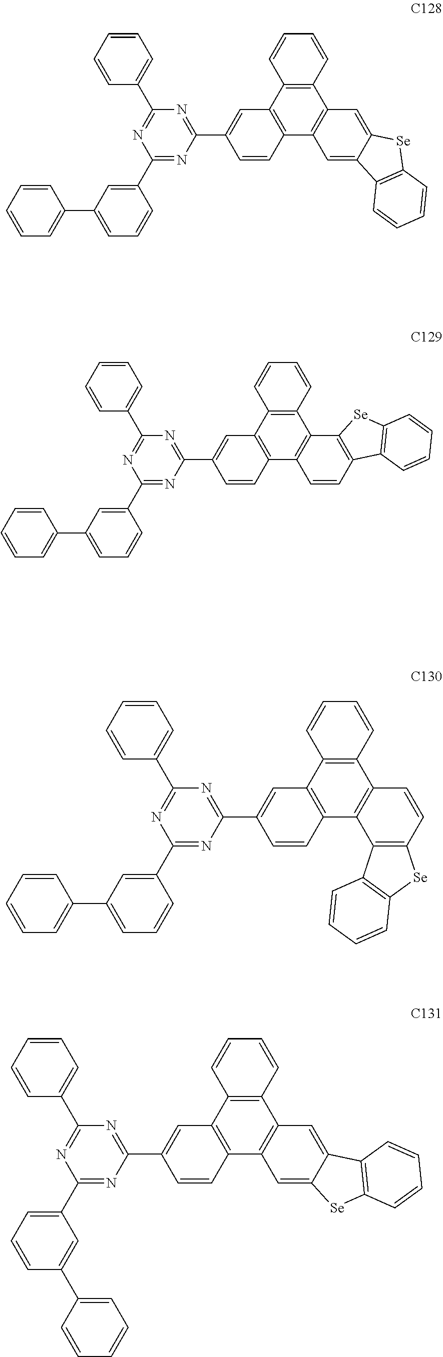 [SMILES: c1ccc(-c2cccc(-c3nc(-c4ccccc4)nc(-c4ccc5c(c4)c4ccccc4c4cc6[se]c7ccccc7c6cc54)n3)c2)cc1 ; c1ccc(-c2cccc(-c3nc(-c4ccccc4)nc(-c4ccc5c(c4)c4ccccc4c4c5ccc5c6ccccc6[se]c54)n3)c2)cc1 ; c1ccc(-c2cccc(-c3nc(-c4ccccc4)nc(-c4ccc5c(c4)c4ccccc4c4ccc6[se]c7ccccc7c6c45)n3)c2)cc1 ; c1ccc(-c2cccc(-c3nc(-c4ccccc4)nc(-c4ccc5c(c4)c4ccccc4c4cc6c(cc54)[se]c4ccccc46)n3)c2)cc1]

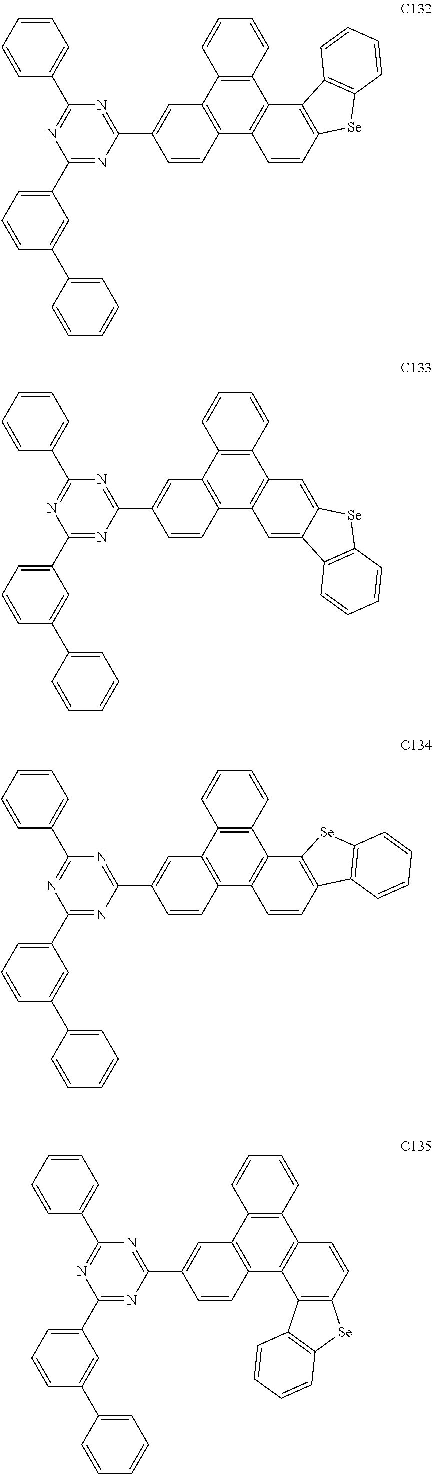 [SMILES: c1ccc(-c2cccc(-c3nc(-c4ccccc4)nc(-c4ccc5c(c4)c4ccccc4c4c5ccc5[se]c6ccccc6c54)n3)c2)cc1 ; c1ccc(-c2cccc(-c3nc(-c4ccccc4)nc(-c4ccc5c(c4)c4ccccc4c4cc6[se]c7ccccc7c6cc54)n3)c2)cc1 ; c1ccc(-c2cccc(-c3nc(-c4ccccc4)nc(-c4ccc5c(c4)c4ccccc4c4c5ccc5c6ccccc6[se]c54)n3)c2)cc1 ; c1ccc(-c2cccc(-c3nc(-c4ccccc4)nc(-c4ccc5c(c4)c4ccccc4c4ccc6[se]c7ccccc7c6c45)n3)c2)cc1]

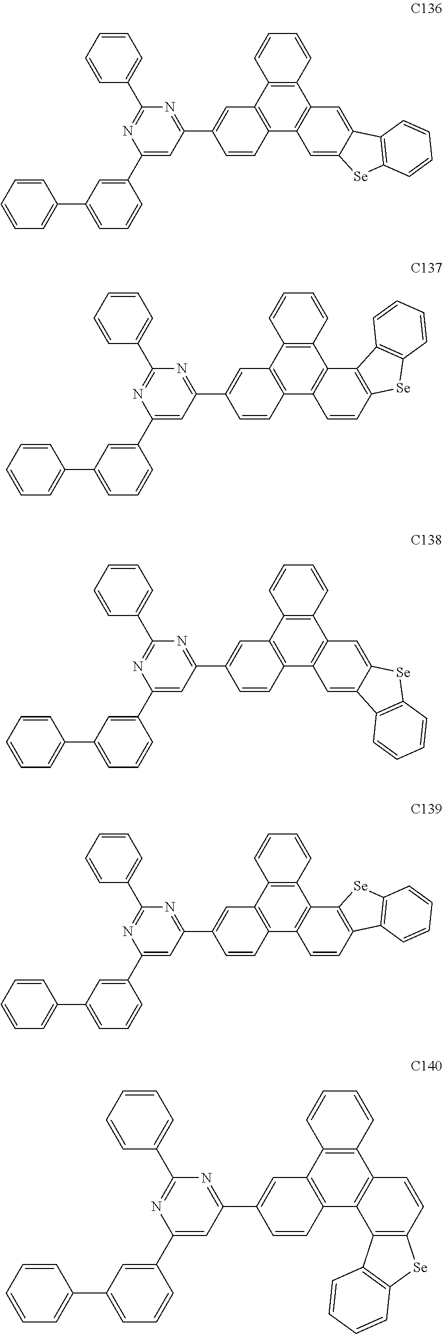 [SMILES: c1ccc(-c2cccc(-c3cc(-c4ccc5c(c4)c4ccccc4c4cc6c(cc54)[se]c4ccccc46)nc(-c4ccccc4)n3)c2)cc1 ; c1ccc(-c2cccc(-c3cc(-c4ccc5c(c4)c4ccccc4c4c5ccc5[se]c6ccccc6c54)nc(-c4ccccc4)n3)c2)cc1 ; c1ccc(-c2cccc(-c3cc(-c4ccc5c(c4)c4ccccc4c4cc6[se]c7ccccc7c6cc54)nc(-c4ccccc4)n3)c2)cc1 ; c1ccc(-c2cccc(-c3cc(-c4ccc5c(c4)c4ccccc4c4c5ccc5c6ccccc6[se]c54)nc(-c4ccccc4)n3)c2)cc1 ; c1ccc(-c2cccc(-c3cc(-c4ccc5c(c4)c4ccccc4c4ccc6[se]c7ccccc7c6c45)nc(-c4ccccc4)n3)c2)cc1]

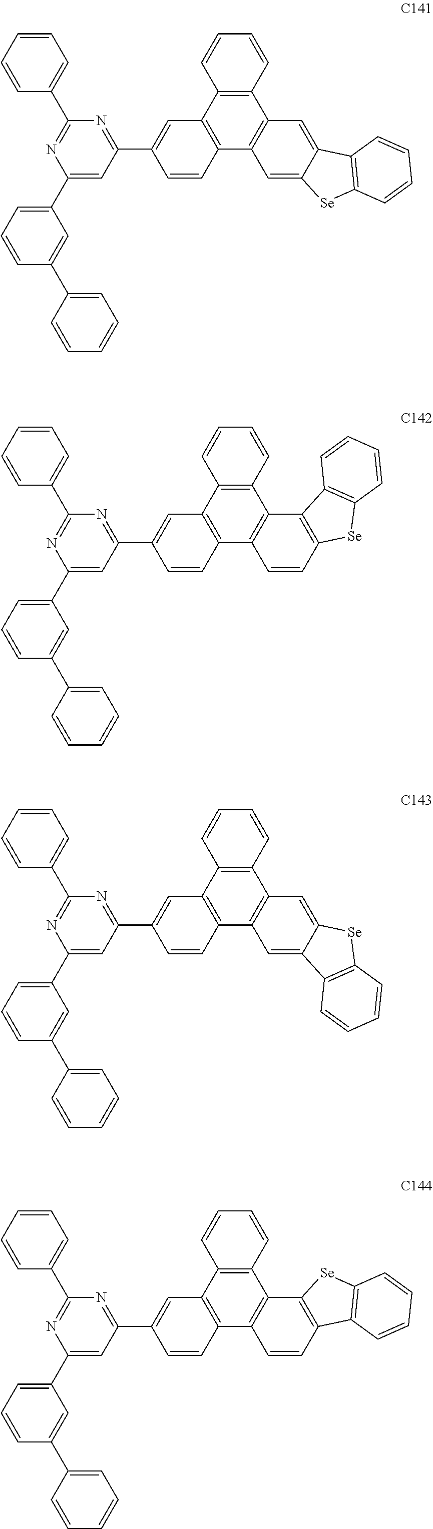 [SMILES: c1ccc(-c2cccc(-c3cc(-c4ccc5c(c4)c4ccccc4c4cc6c(cc54)[se]c4ccccc46)nc(-c4ccccc4)n3)c2)cc1 ; c1ccc(-c2cccc(-c3cc(-c4ccc5c(c4)c4ccccc4c4c5ccc5[se]c6ccccc6c54)nc(-c4ccccc4)n3)c2)cc1 ; c1ccc(-c2cccc(-c3cc(-c4ccc5c(c4)c4ccccc4c4cc6[se]c7ccccc7c6cc54)nc(-c4ccccc4)n3)c2)cc1 ; c1ccc(-c2cccc(-c3cc(-c4ccc5c(c4)c4ccccc4c4c5ccc5c6ccccc6[se]c54)nc(-c4ccccc4)n3)c2)cc1]

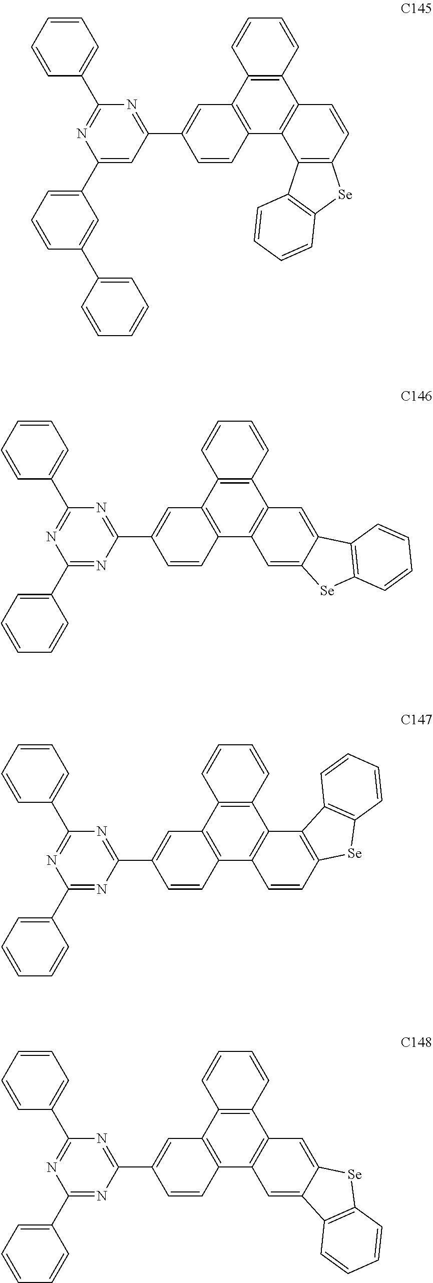 [SMILES: c1ccc(-c2cccc(-c3cc(-c4ccc5c(c4)c4ccccc4c4ccc6[se]c7ccccc7c6c45)nc(-c4ccccc4)n3)c2)cc1 ; c1ccc(-c2nc(-c3ccccc3)nc(-c3ccc4c(c3)c3ccccc3c3cc5c(cc43)[se]c3ccccc35)n2)cc1 ; c1ccc(-c2nc(-c3ccccc3)nc(-c3ccc4c(c3)c3ccccc3c3c4ccc4[se]c5ccccc5c43)n2)cc1 ; c1ccc(-c2nc(-c3ccccc3)nc(-c3ccc4c(c3)c3ccccc3c3cc5[se]c6ccccc6c5cc43)n2)cc1]

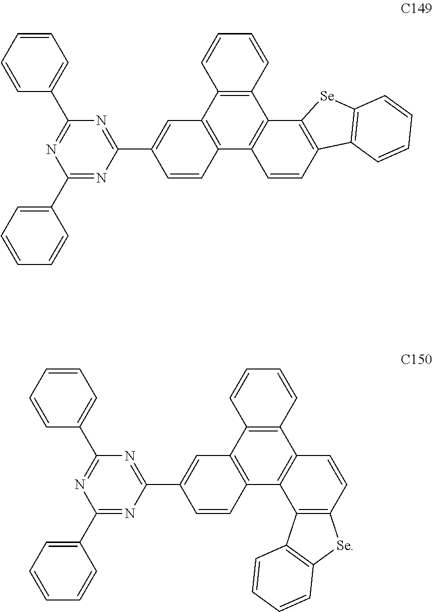

According to one or more aspects of the present disclosure, a red organic light emitting diode comprises a first electrode, a second electrode, and an organic thin film layer between the first electrode and the second electrode. The organic thin film layer is selected from the group consisting of a first light emitting layer, a first hole blocking layer, a first electron transport layer, and combinations thereof. The first light emitting layer comprises a first host and a first guest. The organic thin film layer comprises the organic compound of the present disclosure. An electroluminescence (EL) peak of the first guest is greater than about 600 nm.

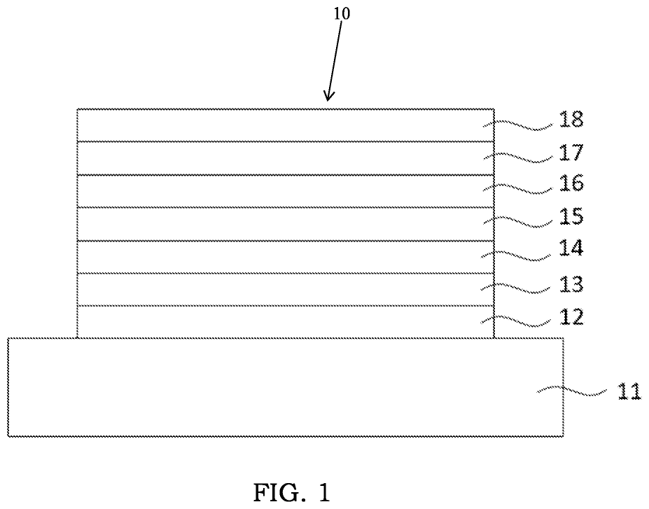

FIG. 1 shows a schematic cross-sectional view of a first embodiment of the red OLED of the present disclosure. Referring to FIG. 1, a red OLED 10 comprises a substrate 11, a first electrode 12, a hole injection layer 13, a hole transport layer 14, an electron blocking layer 15, a light emitting layer 16 and an electron transport layer 17. The first electrode 12 is on the substrate 11. The hole injection layer 13, the hole transport layer 14, and the electron blocking layer 15 are formed on the first electrode 12. The light emitting layer 16 is formed on the electron blocking layer 15. The light emitting layer 16 comprises a guest and a host. Each of the host and the guest may comprise an organic compound as a material. The guest accounts for 1% to 10% of the volume of the light emitting layer 16. The host material may be selected from the above-mentioned organic compounds of the present disclosure. The host material accounts for 90% to 99% of the volume of the light emitting layer 16. The electron transport layer 17 is formed on the light emitting layer 16. The material of the electron transport layer 16 may also comprise an organic compound selected from the above-mentioned organic compounds of the present disclosure. The second electrode 18 is formed on the electron transport layer 17. At least one of the first electrode 12 and the second electrode 18 may be transparent or semi-transparent for the emitted light to penetrate. An electroluminescence (EL) peak of the guest is greater than about 600 nm.

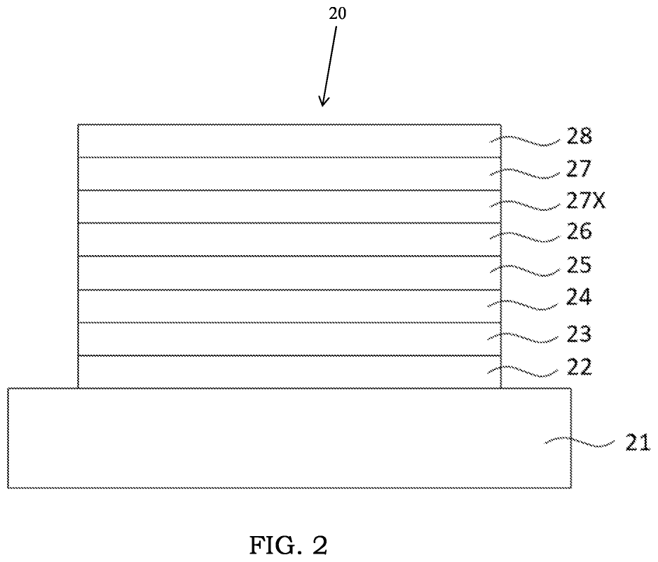

FIG. 2 shows a schematic cross-sectional view of a second embodiment of the red OLED of the present disclosure. Referring to FIG. 2, a red OLED 20 comprises a substrate 21, a first electrode 22, a hole injection layer 23, a hole transport layer 24, an electron blocking layer 25, a light emitting layer 26, a hole blocking layer 27X, an electron transport layer 27 and a second electrode 28. The first electrode 22 is on the substrate 21. The hole injection layer 23, the hole transport layer 24, the electron blocking layer 25 and the light emitting layer 26 are formed on the first electrode 22. A light emitting layer 26 is formed on the electron blocking layer 25.

The light emitting layer 26 comprises a guest and a host. Each of the host and the guest may comprise an organic compound as a material. The guest accounts for 1% to 10% of the volume of the light emitting layer 26. The host material may be selected from the above-mentioned organic compounds of the present disclosure. The host material accounts for 90% to 99% of the volume of the light emitting layer 26. The hole blocking layer 27X is formed on the light emitting layer 26. The electron transport layer 27 is formed on the light emitting layer 26. An electron transport layer 27 is formed on the hole blocking layer 27X. The material of the electron transport layer 27, the material of the hole blocking layer 27X, and the host material, may be the same or similar with each other, and mat be all selected from the organic compounds of the present disclosure. The second electrode 28 is formed on the electron transport layer 27. At least one of the first electrode 22 and the second electrode 28 may be transparent or semi-transparent for the emitted light to penetrate. An electroluminescence (EL) peak of the guest is greater than about 600 nm.

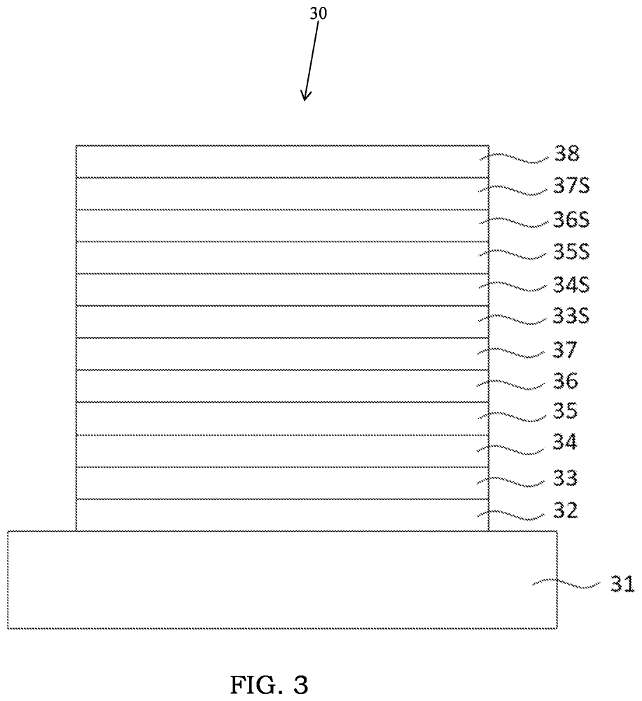

FIG. 3 shows a schematic cross-sectional view of a third embodiment of the red OLED of the present disclosure. Referring to FIG. 3, the red OLED 30 comprises a substrate 31, a first electrode 32, a first hole injection layer 33, a first hole transport layer 34, a first electron blocking layer a 35, a first light emitting layer 36, a first electron transport layer 37, a second hole injection layer 33S, a second hole transport layer 34S, a second electron blocking layer 35S, a second light emitting layer 36S, a second electron transport layer 37S and a second electrode 38. The first light emitting layer 36 is formed on the first electron blocking layer 35. The first electrode 32 is on the substrate 31. The first hole injection layer 33, the first hole transport layer 34, the first electron blocking layer 35 and the first light emitting layer 36 are formed on the first electrode 32. The first light emitting layer 36 comprises a first guest and a first host. Each of the first host and the first guest may comprise an organic compound as a material. The first guest accounts for 1% to 10% of the volume of the first light emitting layer 36. The first host material may be selected from the above-mentioned organic compounds of the present disclosure. The first host material accounts for 90% to 99% of the volume of the first light emitting layer 36. The first electron transport layer 37 is formed on the first light emitting layer 36. The second hole injection layer 33S, the second hole transport layer 34S, and the second electron blocking layer 35S are formed on the first electron transport layer 37. The second light emitting layer 36S is formed on the second electron blocking layer 35S. The second light emitting layer 36S comprises a second guest and a second host. Each of the second host and the second guest may comprise an organic compound as a material. The second guest accounts for 1% to 10% of the volume of the second light emitting layer 36S. The second host material may be selected from the organic compounds of the present disclosure (which can be the same or similar to the first host material). The second host material accounts for 90% to 99% of the volume percentage of the second light emitting layer 36S. The second electron transport layer 37S is formed on the second light emitting layer 36S. The material of the second electron transport layer 37S can also be selected from the organic compounds of the present disclosure (which can be the same or similar to the first host material). The second electrode 38 is formed on the second electron transport layer 37S. At least one of the first electrode 32 and the second electrode 38 may be transparent or semi-transparent for the emitted light to penetrate. An electroluminescence (EL) peak of the first guest is greater than about 600 nm. An electroluminescence (EL) peak of the second guest is greater than about 600 nm.

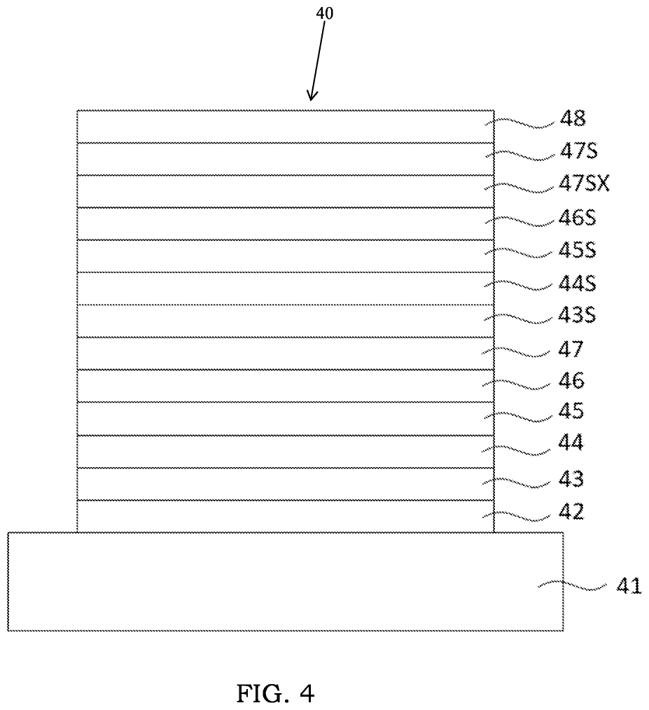

FIG. 4 shows a schematic cross-sectional view of a fourth embodiment of the red OLED of the present disclosure. Referring to FIG. 4, the red OLED 40 comprises a substrate 41, a first electrode 42, a first hole injection layer 43, a first hole transport layer 44, a first electron blocking layer 45, a first light emitting layer 46, a first electron transport layer 47, a second hole injection layer 43S, a second hole transport layer 44S, a second electron blocking layer 45S, a second light emitting layer 46S, a hole blocking layer 47SX, a second electron transport layer 47S and a second electrode 48. The first light emitting layer 46 is formed on the first electron blocking layer 45. The first electrode 42 is on the substrate 41. The first hole injection layer 43, the first hole transport layer 44, the first electron blocking layer 45 and the first light emitting layer 46 are formed on the first electrode 42. The first light emitting layer 46 comprises a first guest and a first host. Each of the first host and the first guest may comprise an organic compound as a material. The first guest accounts for 1% to 10% of the volume of the first light emitting layer 46. The first host material may be selected from the above-mentioned organic compounds of the present disclosure. The first host material accounts for 90% to 99% of the volume of the first light emitting layer 46. The first electron transport layer 47 is formed on the first light emitting layer 46. The second hole injection layer 43S, the second hole transport layer 44S, and the second electron blocking layer 45S are formed on the first electron transport layer 47. The second light emitting layer 46S is formed on the second electron blocking layer 45S. The second light emitting layer 46S comprises a second guest and a second host. Each of the second host and the second guest may comprise an organic compound as a material. The second guest accounts for 1% to 10% of the volume of the second light emitting layer 46S. The second host material may be selected from the organic compounds of the present disclosure (which can be the same or similar to the first host material). The second host material accounts for 90% to 99% of the volume percentage of the second light emitting layer 46S. The hole blocking layer 47SX and the second electron transport layer 47S are sequentially formed on the second light emitting layer 46S. The material of the second electron transport layer 47S can also be selected from the organic compounds of the present disclosure (which can be the same or similar to the first host material). The second electrode 48 is formed on the second electron transport layer 47S. At least one of the first electrode 42 and the second electrode 48 may be transparent or semi-transparent for the emitted light to penetrate.

Referring to Table 1 and Table 2, compared to prior art, the inventive device examples of the present invention disclose red OLEDs having higher device efficiencies (for example but not limited to current efficiency or luminous efficiency), or having higher lifetime (LT₉₅ represents lifetime in Table 1 and Table 2). LT₉₅ value is defined as the time required for the brightness level to drop to 95% relative to the initial brightness, which is used as a measure of the lifetime of an OLED.

Referring to Table 1 and Table 2. The host material of the light emitting layer, and the material of the electron transport layer, may be formed of the same or similar material. If doing so, the electrons are transported faster, and the interface between the materials is more stable. The fast transportation and stable interface may be helpful for improved efficiency and/or for extended lifetime. Alternatively, if the host material of the light emitting layer, and the material of the electron blocking layer, are formed of the same material or similar material, it is also helpful for improved efficiency and/or for extended lifetime.

TABLE 1

| | | Current | Luminous | |
|---|---|---|---|---|
| | Voltage | efficiency | efficiency | LT₉₅ |
| Example | (V) | (cd/A) | (lm/W) | (hrs) |
| Comparative Example 1 | 4.7 | 8.6 | 5.7 | 58 |
| Inventive Device Example 1 | 4.5 | 9.8 | 6.8 | 85 |
| Inventive Device Example 2 | 4.4 | 10.3 | 7.4 | 91 |
| Inventive Device Example 3 | 4.5 | 9.5 | 6.6 | 78 |
| Inventive Device Example 4 | 4.5 | 9.6 | 6.7 | 65 |
| Inventive Device Example 5 | 4.4 | 10.5 | 7.5 | 108 |
| Inventive Device Example 6 | 4.4 | 10.6 | 7.6 | 113 |
| Inventive Device Example 7 | 4.5 | 10.3 | 7.2 | 98 |
| Inventive Device Example 8 | 4.3 | 11 | 8.0 | 110 |
| Inventive Device Example 9 | 4.3 | 10.7 | 7.8 | 101 |
| Inventive Device Example 10 | 4.3 | 11.3 | 8.3 | 129 |
| Inventive Device Example 11 | 4.3 | 11.4 | 8.3 | 138 |
| Inventive Device Example 12 | 4.4 | 10.8 | 7.7 | 110 |
| Inventive Device Example 13 | 4.4 | 11.1 | 7.9 | 115 |

TABLE 2

| | | Current | Luminous | |
|---|---|---|---|---|
| | | efficiency | efficiency | |
| | Voltage | (cd/A) | (lm/W) | LT₉₅ |
| | (V) | Current | Luminous | (hrs) |
| Example | Voltage | efficiency | efficiency | LT₉₅ |
| Example | (V) | (cd/A) | (lm/W) | (hrs) |
| Comparative Example 2 | 7.8 | 16.8 | 6.8 | 128 |
| Inventive Device Example 14 | 7.4 | 19.2 | 8.1 | 197 |
| Inventive Device Example 15 | 7.3 | 19.5 | 8.4 | 210 |
| Inventive Device Example 16 | 7.3 | 20.2 | 8.7 | 235 |
| Inventive Device Example 17 | 7 | 21.6 | 9.7 | 245 |
| Inventive Device Example 18 | 7.2 | 19.5 | 8.5 | 212 |
| Inventive Device Example 19 | 7.1 | 22 | 9.7 | 280 |
| Inventive Device Example 20 | 7 | 22.3 | 10.0 | 306 |
| Inventive Device Example 21 | 7.3 | 20.5 | 8.8 | 230 |
| Inventive Device Example 22 | 7.2 | 20.7 | 9.0 | 278 |

### PRODUCTION OF COMPARATIVE EXAMPLE 1

An ITO glass, having a thickness of about 1250 angstroms (Å) and a sheet resistance of 15 ohms/unit area, servs as an ITO glass substrate. Before loading the pre-patterned ITO glass substrate into an vacuum evaporation chamber, the surface of the ITO glass substrate is cleaned with a cleaning agent, is then baked, and is irradiate with ultraviolet ozone. The ITO glass substrate is transferred to the vacuum evaporation chamber, to form an ITO electrode as a first electrode on the ITO glass substrate. A plurality of compounds, as shown in paragraph [0048], are evaporated and respectively formed organic layers on the first electrode. Finally, an upper metal is formed to serves as a second electrode on the organic layers.

By a heated vapor deposition source at a vacuum of about 10⁻⁶ Torr, the compounds, shown in paragraph [0048], are sequentially vapor-deposited to form a hole injection layer (HIL) with a thickness of about 200 angstroms (Å), a hole transport layer (HTL) with a thickness of about 1700 angstroms (Å), and an electron blocking layer (EBL) with a thickness of 200 angstroms (Å). A light emitting layer is subsequently formed with a thickness of about 300 angstroms (Å) on the electron blocking layer (EBL). The light emitting layer comprises a host material and a guest material. The host material is co-evaporated by compounds Host1 and Host2 at a ratio of 6:4. The host material accounts for 95% of the volume of the light emitting layer. The guest PT-238I accounts for 5% of the volume of the light emitting layer. PT-238I is a product name of the applicant (LUMINESCENCE TECHNOLOGY CORP.). By having a guest PT-238I, a maximum wavelength of a photoluminescence (PL) peak of a light emitting layer may approach to about 623 nm. Compounds ETL and LiQ is then co-evaporated at a ratio of 1:1 to form an electron transport layer (ETL having LiQ) having a thickness of about 250 angstroms (Å) on the on the light emitting layer. Finally, Al is plated with a thickness of 1000 about angstroms (Å) as a second electrode. Subsequently, the OLED is transferred from the evaporation chamber to the drying box, and is packaged with a UV curable glue and a glass cover plate containing a moisture absorbent. The OLED emits red light with a light emitting area of 0.25 square centimeters. In Table 1, to characterize the OLED, the voltage, current efficiency, and luminous efficiency are measured at 1000 nits by a constant current source (KEITHLEY 2400) and a photometer (PHOTO RESEARCH SpectraScan PR 650) at room temperature. The lifetime LT₉₅ at a constant brightness of about 4000 nits is also measured and listed in Table 1. The LT₉₅ value is defined as the time required for the brightness level to drop to 95% relative to the initial brightness, which is used as a measure of the lifetime of an OLED.

### PRODUCTION OF INVENTIVE DEVICE EXAMPLE 1

Similarly, referring to the production parameters of Comparative Example 1, to produce Inventive Device Example 1, except that the dual host material of co-evaporated compounds Host1 and Host2 in Comparative Example 1 is replaced with a single host material (an organic compound C31). The other conditions are the same as Comparative Example 1.

### PRODUCTION OF INVENTIVE DEVICE EXAMPLE 2

Referring to the production parameters of Comparative Example 1, to produce Inventive Device Example 2, except that the dual host material of co-evaporated compounds Host1 and Host2 in Comparative Example 1 is replaced with a single host material (an organic compound C21). The other conditions are the same as Comparative Example 1.

### PRODUCTION OF INVENTIVE DEVICE EXAMPLE 3

Referring to the production parameters of Comparative Example 1, to produce Inventive Device Example 3, except that the dual host material of co-evaporated compounds Host1 and Host2 in Comparative Example 1 is replaced with a single host material (an organic compound C96). The other conditions are the same as Comparative Example 1.

### PRODUCTION OF INVENTIVE DEVICE EXAMPLE 4

Referring to the production parameters of Comparative Example 4, to produce Inventive Device Example 4, except that the dual host material of co-evaporated compounds Host1 and Host2 in Comparative Example 1 is replaced with a single host material (an organic compound C56). The other conditions are the same as Comparative Example 1.

### PRODUCTION OF INVENTIVE DEVICE EXAMPLE 5

Referring to the production parameters of Inventive Device Example 2, to produce Inventive Device Example 5, except that the material of ETL in Inventive Device Example 2 is replaced with an organic compound C21. The other conditions are the same as Inventive Device Example 2.

### PRODUCTION OF INVENTIVE DEVICE EXAMPLE 6

Referring to the production parameters of Inventive Device Example 5, to produce Inventive Device Example 6, except that a hole blocking layer comprising an organic compound C21 as a material is formed with a thickness of about 50 angstroms (Å) between the light emitting layer and the ETL. The other conditions are the same as Inventive Device Example 5.

### PRODUCTION OF INVENTIVE DEVICE EXAMPLE 7

Referring to the production parameters of Comparative Example 1, to produce Inventive Device Example 7, except that the dual host material of co-evaporated compounds Host1 and Host2 in Comparative Example 1 is replaced with a single host material (an organic compound C26). The other conditions are the same as Comparative Example 1.

### PRODUCTION OF INVENTIVE DEVICE EXAMPLE 8

Referring to the production parameters of Comparative Example 1, to produce Inventive Device Example 8, except that the dual host material of co-evaporated compounds Host1 and Host2 in Comparative Example 1 is replaced with a single host material (an organic compound C1). The other conditions are the same as Comparative Example 1.

### PRODUCTION OF INVENTIVE DEVICE EXAMPLE 9

Referring to the production parameters of Comparative Example 1, to produce Inventive Device Example 9, except that the dual host material of co-evaporated compounds Host1 and Host2 in Comparative Example 1 is replaced with a single host material (an organic compound C11). The other conditions are the same as Comparative Example 1.

### PRODUCTION OF INVENTIVE DEVICE EXAMPLE 10

Referring to the production parameters of Inventive Device Example 8, to produce Inventive Device Example 10, except that the material of ETL in Comparative Example 8 is replaced with an organic compound C1. The other conditions are the same as Inventive Device Example 8.

### PRODUCTION OF INVENTIVE DEVICE EXAMPLE 11

Referring to the production parameters of Inventive Device Example 10, to produce Inventive Device Example 11, except that a hole blocking layer comprising an organic compound C21 as a material is formed with a thickness of about 50 angstroms (Å) between the light emitting layer and the ETL. The other conditions are the same as Inventive Device Example 10.

### PRODUCTION OF INVENTIVE DEVICE EXAMPLE 12

Referring to the production parameters of Inventive Device Example 9, to produce Inventive Device Example 12, except that the material of ETL in Inventive Device Example 9 is replaced with an organic compound C1. The other conditions are the same as Inventive Device Example 9.

### PRODUCTION OF INVENTIVE DEVICE EXAMPLE 13

Referring to the production parameters of Inventive Device Example 12, to produce Inventive Device Example 13, except that a hole blocking layer comprising an organic compound C11 as a material is formed with a thickness of about 50 angstroms (Å) between the light emitting layer and the ETL. The other conditions are the same as Inventive Device Example 12.

As shown in Table 1, by replacing the host material with organic compound C31, the efficiency of the OLED can be improved from 8.6 cd/A in Comparative Example 1 to about 9.8 cd/A in Inventive Device Example 1. Also in Inventive Device Example 1, the operating voltage is reduced to about 0.2V. The LT₉₅ is extended to about 85 hours. It is shown that organic compound C31 is a single host material having characteristics for better red light. The single host material is more stable to be controlled in a vapor deposition process than a conventional dual host material. That is, such OLED having organic compound C31 as the host material reduces the voltage, increases the current efficiency, and/or extend the lifetime of the OLED.

Similar reduction, increase and/or extension are also shown in Inventive Device Examples 2-4, 7-9 and 11. In each of Inventive Device Examples 2, 8 and 9, the ETL is replaced, a HBL with a thickness of about 50 angstrom (Å) is added. It can be found that the voltage, luminous efficiency and/or L₉₅ of the red OLEDs (Inventive Device Examples 2, 8 and 9) are further improved.

The performance of Inventive Device Example 11 is the most preferable, the current efficiency can reach about 11.4 cd/A, the operating voltage is reduced to about 0.4V, and the LT₉₅ is extended to about 138 hours. Compared with Comparative Example 1, the improvement of Inventive Device Example 11 in performance is quite significant.

### PRODUCTION OF COMPARATIVE EXAMPLE 2

An ITO glass, having a thickness of about 1250 angstroms (Å) and a sheet resistance of 15 ohms/unit area, servs as an ITO glass substrate. Before loading the pre-patterned ITO glass substrate into an vacuum evaporation chamber, the surface of the ITO glass substrate is cleaned with a cleaning agent, is then baked, and is irradiate with ultraviolet ozone. The ITO glass substrate is transferred to the vacuum evaporation chamber, to form an ITO electrode as a first electrode on the ITO glass substrate. A plurality of compounds, as shown in paragraph [0048], are evaporated and respectively formed organic layers on the first electrode. Finally, an upper metal is formed to serves as a second electrode on the organic layers.

By a heated vapor deposition source at a vacuum of about 10⁻⁶ Torr, the compounds, shown in paragraph [0048], are sequentially vapor-deposited to form a first hole injection layer (HIL) with a thickness of about 200 angstroms (Å), a first hole transport layer (HTL) with a thickness of about 1700 angstroms (Å), and a first electron blocking layer (EBL) with a thickness of 120 angstroms (Å). A first light emitting layer is subsequently formed with a thickness of about 300 angstroms (Å) on the first electron blocking layer (EBL). The first light emitting layer comprises a host material and a guest material. The host material is co-evaporated by compounds Host1 and Host2 at a ratio of 6:4. The host material accounts for 95% of the volume of the light emitting layer. The guest PT-238I accounts for 5% of the volume of the light emitting layer. Compounds ETL and LiQ is then co-evaporated at a ratio of 2:0.1 to form a first electron transport layer (ETL having LiQ) having a thickness of about 250 angstroms (Å) on the on the first light emitting layer. The compounds, shown in paragraph [0048], are sequentially vapor-deposited to form a second hole injection layer (HIL) with a thickness of about 200 angstroms (Å), a second hole transport layer (HTL) with a thickness of about 450 angstroms (Å), and a second electron blocking layer (EBL) with a thickness of 120 angstroms (Å). A second light emitting layer is subsequently formed with a thickness of about 300 angstroms (Å) on the second electron blocking layer (EBL). The second light emitting layer comprises a host material and a guest material. The host material is co-evaporated by compounds Host1 and Host2 at a ratio of 6:4. The host material accounts for 95% of the volume of the light emitting layer. The guest PT-238I accounts for 5% of the volume of the light emitting layer. Compounds ETL and LiQ is then co-evaporated at a ratio of 1:1 to form a second electron transport layer (ETL having LiQ) having a thickness of about 250 angstroms (Å) on the on the second light emitting layer. Finally, Al is plated with a thickness of 1000 about angstroms (Å) as a second electrode. Subsequently, the OLED is packaged and is characterized in Table 2. The voltage, current efficiency, and luminous efficiency are measured at 1000 nits. The lifetime LT₉₅ at a constant brightness of about 4000 nits is also measured and listed in Table 2.

### PRODUCTION OF INVENTIVE DEVICE EXAMPLE 14

Referring to the production parameters of Comparative Example 2, to produce Inventive Device Example 14, except that the dual host material of co-evaporated compounds Host1 and Host2 in Comparative Example 2 is replaced with a single host material (an organic compound C21). The other conditions are the same as Comparative Example 2.

### PRODUCTION OF INVENTIVE DEVICE EXAMPLE 15

Referring to the production parameters of Inventive Device Example 14, to produce Inventive Device Example 15, except that the material of the second ETL in Inventive Device Example 14 is replaced with an organic compound C21. The other conditions are the same as Inventive Device Example 14.

### PRODUCTION OF INVENTIVE DEVICE EXAMPLE 16

Referring to the production parameters of Inventive Device Example 15, to produce Inventive Device Example 16, except that a hole blocking layer comprising an organic compound C21 as a material is formed with a thickness of about 50 angstroms (Å) between the second light emitting layer and the second ETL. The other conditions are the same as Inventive Device Example 15.

### PRODUCTION OF INVENTIVE DEVICE EXAMPLE 17

Referring to the production parameters of Comparative Example 2, to produce Inventive Device Example 17, except that the dual host material of co-evaporated compounds Host1 and Host2 in Comparative Example 2 is replaced with a single host material (an organic compound C1). The other conditions are the same as Comparative Example 2.

### PRODUCTION OF INVENTIVE DEVICE EXAMPLE 18

Referring to the production parameters of Comparative Example 2, to produce Inventive Device Example 18, except that the dual host material of co-evaporated compounds Host1 and Host2 in Comparative Example 2 is replaced with a single host material (an organic compound C11). The other conditions are the same as Comparative Example 2.

### PRODUCTION OF INVENTIVE DEVICE EXAMPLE 19

Referring to the production parameters of Inventive Device Example 17, to produce Inventive Device Example 19, except that the material of the second ETL in Comparative Example 8 is replaced with an organic compound C1. The other conditions are the same as Inventive Device Example 17.

### PRODUCTION OF INVENTIVE DEVICE EXAMPLE 20

Referring to the production parameters of Inventive Device Example 19, to produce Inventive Device Example 20, except that a hole blocking layer comprising an organic compound C1 as a material is formed with a thickness of about 50 angstroms (Å) between the second light emitting layer and the second ETL. The other conditions are the same as Inventive Device Example 19.

### PRODUCTION OF INVENTIVE DEVICE EXAMPLE 21

Referring to the production parameters of Inventive Device Example 18, to produce Inventive Device Example 21, except that the material of the second ETL in Comparative Example 8 is replaced with an organic compound C11. The other conditions are the same as Inventive Device Example 18.

### PRODUCTION OF INVENTIVE DEVICE EXAMPLE 22

Referring to the production parameters of Inventive Device Example 21, to produce Inventive Device Example 22, except that a hole blocking layer comprising an organic compound C11 as a material is formed with a thickness of about 50 angstroms (Å) between the second light emitting layer and the second ETL. The other conditions are the same as Inventive Device Example 21.

As shown in Table 2, by replacing the host material with an organic compound C21 of Inventive Device Example 14, the efficiency of the OLED can be improved from 16.8 cd/A in Comparative Example 2 to about 19.2 cd/A in Inventive Device Example 14. Also in Inventive Device Example 1, the operating voltage is reduced to about 0.4V. The LT₉₅ is extended to about 197 hours. It is shown that organic compound C21 is a single host material having characteristics for better red light. The single host material is more stable to be controlled in a vapor deposition process than a conventional dual host material. That is, such OLED having organic compound C21 as the host material reduces the voltage, increases the current efficiency, and/or extend the lifetime of the OLED.

Similar reduction, increase and/or extension are also shown in Inventive Device Examples 17 and 18. In each of Inventive Device Examples 17 and 18, the ETL is replaced, a HBL with a thickness of about 50 angstrom (Å) is added. It can be found that the voltage, luminous efficiency and/or L₉₅ of the red OLEDs (Inventive Device Examples 17 and 18) are further improved.

The performance of Inventive Device Example 11 is the most preferable, the current efficiency can reach about 11.4 cd/A, the operating voltage is reduced to about 0.4V, and the LT₉₅ is extended to about 138 hours. Compared with Comparative Example 1, the improvement of Inventive Device Example 11 in performance is quite significant.

When non-obviousness of the present disclosure is evaluated, the technical solution of the invention cannot be required to produce an advantageous technical effect in any situation and in all aspects. Such requirement does not comply with non-obviousness-related provisions of a patent law.

One person having ordinary skill in the art of the present application, in actual use, may select a material of a compound to take advantage of one kind of luminescent data (for example, to emit a specific color of light). In the same art of the present application, however, it is not always necessary for the present invention to take advantage of other kinds of luminescent data such as an operating voltage, current efficiency or lifetime of the device.

When the non-obviousness of the present disclosure is evaluated, it shall not be required to take advantage of all kinds of luminescent data. As long as the present invention takes advantage of one kind of luminescent data, such as a lower operating voltage, a higher current efficiency or a longer lifetime, the device of the present disclosure shall be regarded as producing an advantageous luminescent effect. It shall not be required to have a general improvement of all kinds of luminescent data of the compound in any case. Moreover, the present disclosure shall be considered as a whole. The technical effect brought by the whole technical solution should not be negated, even if some luminescent data of the compound are not good, or one luminescent data is not good for some kinds of color of light or for the application of some kinds of host.

A compound of the present application, as a material, shall not be required to improve all kinds of luminesce data, for all kinds of color of light, in the case of application of all kinds of host. As long as one kind of luminesce data, such as a current efficiency or lifetime of a specific color of light, is improved in the case of a specific host, the present invention shall be regarded as producing an advantageous technical effect. The advantageous technical effect is non-obvious enough to be a prominent substantive feature, so that the corresponding technical solution of the present disclosure involves an inventive step.

In some cases, the red OLED is a lighting panel or a backlight panel.

Detailed preparation of the organic compounds of the present disclosure ise clarified by exemplary embodiments, but the present invention is not limited thereto. Preparation Examples 1-5 show the preparation of the organic compounds of the present disclosure, and Inventive Device Examples 1-22 show the production of the OLEDs.

### PREPARATION EXAMPLE 1

### Synthesis of 1-bromo-2-iodo-4-methoxybenzene

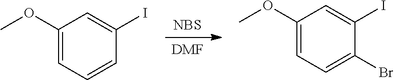

A mixture of 40 g (171 mmol) of 1-iodo-3-methoxybenzene, 32 g (179 mmol) of N-bromosuccinimide, and 600 ml of DMF was degassed and placed under nitrogen, and then heated at 80° C. for 12 hrs. After the reaction finished, the mixture was allowed to cool to room temperature. Subsequently, the solvent was removed under reduced pressure, and the crude product was purified by column chromatography, yielding 45 g of 1-bromo-2-iodo-4-methoxybenzene as yellow oil (84.1%). ¹H NMR (CDCl₃, 400 MHz): chemical shift (ppm) 7.43(dd, 1H), 7.35(dd, 1H), 6.73(dd, 1H), 3.74(s, 3H).

### Synthesis of 2-bromo-5-methoxy-1,1′-biphenyl

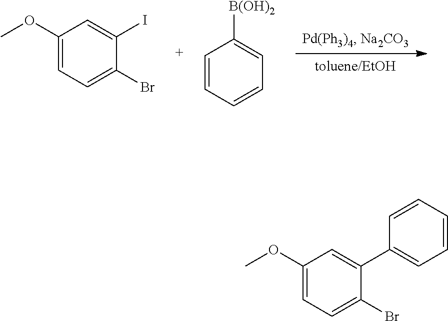

A mixture of 40 g (127.8 mmol) of 1-bromo-2-iodo-4-methoxybenzene, 15.6 g (127.8 mmol) of phenylboronic acid, 2.95 g (2.56 mmol) of Pd(Ph₃)₄, 155 ml of 2M Na₂CO₃, 100 ml of EtOH and 300 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 30 g of 2-bromo-5-methoxy-1,1′-biphenyl as colorless liquid (89.2%). ¹H NMR (CDCl₃, 400 MHz): chemical shift (ppm) 7.55(d, 1H), 7.46-7.38 (m, 5H), 6.89(d, 1H), 6.79(dd, 1H), 3.81(s, 3H).

### Synthesis of (5-methoxy-[1,1′-biphenyl]-2-yl) boronic acid

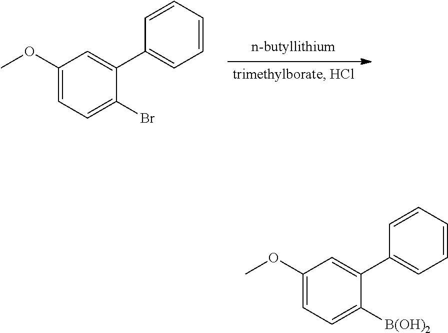 [SMILES: COc1ccc(Br)c(-c2ccccc2)c1 ; "n-butyllithium" ; "trimethylborate, HCl" ; COc1ccc(B(O)O)c(-c2ccccc2)c1]

The compound 2-bromo-5-methoxy-1,1′-biphenyl (30 g, 114 mmol) was mixed with 600 ml of dry THF. To the mixture, 54.7 ml of N-butyllithium (137 mmol) was added at −60° C. and the mixture was stirred for 1 hrs. After the reaction finished, 17.8 g (171 mmol) of trimethyl borate was added and the mixture was stirred overnight. 228 ml (228 mmole) of 1M HCl was added and the mixture was stirred for 1 hrs. The mixture was extracted with ethyl acetate/H₂O, and the organic layer was removed under reduced pressure. The crude product was washed by hexane, yielding 19.5 g of (5-methoxy-[1,1′-biphenyl]-2-yl) boronic acid as white solid (75%).

### Synthesis of 3-(5-methoxy-[1,1′-biphenyl]-2-yl)dibenzo [b,d]-thiophene

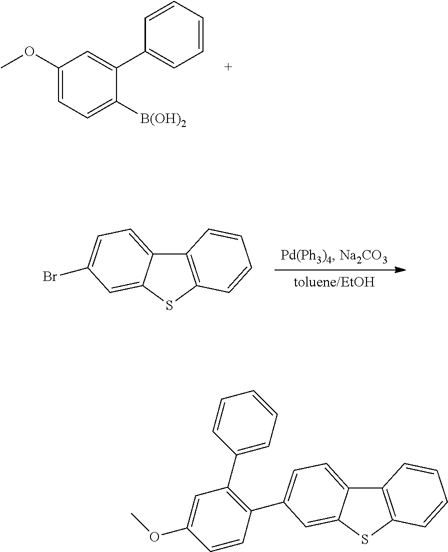

A mixture of 20 g (87.7 mmol) of (5-methoxy-[1,1′-biphenyl]-2-yl)-boronic acid, 25.4 g (96.5 mmol) of 3-bromodibenzo[b,d]thiophene, 2.03 g (1.75 mmol) of Pd(Ph₃)₄, 87.7 ml of 2M Na₂CO₃, 200 ml of EtOH and 400 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 23.1 g of 3-(5-methoxy-[1,1′-biphenyl]-2-yl)-dibenzo[b,d]thiophene as white solid (71.9%). ¹H NMR (CDCl₃, 400 MHz): chemical shift (ppm) 8.47(d, 1H), 8.12-8.06 (m, 3H), 8.01 (d, 1H), 7.77-7.74 (m, 3H), 7.49-7.45 (m, 4H), 7.41-7.38 (m, 2H), 7.02 (d, 1H), 3.81 (s, 3H).

### Synthesis of 6-methoxybenzo[b]triphenyleno[2,3-d]thiophene

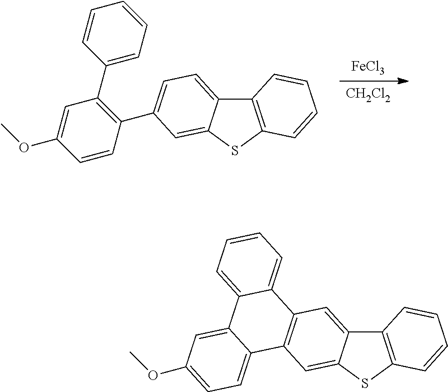

The compound 3-(5-methoxy-[1,1′-biphenyl]-2-yl)dibenzo[b,d]-thiophene (20 g, 54.6 mmol) was mixed with 700 ml of CH₂Cl₂. To the mixture, 88.5 g of FeCl₃(546 mmol) was added and the mixture was stirred for 1 hrs. After the reaction finished, the solvent was removed under reduced pressure, and the crude product was purified by column chromatography, yielding 8.5 g of 6-methoxybenzo[b]triphenyleno[2,3-d]-thiophene as white solid (42.7%). ¹H NMR (CDCl₃, 400 MHz): chemical shift (ppm) 8.91-8.89 (m, 2H), 8.81 (d, 1H), 8.49 (d, 1H), 8.14 (m, 2H), 7.99 (d, H), 7.89-7.85 (m, 2H), 7.62 (s, 1H), 7.54-7.51 (m, 2H), 7.36 (d, 1H), 3.82 (s, 3H).

### Synthesis of benzo[b]triphenyleno[2,3-d]thiophen-6-ol

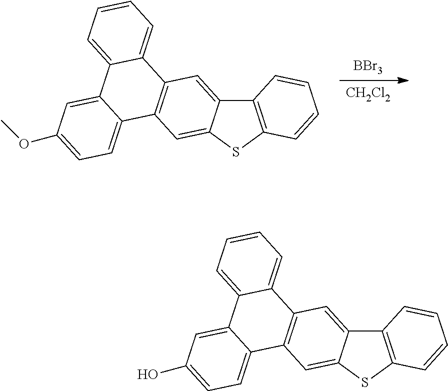

The compound 6-methoxybenzo[b]triphenyleno[2,3-d]-thiophene (10 g, 27.4 mmol) was mixed with 400 ml of CH₂Cl₂. To the mixture, 8.25 g of BBr₃(32.9 mmol) was added and the mixture was stirred overnight. After the reaction finished, the solvent was removed under reduced pressure, and the crude product was purified by column chromatography, yielding 8.8 g of benzo[b]triphenyleno[2,3-d]thiophen-6-ol as white solid (91.5%). ¹H NMR (CDCl₃, 400 MHz): chemical shift (ppm) 8.89-8.87 (m, 2H), 8.78 (d, 1H), 8.45 (d, 1H), 8.09 (m, 2H), 7.94 (d, H), 7.86-7.83 (m, 2H), 7.58 (s, 1H), 7.51-7.48 (m, 2H), 7.31 (d, 1H), 5.41 (s, 1H).

### Synthesis of benzo[b]triphenyleno[2,3-d]thiophen-6-yl trifluoro-methanesulfonate

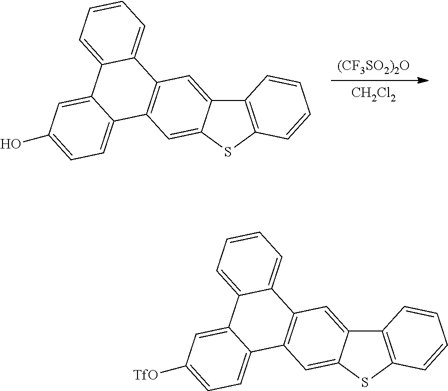

The compound benzo[b]triphenyleno[2,3-d]thiophen-6-ol (10 g, 28.5 mmol) was mixed with 450 ml of CH₂Cl₂. To the mixture, 3.4 g of pyridine (42.8 mmol) was added and the mixture was stirred for 1 hrs. To the mixture, 13.7 g of (CF₃SO₂)₂₀ (48.5 mmol) was added and the mixture was stirred for 1 hrs. After the reaction finished, the solvent was removed under reduced pressure, and the crude product was purified by column chromatography, yielding 10.5 g of benzo[b]triphenyleno[2,3-d]thiophen-6-yltrifluoro-methanesulfonate as yellow solid (55.9%). ¹H NMR (CDCl₃, 400 MHz): chemical shift (ppm) 8.99-8.95 (m, 3H), 8.47 (d, 1H), 8.14-8.11 (m, 3H), 7.97 (d, H), 7.88-7.85 (m, 2H), 7.58 (s, 1H), 7.53-7.51 (m, 2H).

### Synthesis of 2-(benzo [b]triphenyleno [2,3-d]thiophen-6-yl)-4,4,5,5-tetramethyl-1,3,2-dioxaborolane

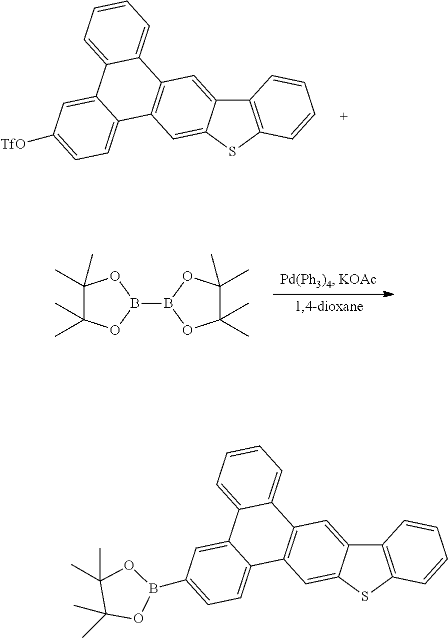

A mixture of 5 g (10.4 mmol) of benzo[b]triphenyleno[2,3-d]thiophen-6-yl trifluoromethanesulfonate, 3.16 g (12.4 mmol) of bis(pinacolato)diboron, 0.48 g (0.4 mmol) of Pd(Ph₃)₄, 2.04 g (20.8 mmol) of potassium acetate, and 60 ml of 1,4-dioxane was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 3.1 g of 2-(benzo [b]triphenyleno [2,3-d]thiophen-6-yl)-4,4,5,5-tetramethyl-1,3,2-dioxa borolane as white solid (65%). ¹H NMR (CDCl₃, 400 MHz): chemical shift (ppm) 8.94-8.88 (m, 3H), 8.47 (d, 1H), 8.15-8.12 (m, 3H), 7.99 (d,1H), 7.87-7.84 (m, 3H), 7.54-7.52 (m, 2H), 1.27 (s, 12H).

### Synthesis of 2-(benzo [b]triphenyleno [2,3-d]thiophen-6-yl)-4,6-diphenyl-1,3,5-triazine (Compound 5)

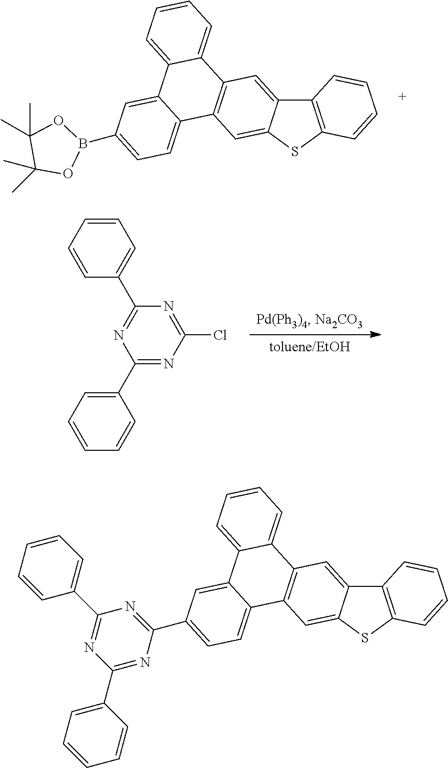

A mixture of 3 g (6.51 mmol) of 2-(benzo[b]triphenyleno[2,3-d]-thiophen-6-yl)-4,4,5,5-tetramethyl-1,3,2-dioxaborolane, 1.92 g (7.17 mmol) of 92-chloro-4,6-diphenyl-1,3,5-triazine, 0.15 g (0.13 mmol) of Pd(Ph₃)₄, 6.5 ml of 2M Na₂CO₃, 20 ml of EtOH and 40 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 2.5 g of 2-(benzo[b]triphenyleno[2,3-d]thiophen-6-yl)-4,6-diphenyl-1,3,5-triazine as yellow solid (68%). ¹H NMR (CDCl₃, 400 MHz): chemical shift (ppm)8.99-8.93 (m, 3H), 8.46 (d,1H), 8.33 (s,1H), 8.29-8.24 (m, 4H), 8.13-8.09 (m, 3H), 7.97 (d, H), 7.88-7.82 (m, 2H), 7.53-7.46 (m, 6H), 7.44-7.41 (m, 2H).

### PREPARATION EXAMPLES 2-5

We have used the same synthesis methods to get a series of the following compounds are synthesized analogously.

| Preparation Ex. | III | IV |
|---|---|---|
| | 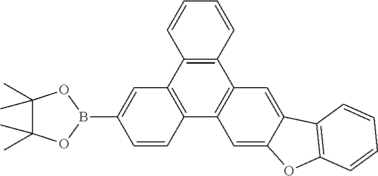 | 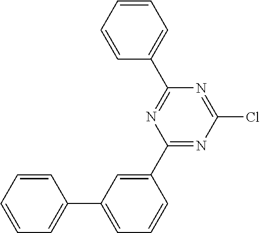 |
| | 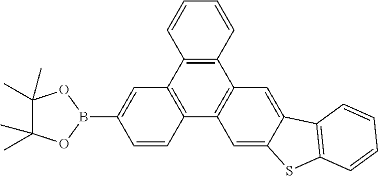 | 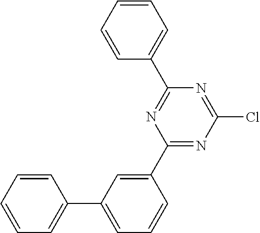 |
| | 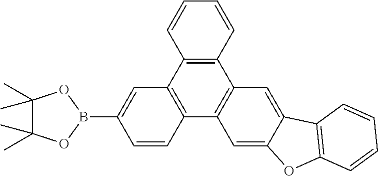 | 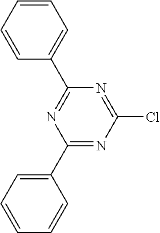 |
| | 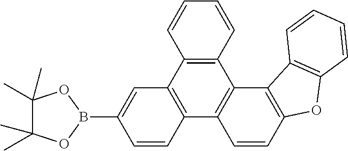 | 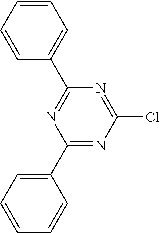 |

| Preparation Ex. | Product |
|---|---|
| | 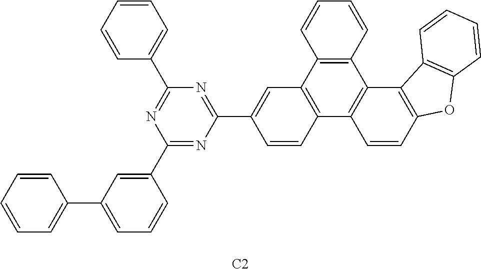 [SMILES: c1ccc(-c2cccc(-c3nc(-c4ccccc4)nc(-c4ccc5c(c4)c4ccccc4c4c5ccc5oc6ccccc6c54)n3)c2)cc1 ; "C2"] |
| | 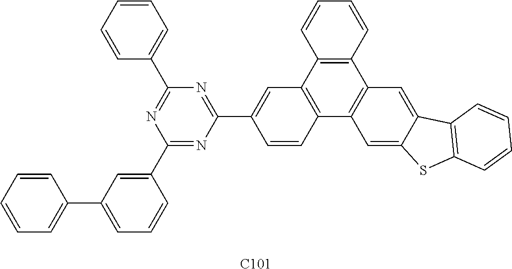 [SMILES: c1ccc(-c2cccc(-c3nc(-c4ccccc4)nc(-c4ccc5c(c4)c4ccccc4c4cc6c(cc54)sc4ccccc46)n3)c2)cc1 ; "C101"] |
| | 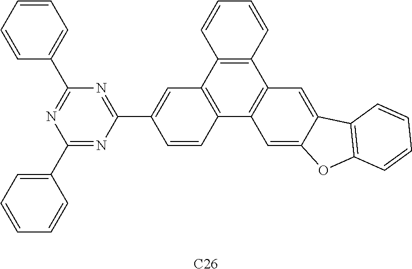 [SMILES: c1ccc(-c2nc(-c3ccccc3)nc(-c3ccc4c(c3)c3ccccc3c3cc5c(cc43)oc3ccccc35)n2)cc1 ; "C26"] |
| | 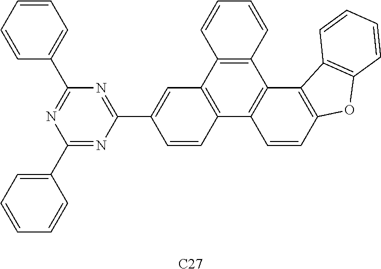 [SMILES: c1ccc(-c2nc(-c3ccccc3)nc(-c3ccc4c(c3)c3ccccc3c3c4ccc4oc5ccccc5c43)n2)cc1 ; "C27"] |

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. An organic compound represented by one of the following formula (1) to formula (5), or tautomers thereof: 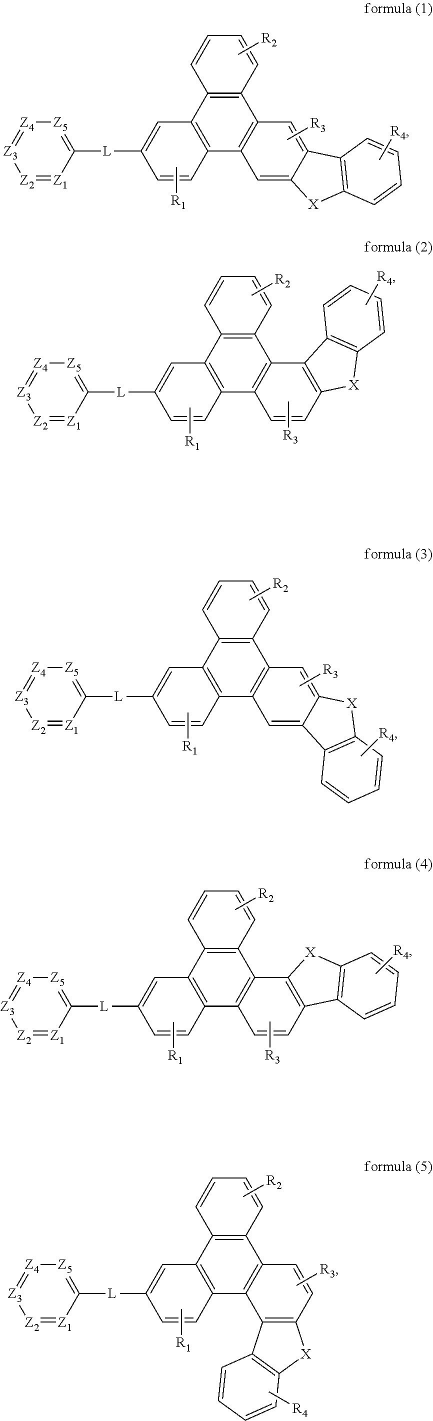
wherein X is selected from the group consisting of O, S, Se, NR₇ and SiR₅R₆;
where Z₁ to Z₅ is N or CR;
wherein at least one of Z₁ to Z₅ is N;
wherein R is selected from the group consisting of H, phenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof, wherein the S of the dibenzothienyl group in R is optionally changed to Se, and wherein the C connecting the dialkyl group in R is optionally changed to Si;
wherein L is a single bond, aryl or biphenyl;
wherein R₁ to R₇ independently represent mono to a maximum possible number of substitutions, or no substitution; and
wherein each of R₁ to R₇ is independently selected from the group consisting of halogen, alkyl, phenyl, pyridyl, 3-biphenyl, 2-biphenyl, 4-biphenyl, 4-(3-pyridinyl)phenyl, 4-(2-pyridyl) phenyl, 4-(4-pyridyl)phenyl, 3-(3-pyridyl)phenyl, 3-(2-pyridyl) phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof.

2. An organic compound represented by one of the following formula (6) to formula (10), or tautomers thereof: 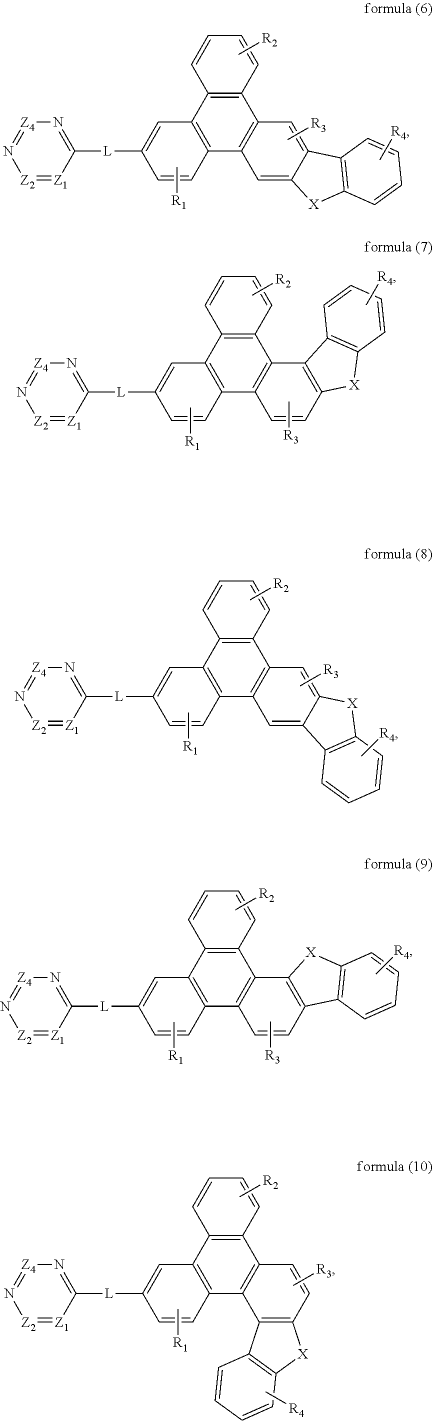
wherein X is selected from the group consisting of O, S, Se, NR₇ and SiR₅R₆;
where Z₁ is N or CR;
wherein Z₂ and Z₄ are CR;
wherein R is selected from the group consisting of H, phenyl, pyridyl, 3-biphenyl, 4-(pyridyl)phenyl, 3-(pyridyl)phenyl, 2-(pyridyl)phenyl, 9,9-dialkylfluorenyl, dibenzofuranyl, dibenzothienyl, and combinations thereof, wherein the S of the dibenzothienyl group in R is optionally changed to Se, and wherein the C connecting the dialkyl group in R is optionally changed to Si;
wherein L is a single bond, aryl or biphenyl;
wherein R₁ to R₇ independently represent mono to a maximum possible number of substitutions, or no substitution; and
wherein each of R₁ to R₇ is independently selected from the group consisting of halogen, alkyl, phenyl, pyridyl, 3-biphenyl, 2-biphenyl, 4-biphenyl, 4-(3-pyridinyl)phenyl, 4-(2-pyridyl) phenyl, 4-(4-pyridyl)phenyl, 3-(3-pyridyl)phenyl, 3-(2-pyridyl) phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, and combinations thereof.

3. The organic compound of claim 3, wherein X is selected from the group consisting of O, S Se and SiR₅R₆,
wherein R is selected from the group consisting of phenyl, 3-biphenyl, pyridylphenyl, 9,9-dialkylfluorenyl, dibenzofuranyl, dibenzothienyl, and combinations thereof;
wherein R₁ to R₆ independently represent mono to a maximum possible number of substitutions, or no substitution; and
wherein each of R₁ to R₆ is independently selected from the group consisting of methyl, phenyl, pyridyl, biphenyl, pyridylphenyl, 9,9-dialkylfluorenyl, dibenzofuranyl, dibenzothienyl, and combinations thereof.

4. The organic compound of claim 3, wherein one of the following is true:
only one of Rs is 3-biphenyl if X represents O;
R is phenyl if X represents CR₅R₆; and
X represents O or CR₅R₆ if R is phenyl, wherein R₅ and R₆ are methyl.

5. An organic compound selected from the group consisting of the following compounds C1 to C150:

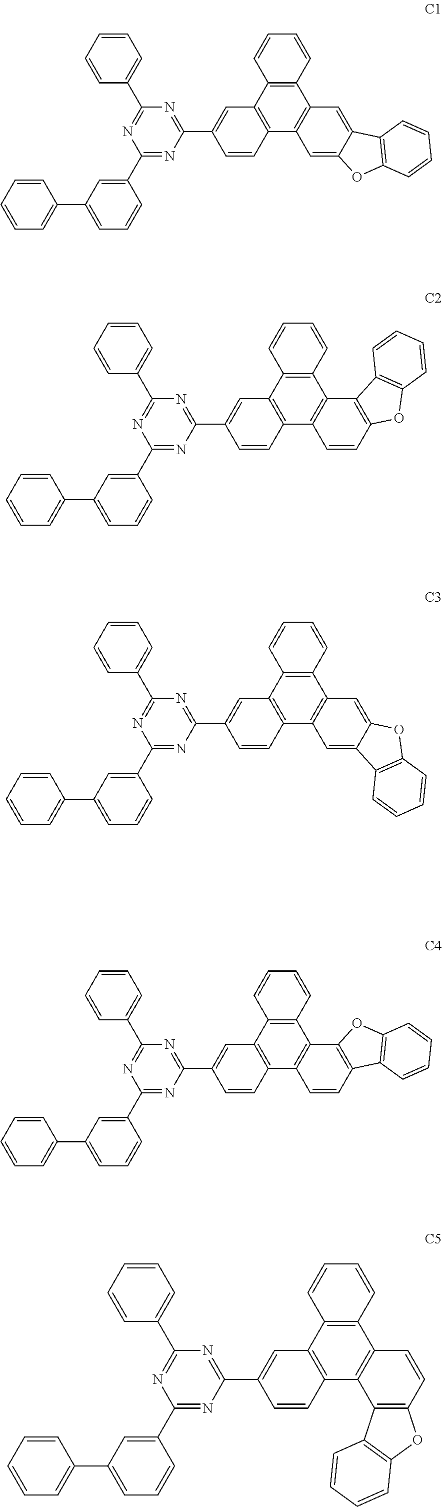 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; c1ccc(-c2cccc(-c3nc(-c4ccccc4)nc(-c4ccc5c(c4)c4ccccc4c4cc6oc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6oc54)n3)c2)cc1 ; c1ccc(-c2cccc(-c3nc(-c4ccccc4)nc(-c4ccc5c(c4)c4ccccc4c4ccc6oc7ccccc7c6c45)n3)c2)cc1]

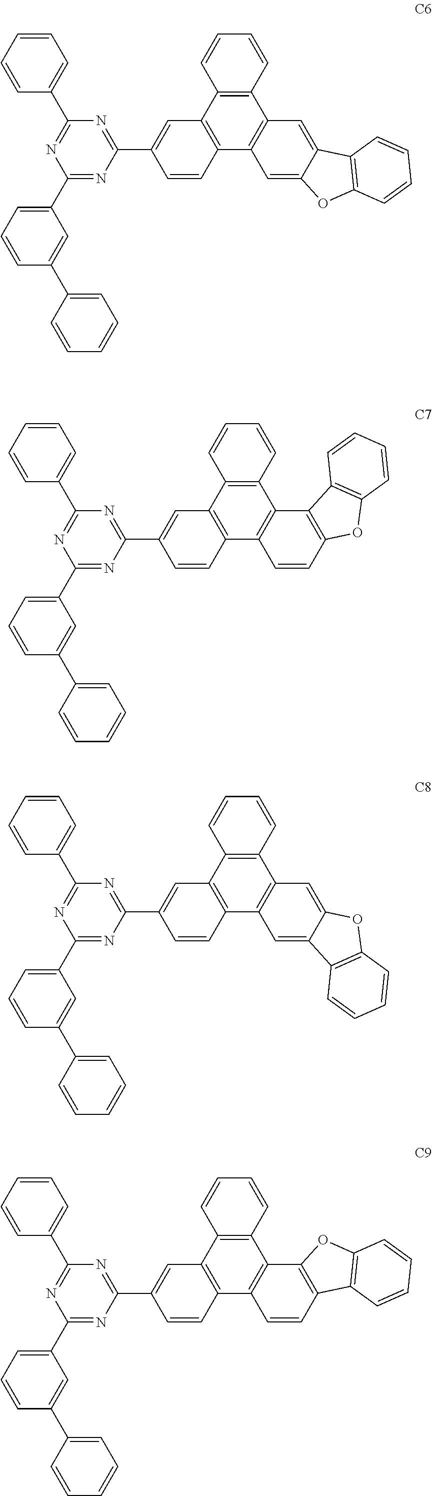 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; c1ccc(-c2cccc(-c3nc(-c4ccccc4)nc(-c4ccc5c(c4)c4ccccc4c4cc6oc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6oc54)n3)c2)cc1]

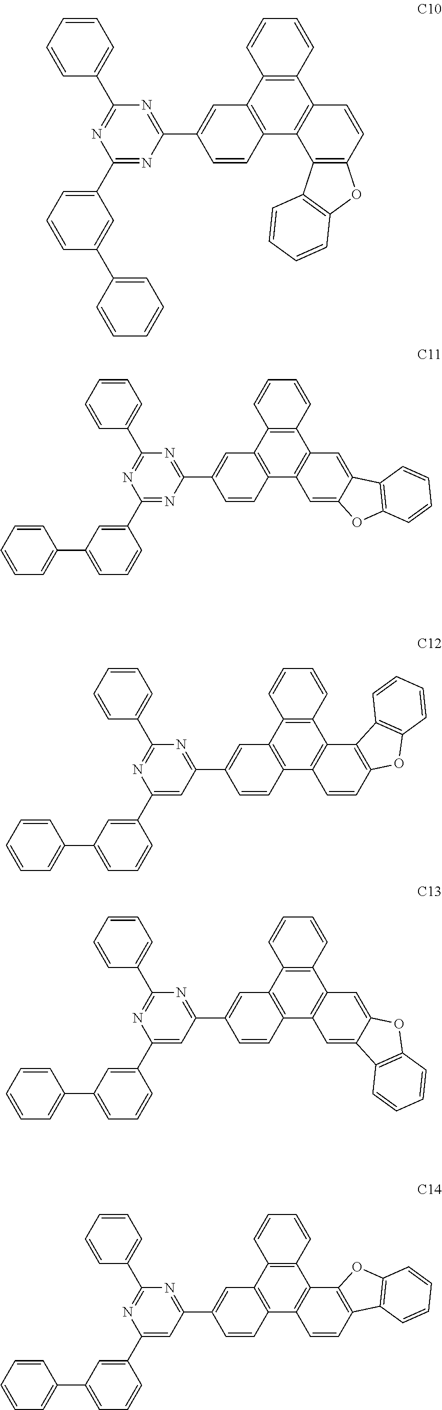 [SMILES: c1ccc(-c2cccc(-c3nc(-c4ccccc4)nc(-c4ccc5c(c4)c4ccccc4c4ccc6oc7ccccc7c6c45)n3)c2)cc1 ; c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1]

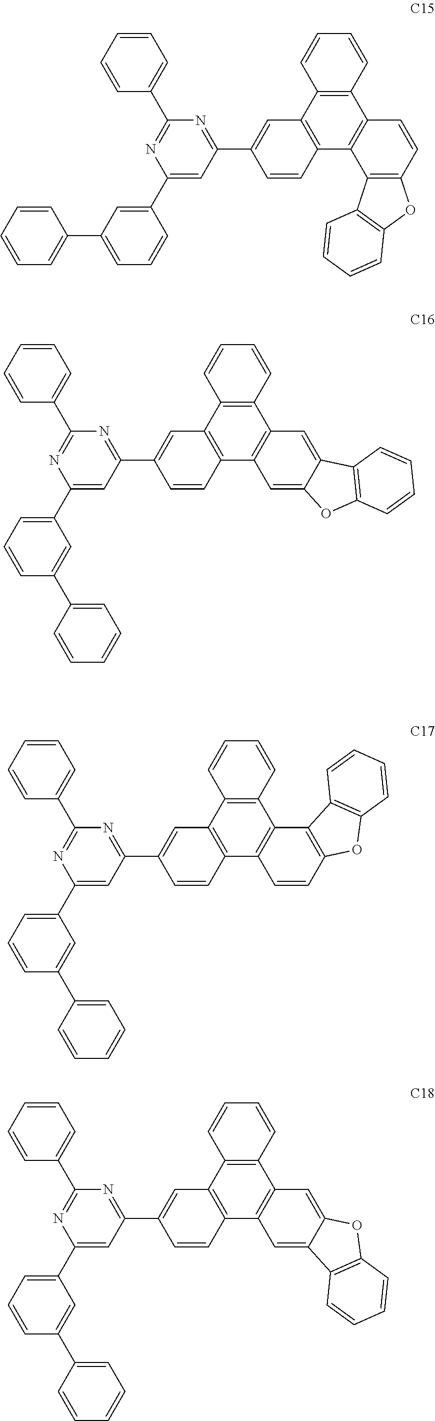 [SMILES: c1ccc(-c2cccc(-c3cc(-c4ccc5c(c4)c4ccccc4c4ccc6oc7ccccc7c6c45)nc(-c4ccccc4)n3)c2)cc1 ; c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4cc6oc7ccccc7c6cc54)nc(-c4ccccc4)n3)c2)cc1]

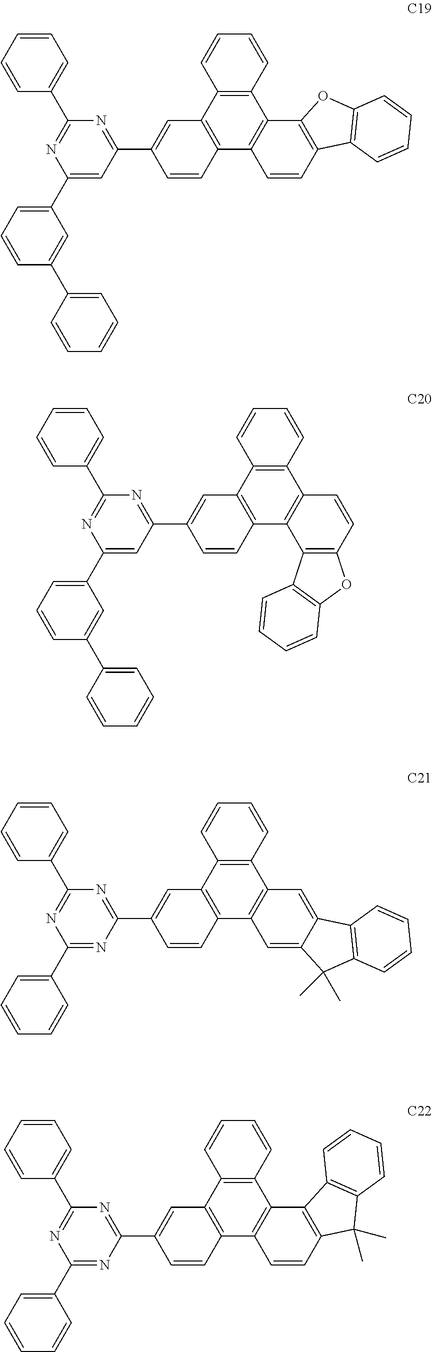 [SMILES: c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1 ; CC1(C)c2ccccc2-c2cc3c4ccccc4c4cc(-c5nc(-c6ccccc6)nc(-c6ccccc6)n5)ccc4c3cc21 ; CC1(C)c2ccccc2-c2c1ccc1c3ccc(-c4nc(-c5ccccc5)nc(-c5ccccc5)n4)cc3c3ccccc3c21]

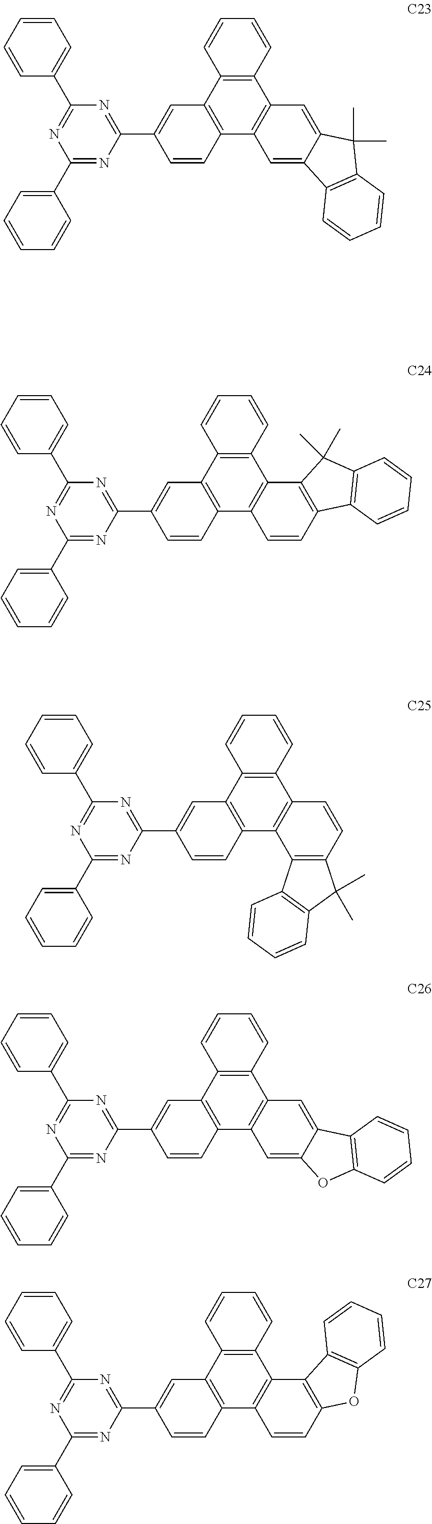 [SMILES: CC1(C)c2ccccc2-c2cc3c4ccc(-c5nc(-c6ccccc6)nc(-c6ccccc6)n5)cc4c4ccccc4c3cc21 ; CC1(C)c2ccccc2-c2ccc3c4ccc(-c5nc(-c6ccccc6)nc(-c6ccccc6)n5)cc4c4ccccc4c3c21 ; CC1(C)c2ccccc2-c2c1ccc1c3ccccc3c3cc(-c4nc(-c5ccccc5)nc(-c5ccccc5)n4)ccc3c21 ; c1ccc(-c2nc(-c3ccccc3)nc(-c3ccc4c(c3)c3ccccc3c3cc5c(cc43)oc3ccccc35)n2)cc1 ; c1ccc(-c2nc(-c3ccccc3)nc(-c3ccc4c(c3)c3ccccc3c3c4ccc4oc5ccccc5c43)n2)cc1]

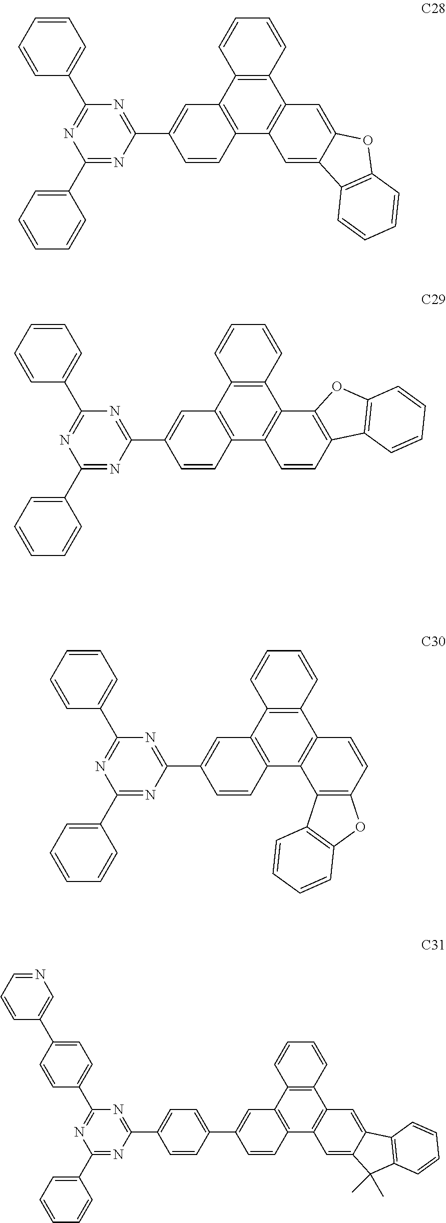 [SMILES: c1ccc(-c2nc(-c3ccccc3)nc(-c3ccc4c(c3)c3ccccc3c3cc5oc6ccccc6c5cc43)n2)cc1 ; c1ccc(-c2nc(-c3ccccc3)nc(-c3ccc4c(c3)c3ccccc3c3c4ccc4c5ccccc5oc43)n2)cc1 ; c1ccc(-c2nc(-c3ccccc3)nc(-c3ccc4c(c3)c3ccccc3c3ccc5oc6ccccc6c5c34)n2)cc1 ; CC1(C)c2ccccc2-c2cc3c4ccccc4c4cc(-c5ccc(-c6nc(-c7ccccc7)nc(-c7ccc(-c8cccnc8)cc7)n6)cc5)ccc4c3cc21]

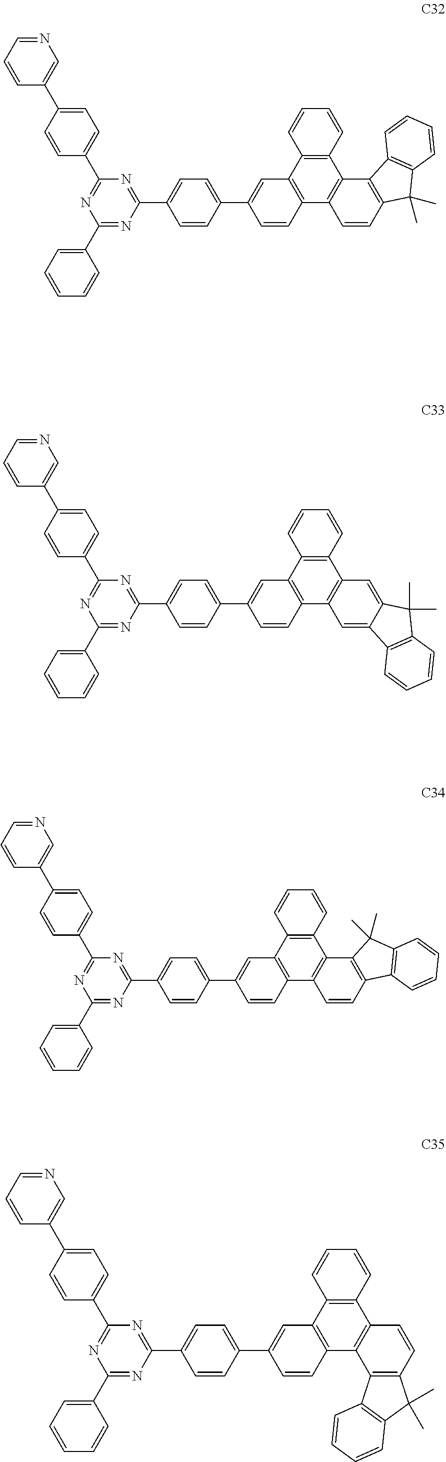 [SMILES: CC1(C)c2ccccc2-c2c1ccc1c3ccc(-c4ccc(-c5nc(-c6ccccc6)nc(-c6ccc(-c7cccnc7)cc6)n5)cc4)cc3c3ccccc3c21 ; CC1(C)c2ccccc2-c2cc3c4ccc(-c5ccc(-c6nc(-c7ccccc7)nc(-c7ccc(-c8cccnc8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8cccnc8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7cccnc7)cc6)n5)cc4)ccc3c21]

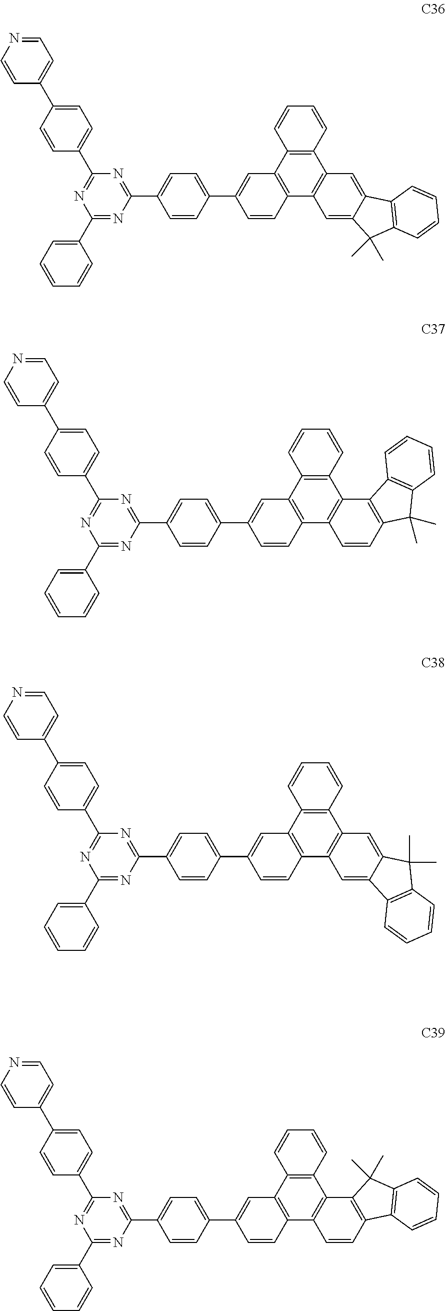 [SMILES: CC1(C)c2ccccc2-c2cc3c4ccccc4c4cc(-c5ccc(-c6nc(-c7ccccc7)nc(-c7ccc(-c8ccncc8)cc7)n6)cc5)ccc4c3cc21 ; CC1(C)c2ccccc2-c2c1ccc1c3ccc(-c4ccc(-c5nc(-c6ccccc6)nc(-c6ccc(-c7ccncc7)cc6)n5)cc4)cc3c3ccccc3c21 ; CC1(C)c2ccccc2-c2cc3c4ccc(-c5ccc(-c6nc(-c7ccccc7)nc(-c7ccc(-c8ccncc8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8ccncc8)cc7)n6)cc5)cc4c4ccccc4c3c21]

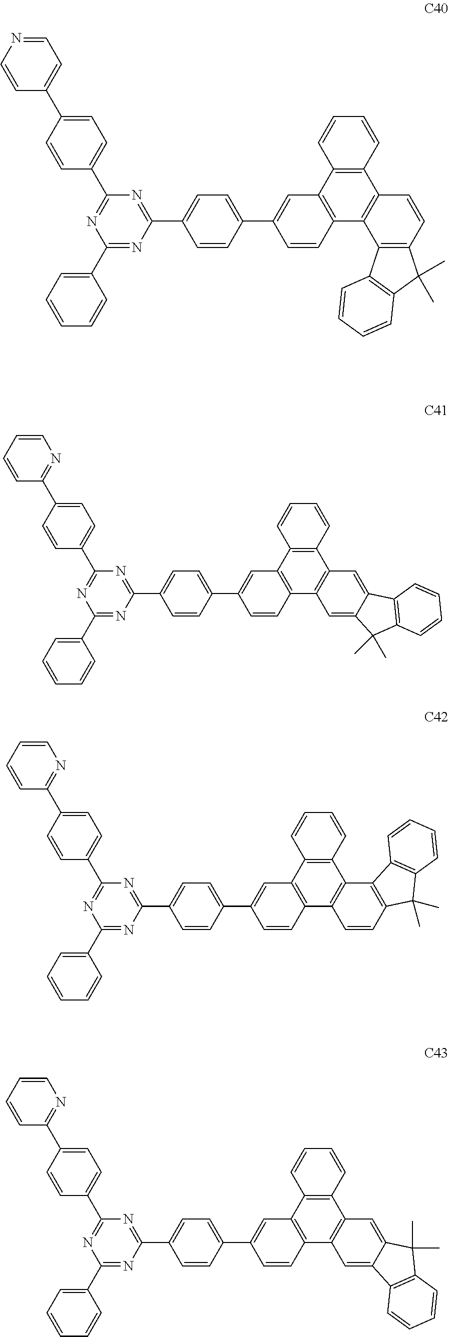 [SMILES: CC1(C)c2ccccc2-c2c1ccc1c3ccccc3c3cc(-c4ccc(-c5nc(-c6ccccc6)nc(-c6ccc(-c7ccncc7)cc6)n5)cc4)ccc3c21 ; CC1(C)c2ccccc2-c2cc3c4ccccc4c4cc(-c5ccc(-c6nc(-c7ccccc7)nc(-c7ccc(-c8ccccn8)cc7)n6)cc5)ccc4c3cc21 ; CC1(C)c2ccccc2-c2c1ccc1c3ccc(-c4ccc(-c5nc(-c6ccccc6)nc(-c6ccc(-c7ccccn7)cc6)n5)cc4)cc3c3ccccc3c21 ; CC1(C)c2ccccc2-c2cc3c4ccc(-c5ccc(-c6nc(-c7ccccc7)nc(-c7ccc(-c8ccccn8)cc7)n6)cc5)cc4c4ccccc4c3cc21]

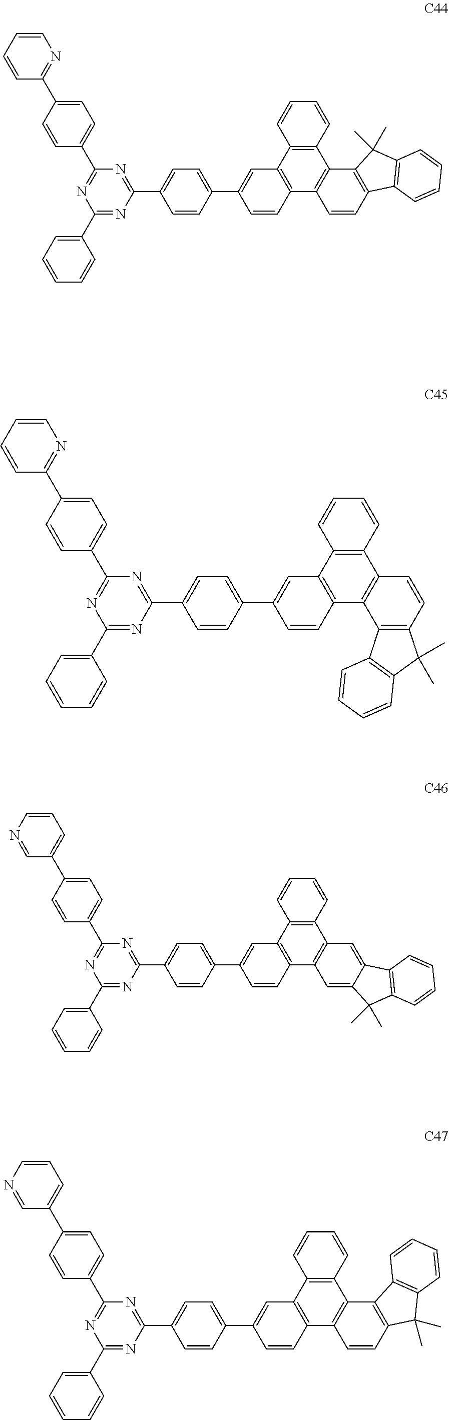 [SMILES: CC1(C)c2ccccc2-c2ccc3c4ccc(-c5ccc(-c6nc(-c7ccccc7)nc(-c7ccc(-c8ccccn8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7ccccn7)cc6)n5)cc4)ccc3c21 ; CC1(C)c2ccccc2-c2cc3c4ccccc4c4cc(-c5ccc(-c6nc(-c7ccccc7)nc(-c7ccc(-c8cccnc8)cc7)n6)cc5)ccc4c3cc21 ; CC1(C)c2ccccc2-c2c1ccc1c3ccc(-c4ccc(-c5nc(-c6ccccc6)nc(-c6ccc(-c7cccnc7)cc6)n5)cc4)cc3c3ccccc3c21]

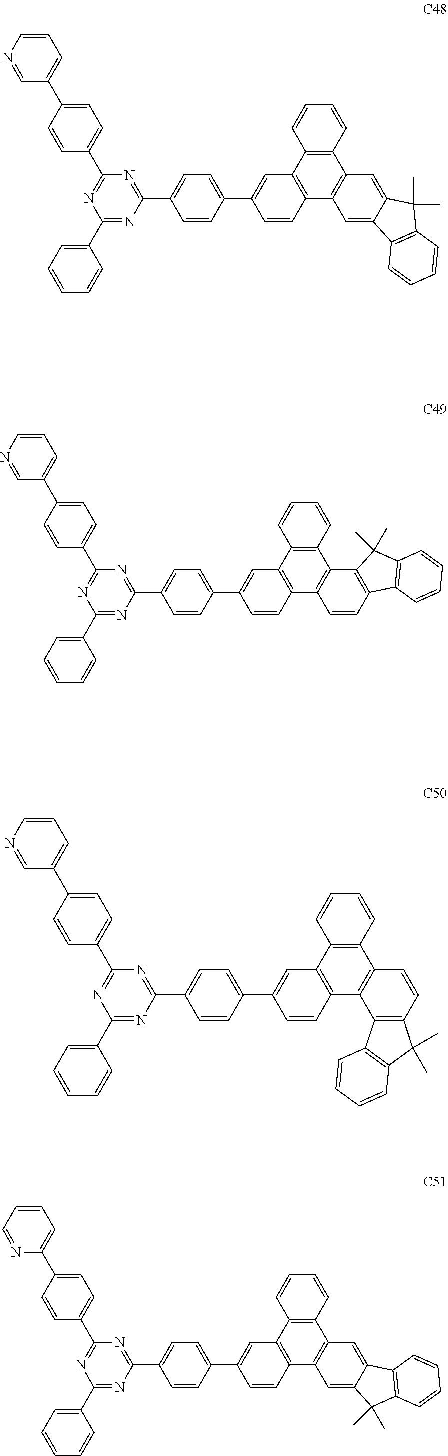 [SMILES: CC1(C)c2ccccc2-c2cc3c4ccc(-c5ccc(-c6nc(-c7ccccc7)nc(-c7ccc(-c8cccnc8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8cccnc8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7cccnc7)cc6)n5)cc4)ccc3c21 ; CC1(C)c2ccccc2-c2cc3c4ccccc4c4cc(-c5ccc(-c6nc(-c7ccccc7)nc(-c7ccc(-c8ccccn8)cc7)n6)cc5)ccc4c3cc21]

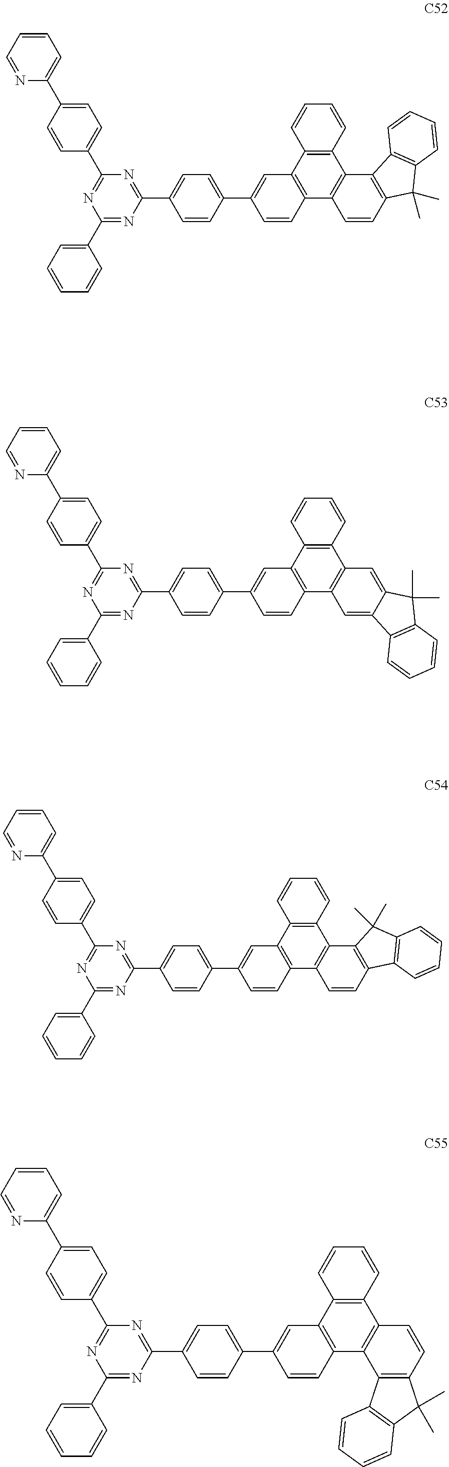 [SMILES: CC1(C)c2ccccc2-c2c1ccc1c3ccc(-c4ccc(-c5nc(-c6ccccc6)nc(-c6ccc(-c7ccccn7)cc6)n5)cc4)cc3c3ccccc3c21 ; CC1(C)c2ccccc2-c2cc3c4ccc(-c5ccc(-c6nc(-c7ccccc7)nc(-c7ccc(-c8ccccn8)cc7)n6)cc5)cc4c4ccccc4c3cc21 ; CC1(C)c2ccccc2-c2ccc3c4ccc(-c5ccc(-c6nc(-c7ccccc7)nc(-c7ccc(-c8ccccn8)cc7)n6)cc5)cc4c4ccccc4c3c21 ; CC1(C)c2ccccc2-c2c1ccc1c3ccccc3c3cc(-c4ccc(-c5nc(-c6ccccc6)nc(-c6ccc(-c7ccccn7)cc6)n5)cc4)ccc3c21]

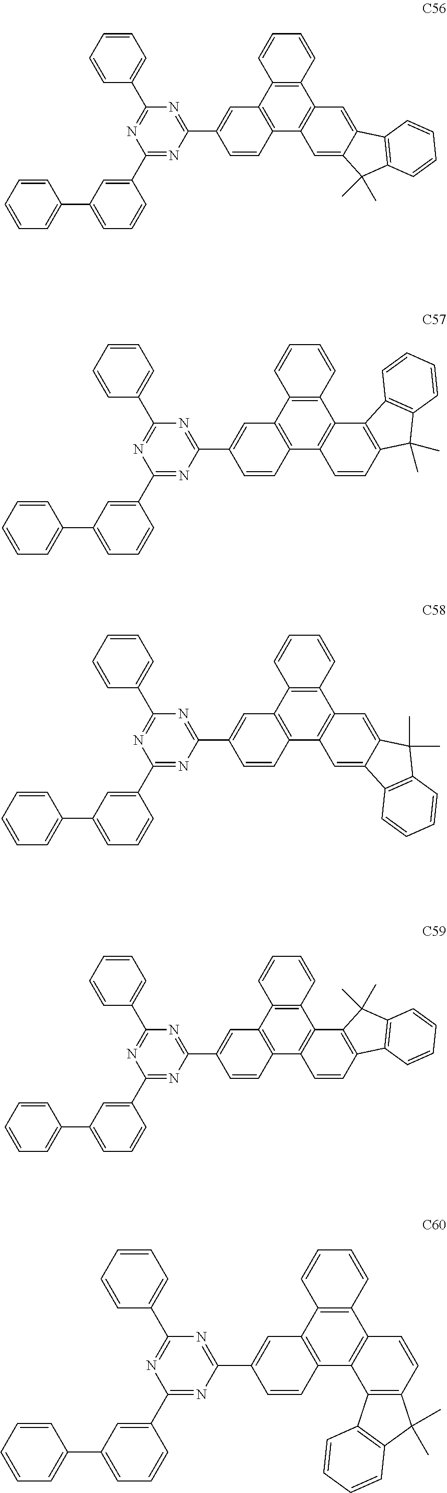 [SMILES: CC1(C)c2ccccc2-c2cc3c4ccccc4c4cc(-c5nc(-c6ccccc6)nc(-c6cccc(-c7ccccc7)c6)n5)ccc4c3cc21 ; CC1(C)c2ccccc2-c2c1ccc1c3ccc(-c4nc(-c5ccccc5)nc(-c5cccc(-c6ccccc6)c5)n4)cc3c3ccccc3c21 ; CC1(C)c2ccccc2-c2cc3c4ccc(-c5nc(-c6ccccc6)nc(-c6cccc(-c7ccccc7)c6)n5)cc4c4ccccc4c3cc21 ; CC1(C)c2ccccc2-c2ccc3c4ccc(-c5nc(-c6ccccc6)nc(-c6cccc(-c7ccccc7)c6)n5)cc4c4ccccc4c3c21 ; CC1(C)c2ccccc2-c2c1ccc1c3ccccc3c3cc(-c4nc(-c5ccccc5)nc(-c5cccc(-c6ccccc6)c5)n4)ccc3c21]

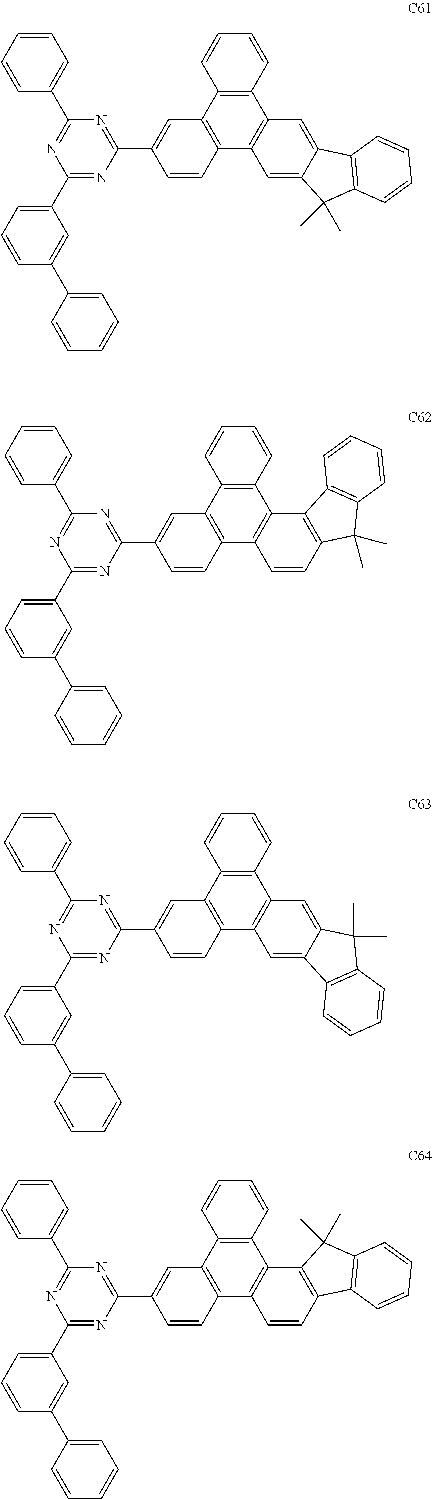 [SMILES: CC1(C)c2ccccc2-c2cc3c4ccccc4c4cc(-c5nc(-c6ccccc6)nc(-c6cccc(-c7ccccc7)c6)n5)ccc4c3cc21 ; CC1(C)c2ccccc2-c2c1ccc1c3ccc(-c4nc(-c5ccccc5)nc(-c5cccc(-c6ccccc6)c5)n4)cc3c3ccccc3c21 ; CC1(C)c2ccccc2-c2cc3c4ccc(-c5nc(-c6ccccc6)nc(-c6cccc(-c7ccccc7)c6)n5)cc4c4ccccc4c3cc21 ; CC1(C)c2ccccc2-c2ccc3c4ccc(-c5nc(-c6ccccc6)nc(-c6cccc(-c7ccccc7)c6)n5)cc4c4ccccc4c3c21]

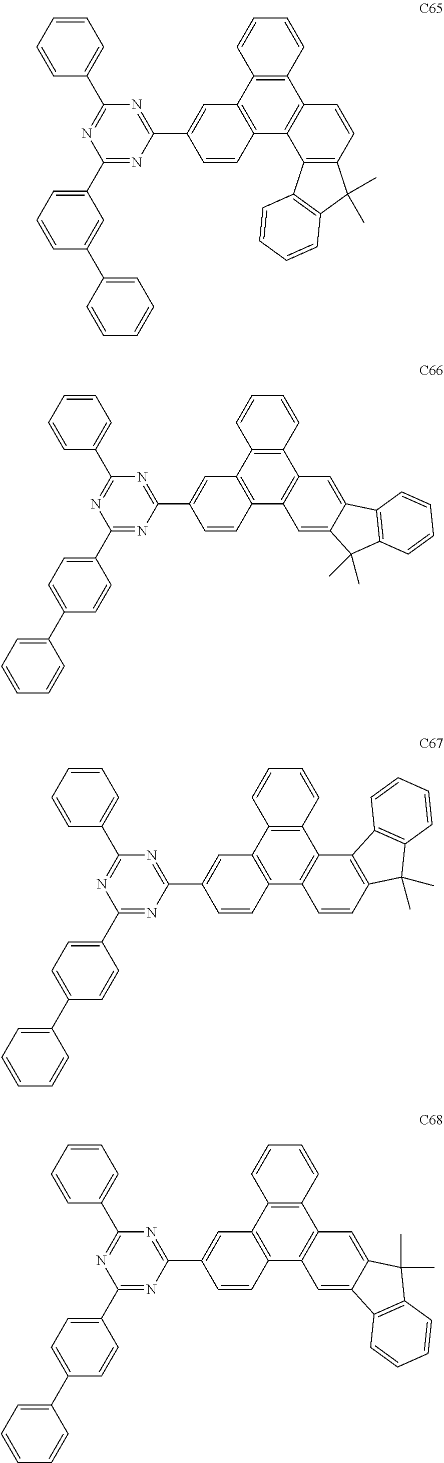 [SMILES: CC1(C)c2ccccc2-c2c1ccc1c3ccccc3c3cc(-c4nc(-c5ccccc5)nc(-c5cccc(-c6ccccc6)c5)n4)ccc3c21 ; CC1(C)c2ccccc2-c2cc3c4ccccc4c4cc(-c5nc(-c6ccccc6)nc(-c6ccc(-c7ccccc7)cc6)n5)ccc4c3cc21 ; CC1(C)c2ccccc2-c2c1ccc1c3ccc(-c4nc(-c5ccccc5)nc(-c5ccc(-c6ccccc6)cc5)n4)cc3c3ccccc3c21 ; CC1(C)c2ccccc2-c2cc3c4ccc(-c5nc(-c6ccccc6)nc(-c6ccc(-c7ccccc7)cc6)n5)cc4c4ccccc4c3cc21]

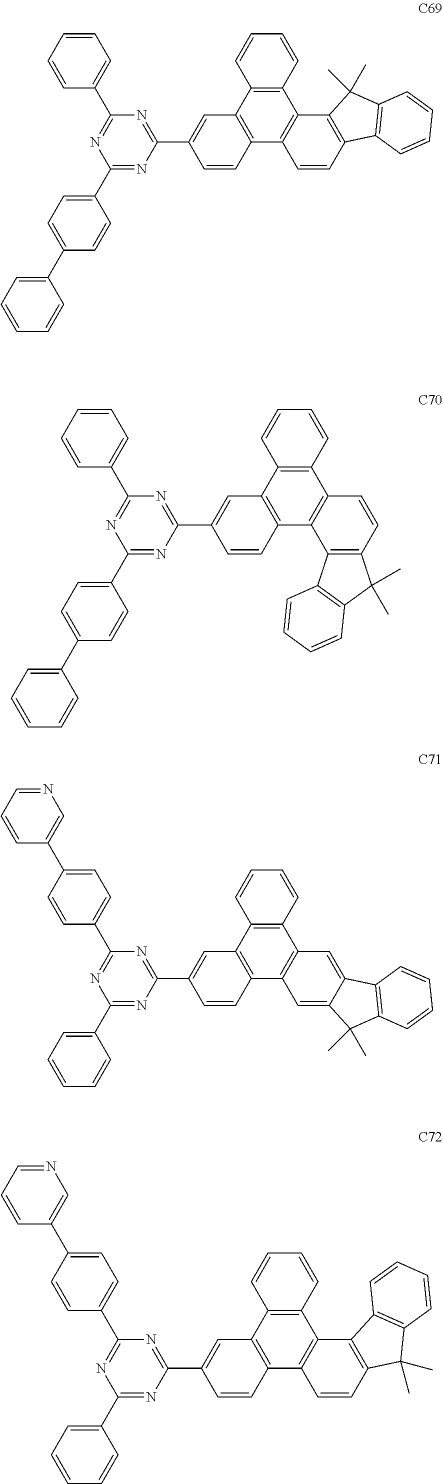 [SMILES: CC1(C)c2ccccc2-c2ccc3c4ccc(-c5nc(-c6ccccc6)nc(-c6ccc(-c7ccccc7)cc6)n5)cc4c4ccccc4c3c21 ; CC1(C)c2ccccc2-c2c1ccc1c3ccccc3c3cc(-c4nc(-c5ccccc5)nc(-c5ccc(-c6ccccc6)cc5)n4)ccc3c21 ; CC1(C)c2ccccc2-c2cc3c4ccccc4c4cc(-c5nc(-c6ccccc6)nc(-c6ccc(-c7cccnc7)cc6)n5)ccc4c3cc21 ; CC1(C)c2ccccc2-c2c1ccc1c3ccc(-c4nc(-c5ccccc5)nc(-c5ccc(-c6cccnc6)cc5)n4)cc3c3ccccc3c21]

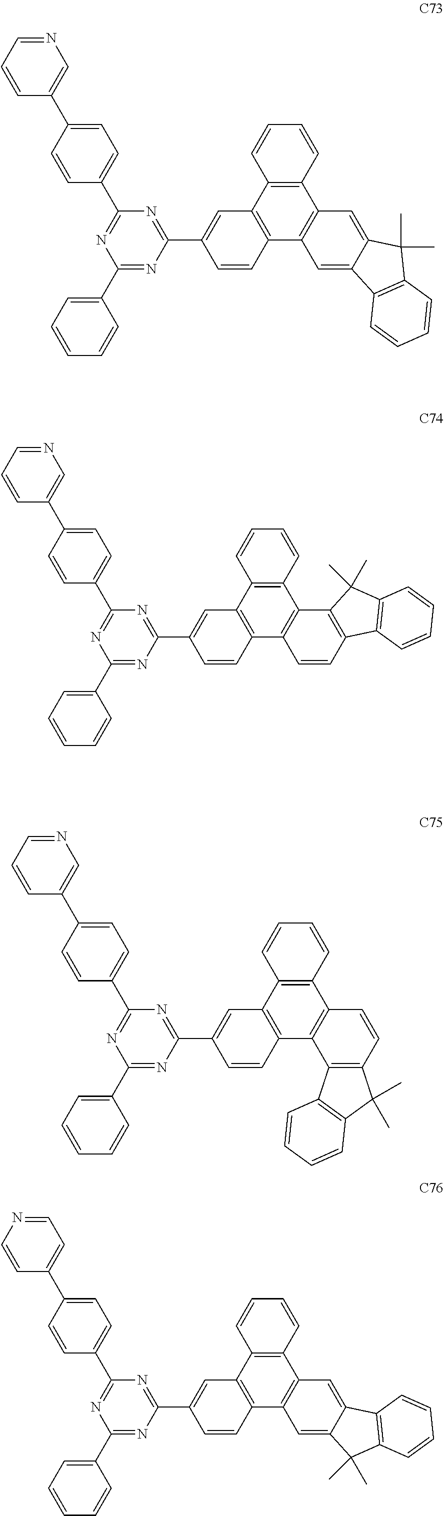 [SMILES: CC1(C)c2ccccc2-c2cc3c4ccc(-c5nc(-c6ccccc6)nc(-c6ccc(-c7cccnc7)cc6)n5)cc4c4ccccc4c3cc21 ; CC1(C)c2ccccc2-c2ccc3c4ccc(-c5nc(-c6ccccc6)nc(-c6ccc(-c7cccnc7)cc6)n5)cc4c4ccccc4c3c21 ; CC1(C)c2ccccc2-c2c1ccc1c3ccccc3c3cc(-c4nc(-c5ccccc5)nc(-c5ccc(-c6cccnc6)cc5)n4)ccc3c21 ; CC1(C)c2ccccc2-c2cc3c4ccccc4c4cc(-c5nc(-c6ccccc6)nc(-c6ccc(-c7ccncc7)cc6)n5)ccc4c3cc21]

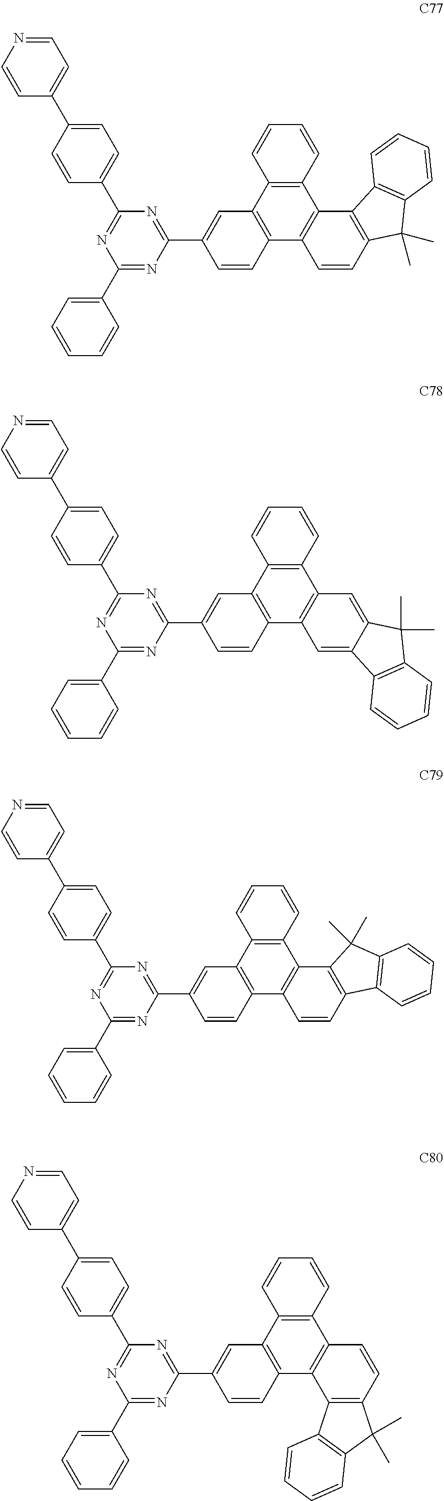 [SMILES: CC1(C)c2ccccc2-c2c1ccc1c3ccc(-c4nc(-c5ccccc5)nc(-c5ccc(-c6ccncc6)cc5)n4)cc3c3ccccc3c21 ; CC1(C)c2ccccc2-c2cc3c4ccc(-c5nc(-c6ccccc6)nc(-c6ccc(-c7ccncc7)cc6)n5)cc4c4ccccc4c3cc21 ; CC1(C)c2ccccc2-c2ccc3c4ccc(-c5nc(-c6ccccc6)nc(-c6ccc(-c7ccncc7)cc6)n5)cc4c4ccccc4c3c21 ; CC1(C)c2ccccc2-c2c1ccc1c3ccccc3c3cc(-c4nc(-c5ccccc5)nc(-c5ccc(-c6ccncc6)cc5)n4)ccc3c21]

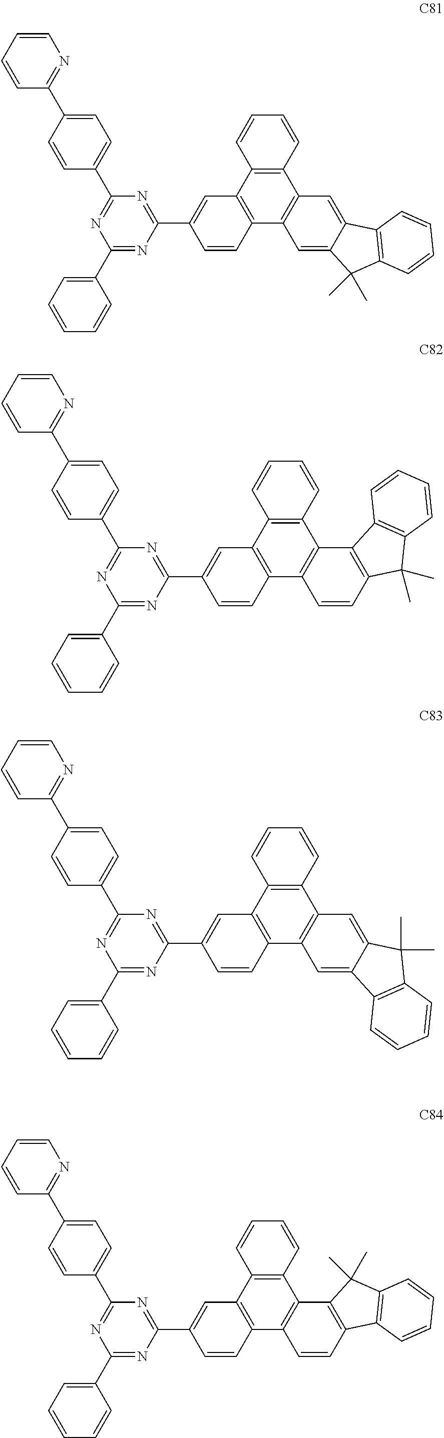

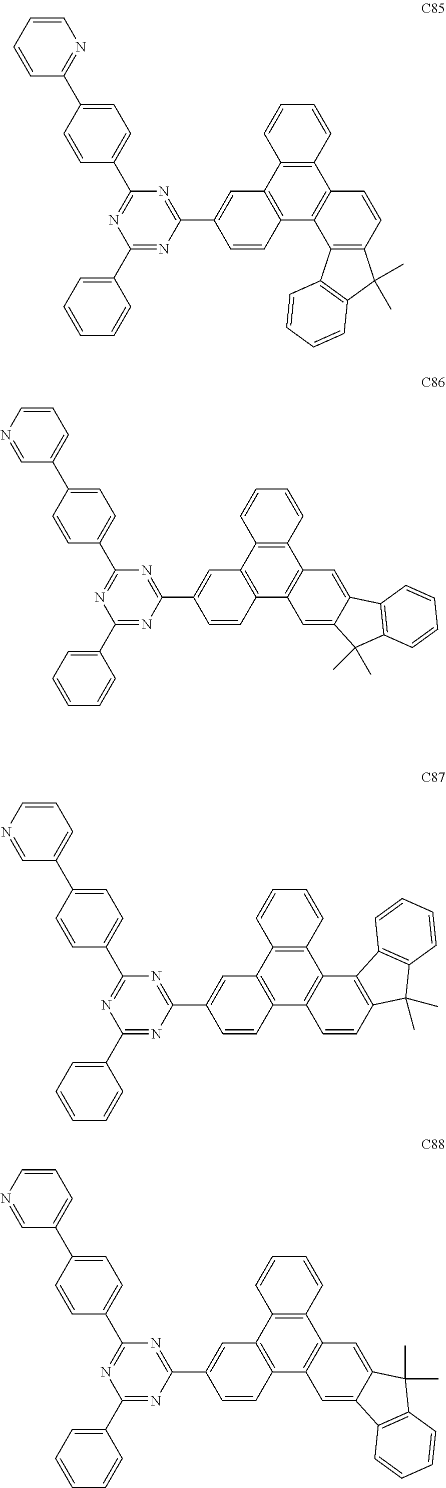 [SMILES: CC1(C)c2ccccc2-c2c1ccc1c3ccccc3c3cc(-c4nc(-c5ccccc5)nc(-c5ccc(-c6ccccn6)cc5)n4)ccc3c21 ; CC1(C)c2ccccc2-c2cc3c4ccccc4c4cc(-c5nc(-c6ccccc6)nc(-c6ccc(-c7cccnc7)cc6)n5)ccc4c3cc21 ; CC1(C)c2ccccc2-c2c1ccc1c3ccc(-c4nc(-c5ccccc5)nc(-c5ccc(-c6cccnc6)cc5)n4)cc3c3ccccc3c21 ; CC1(C)c2ccccc2-c2cc3c4ccc(-c5nc(-c6ccccc6)nc(-c6ccc(-c7cccnc7)cc6)n5)cc4c4ccccc4c3cc21]

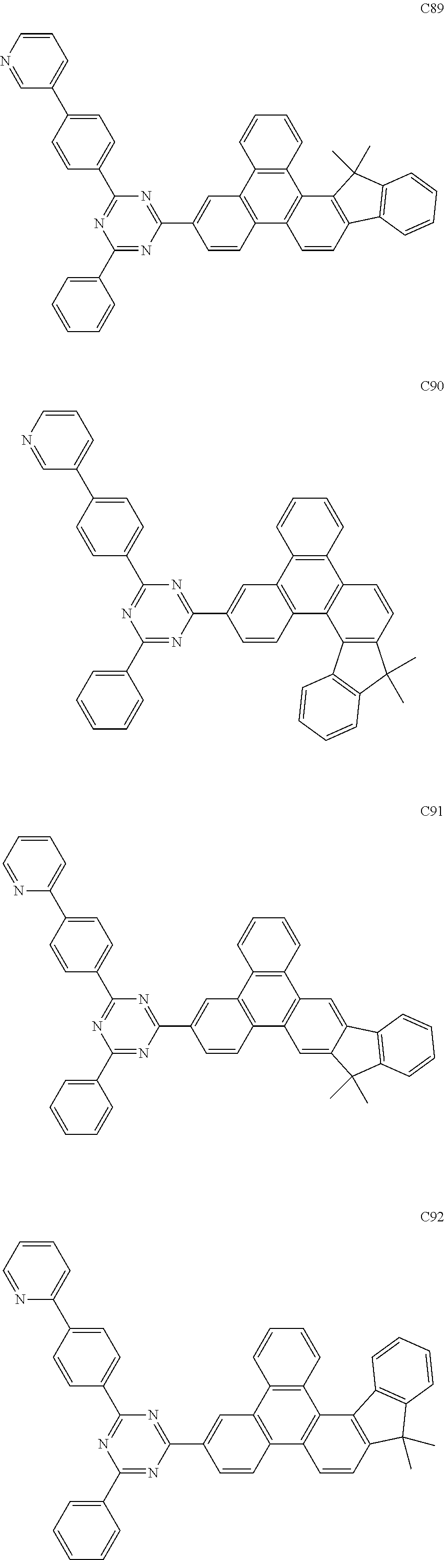 [SMILES: CC1(C)c2ccccc2-c2ccc3c4ccc(-c5nc(-c6ccccc6)nc(-c6ccc(-c7cccnc7)cc6)n5)cc4c4ccccc4c3c21 ; CC1(C)c2ccccc2-c2c1ccc1c3ccccc3c3cc(-c4nc(-c5ccccc5)nc(-c5ccc(-c6cccnc6)cc5)n4)ccc3c21 ; CC1(C)c2ccccc2-c2cc3c4ccccc4c4cc(-c5nc(-c6ccccc6)nc(-c6ccc(-c7ccccn7)cc6)n5)ccc4c3cc21 ; CC1(C)c2ccccc2-c2c1ccc1c3ccc(-c4nc(-c5ccccc5)nc(-c5ccc(-c6ccccn6)cc5)n4)cc3c3ccccc3c21]

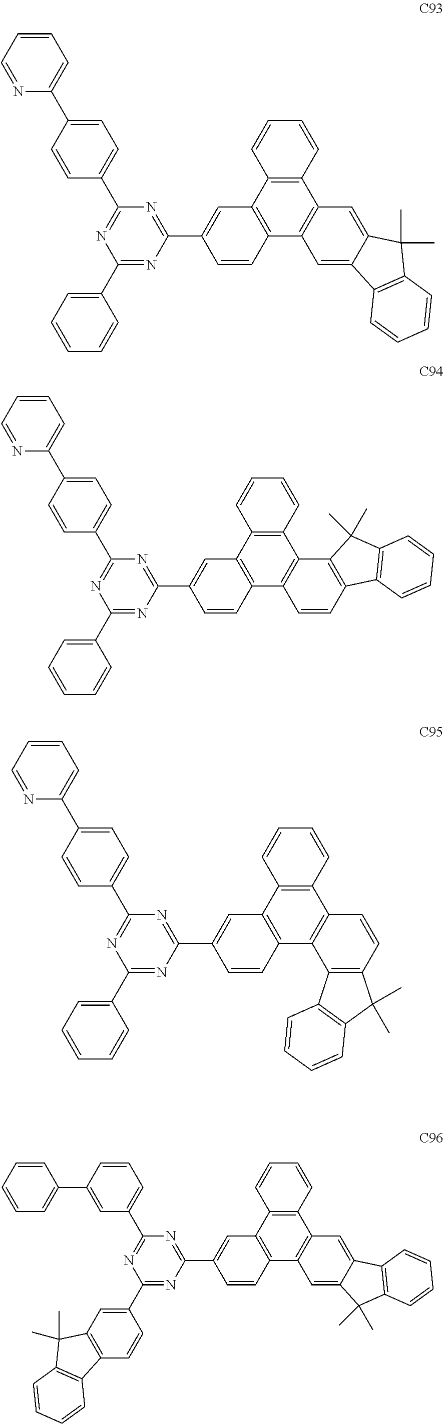 [SMILES: CC1(C)c2ccccc2-c2cc3c4ccc(-c5nc(-c6ccccc6)nc(-c6ccc(-c7ccccn7)cc6)n5)cc4c4ccccc4c3cc21 ; CC1(C)c2ccccc2-c2ccc3c4ccc(-c5nc(-c6ccccc6)nc(-c6ccc(-c7ccccn7)cc6)n5)cc4c4ccccc4c3c21 ; CC1(C)c2ccccc2-c2c1ccc1c3ccccc3c3cc(-c4nc(-c5ccccc5)nc(-c5ccc(-c6ccccn6)cc5)n4)ccc3c21 ; CC1(C)c2ccccc2-c2ccc(-c3nc(-c4cccc(-c5ccccc5)c4)nc(-c4ccc5c(c4)c4ccccc4c4cc6c(cc54)C(C)(C)c4ccccc4-6)n3)cc21]

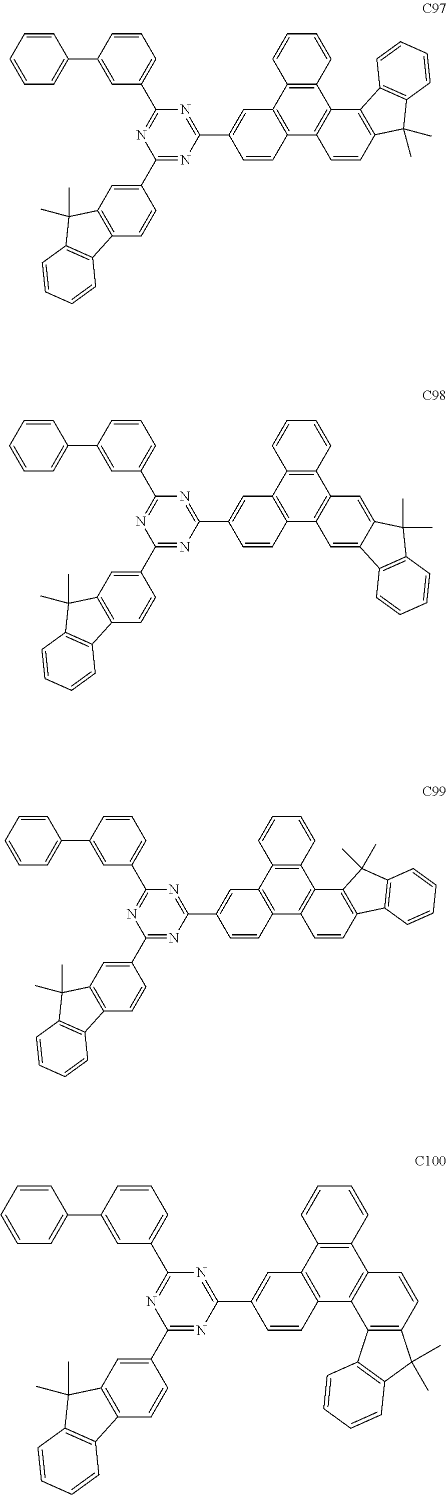 [SMILES: CC1(C)c2ccccc2-c2ccc(-c3nc(-c4cccc(-c5ccccc5)c4)nc(-c4ccc5c(c4)c4ccccc4c4c6c(ccc54)C(C)(C)c4ccccc4-6)n3)cc21 ; CC1(C)c2ccccc2-c2ccc(-c3nc(-c4cccc(-c5ccccc5)c4)nc(-c4ccc5c(c4)c4ccccc4c4cc6c(cc54)-c4ccccc4C6(C)C)n3)cc21 ; CC1(C)c2ccccc2-c2ccc(-c3nc(-c4cccc(-c5ccccc5)c4)nc(-c4ccc5c(c4)c4ccccc4c4c6c(ccc54)-c4ccccc4C6(C)C)n3)cc21 ; CC1(C)c2ccccc2-c2ccc(-c3nc(-c4cccc(-c5ccccc5)c4)nc(-c4ccc5c(c4)c4ccccc4c4ccc6c(c45)-c4ccccc4C6(C)C)n3)cc21]

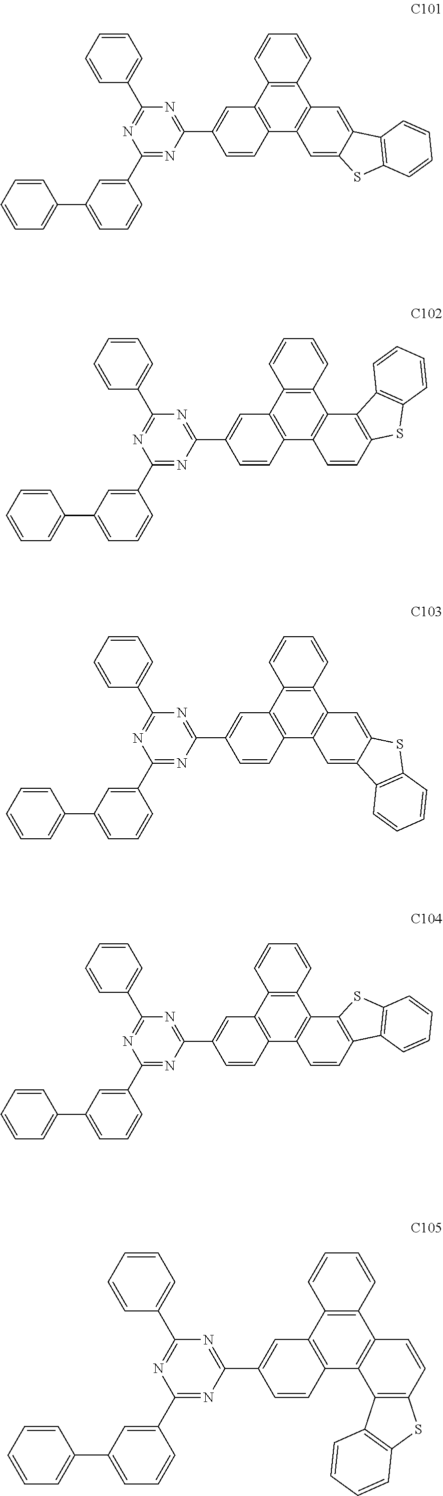 [SMILES: c1ccc(-c2cccc(-c3nc(-c4ccccc4)nc(-c4ccc5c(c4)c4ccccc4c4cc6c(cc54)sc4ccccc46)n3)c2)cc1 ; c1ccc(-c2cccc(-c3nc(-c4ccccc4)nc(-c4ccc5c(c4)c4ccccc4c4c5ccc5sc6ccccc6c54)n3)c2)cc1 ; c1ccc(-c2cccc(-c3nc(-c4ccccc4)nc(-c4ccc5c(c4)c4ccccc4c4cc6sc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6sc54)n3)c2)cc1 ; c1ccc(-c2cccc(-c3nc(-c4ccccc4)nc(-c4ccc5c(c4)c4ccccc4c4ccc6sc7ccccc7c6c45)n3)c2)cc1]

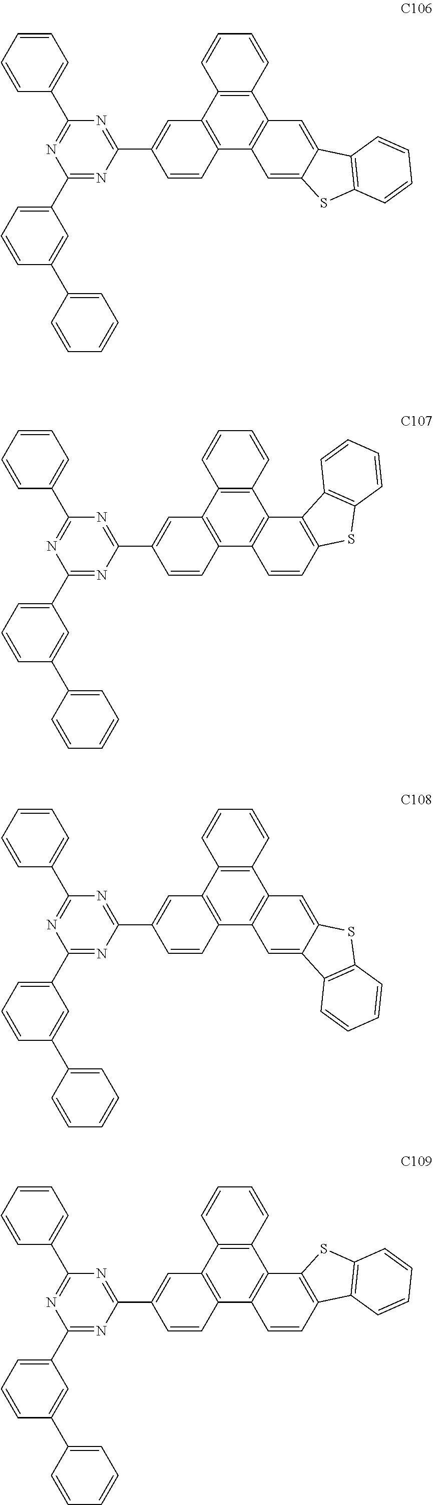 [SMILES: c1ccc(-c2cccc(-c3nc(-c4ccccc4)nc(-c4ccc5c(c4)c4ccccc4c4cc6c(cc54)sc4ccccc46)n3)c2)cc1 ; c1ccc(-c2cccc(-c3nc(-c4ccccc4)nc(-c4ccc5c(c4)c4ccccc4c4c5ccc5sc6ccccc6c54)n3)c2)cc1 ; c1ccc(-c2cccc(-c3nc(-c4ccccc4)nc(-c4ccc5c(c4)c4ccccc4c4cc6sc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6sc54)n3)c2)cc1]

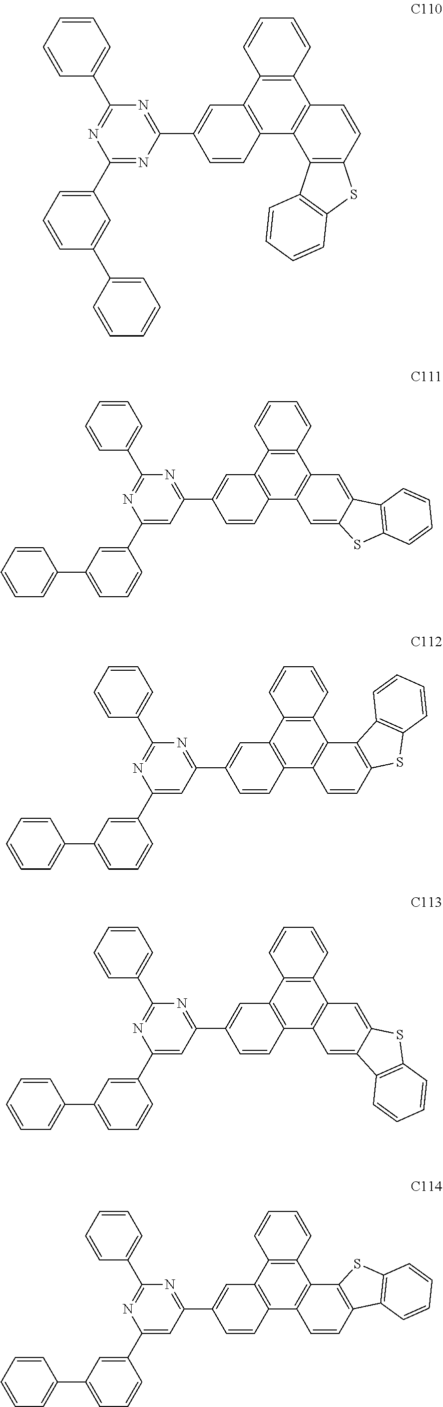 [SMILES: c1ccc(-c2cccc(-c3nc(-c4ccccc4)nc(-c4ccc5c(c4)c4ccccc4c4ccc6sc7ccccc7c6c45)n3)c2)cc1 ; c1ccc(-c2cccc(-c3cc(-c4ccc5c(c4)c4ccccc4c4cc6c(cc54)sc4ccccc46)nc(-c4ccccc4)n3)c2)cc1 ; c1ccc(-c2cccc(-c3cc(-c4ccc5c(c4)c4ccccc4c4c5ccc5sc6ccccc6c54)nc(-c4ccccc4)n3)c2)cc1 ; c1ccc(-c2cccc(-c3cc(-c4ccc5c(c4)c4ccccc4c4cc6sc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6sc54)nc(-c4ccccc4)n3)c2)cc1]

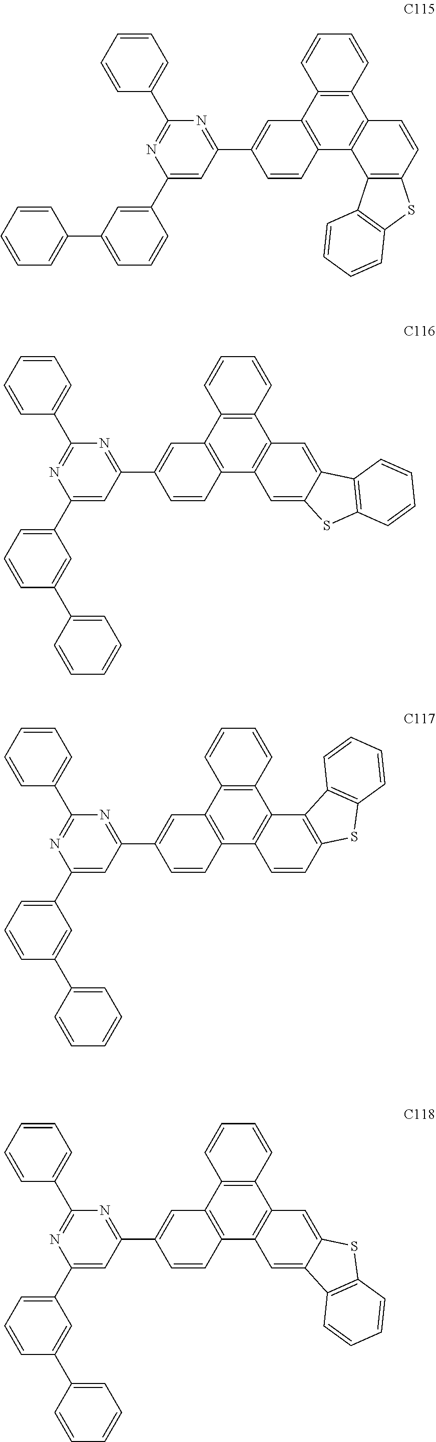 [SMILES: c1ccc(-c2cccc(-c3cc(-c4ccc5c(c4)c4ccccc4c4ccc6sc7ccccc7c6c45)nc(-c4ccccc4)n3)c2)cc1 ; c1ccc(-c2cccc(-c3cc(-c4ccc5c(c4)c4ccccc4c4cc6c(cc54)sc4ccccc46)nc(-c4ccccc4)n3)c2)cc1 ; c1ccc(-c2cccc(-c3cc(-c4ccc5c(c4)c4ccccc4c4c5ccc5sc6ccccc6c54)nc(-c4ccccc4)n3)c2)cc1 ; c1ccc(-c2cccc(-c3cc(-c4ccc5c(c4)c4ccccc4c4cc6sc7ccccc7c6cc54)nc(-c4ccccc4)n3)c2)cc1]

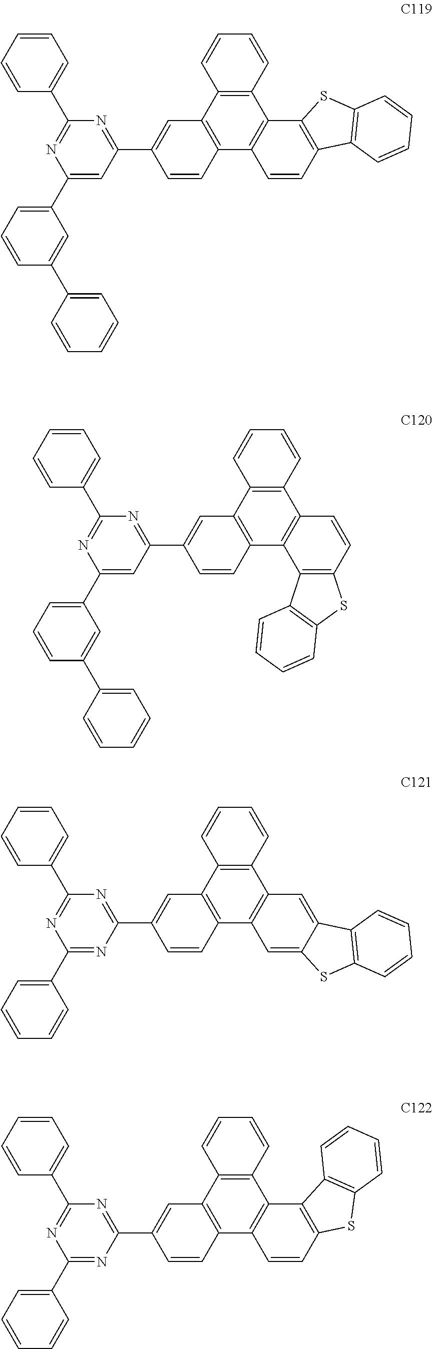 [SMILES: c1ccc(-c2cccc(-c3cc(-c4ccc5c(c4)c4ccccc4c4c5ccc5c6ccccc6sc54)nc(-c4ccccc4)n3)c2)cc1 ; c1ccc(-c2cccc(-c3cc(-c4ccc5c(c4)c4ccccc4c4ccc6sc7ccccc7c6c45)nc(-c4ccccc4)n3)c2)cc1 ; c1ccc(-c2nc(-c3ccccc3)nc(-c3ccc4c(c3)c3ccccc3c3cc5c(cc43)sc3ccccc35)n2)cc1 ; c1ccc(-c2nc(-c3ccccc3)nc(-c3ccc4c(c3)c3ccccc3c3c4ccc4sc5ccccc5c43)n2)cc1]

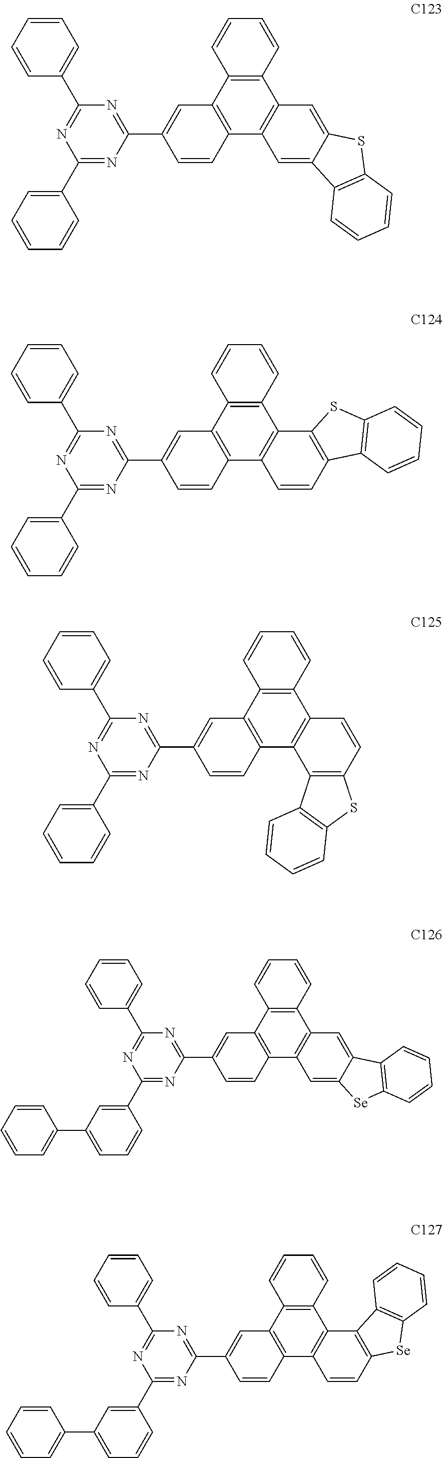 [SMILES: c1ccc(-c2nc(-c3ccccc3)nc(-c3ccc4c(c3)c3ccccc3c3cc5sc6ccccc6c5cc43)n2)cc1 ; c1ccc(-c2nc(-c3ccccc3)nc(-c3ccc4c(c3)c3ccccc3c3c4ccc4c5ccccc5sc43)n2)cc1 ; c1ccc(-c2nc(-c3ccccc3)nc(-c3ccc4c(c3)c3ccccc3c3ccc5sc6ccccc6c5c34)n2)cc1 ; c1ccc(-c2cccc(-c3nc(-c4ccccc4)nc(-c4ccc5c(c4)c4ccccc4c4cc6c(cc54)[se]c4ccccc46)n3)c2)cc1 ; c1ccc(-c2cccc(-c3nc(-c4ccccc4)nc(-c4ccc5c(c4)c4ccccc4c4c5ccc5[se]c6ccccc6c54)n3)c2)cc1]

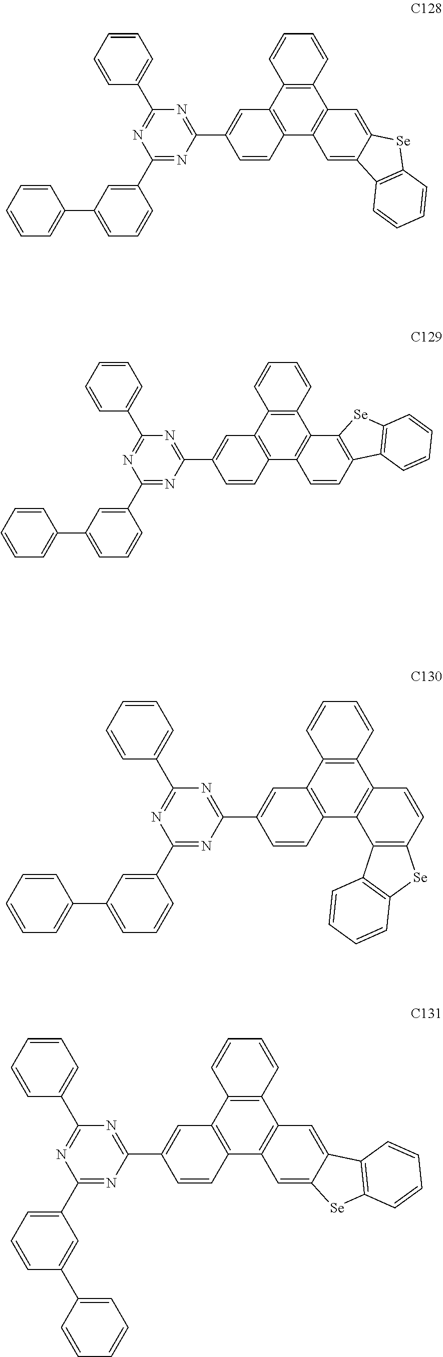 [SMILES: c1ccc(-c2cccc(-c3nc(-c4ccccc4)nc(-c4ccc5c(c4)c4ccccc4c4cc6[se]c7ccccc7c6cc54)n3)c2)cc1 ; c1ccc(-c2cccc(-c3nc(-c4ccccc4)nc(-c4ccc5c(c4)c4ccccc4c4c5ccc5c6ccccc6[se]c54)n3)c2)cc1 ; c1ccc(-c2cccc(-c3nc(-c4ccccc4)nc(-c4ccc5c(c4)c4ccccc4c4ccc6[se]c7ccccc7c6c45)n3)c2)cc1 ; c1ccc(-c2cccc(-c3nc(-c4ccccc4)nc(-c4ccc5c(c4)c4ccccc4c4cc6c(cc54)[se]c4ccccc46)n3)c2)cc1]

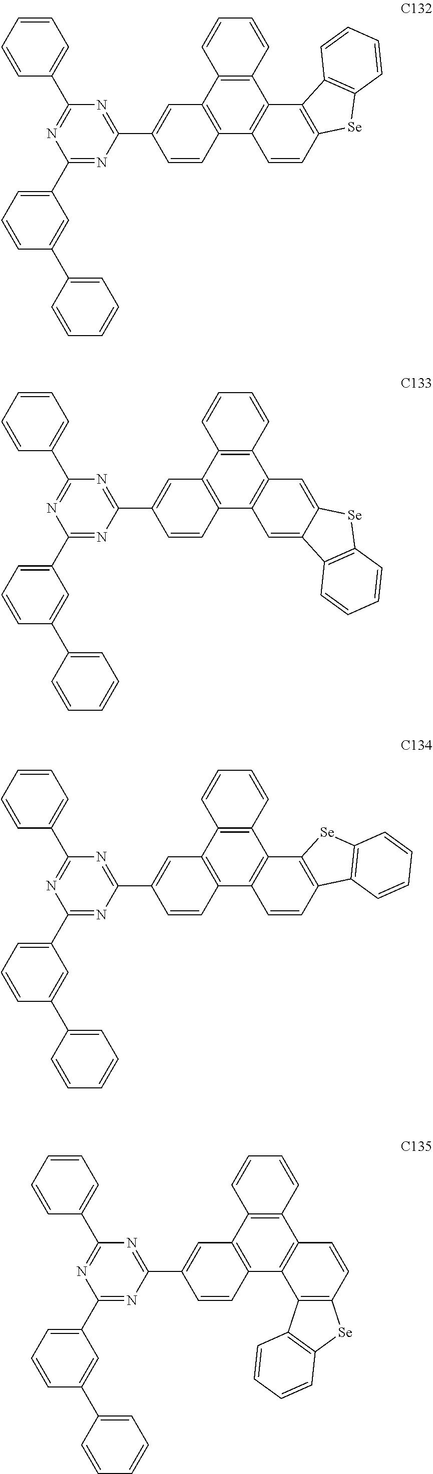 [SMILES: c1ccc(-c2cccc(-c3nc(-c4ccccc4)nc(-c4ccc5c(c4)c4ccccc4c4c5ccc5[se]c6ccccc6c54)n3)c2)cc1 ; c1ccc(-c2cccc(-c3nc(-c4ccccc4)nc(-c4ccc5c(c4)c4ccccc4c4cc6[se]c7ccccc7c6cc54)n3)c2)cc1 ; c1ccc(-c2cccc(-c3nc(-c4ccccc4)nc(-c4ccc5c(c4)c4ccccc4c4c5ccc5c6ccccc6[se]c54)n3)c2)cc1 ; c1ccc(-c2cccc(-c3nc(-c4ccccc4)nc(-c4ccc5c(c4)c4ccccc4c4ccc6[se]c7ccccc7c6c45)n3)c2)cc1]

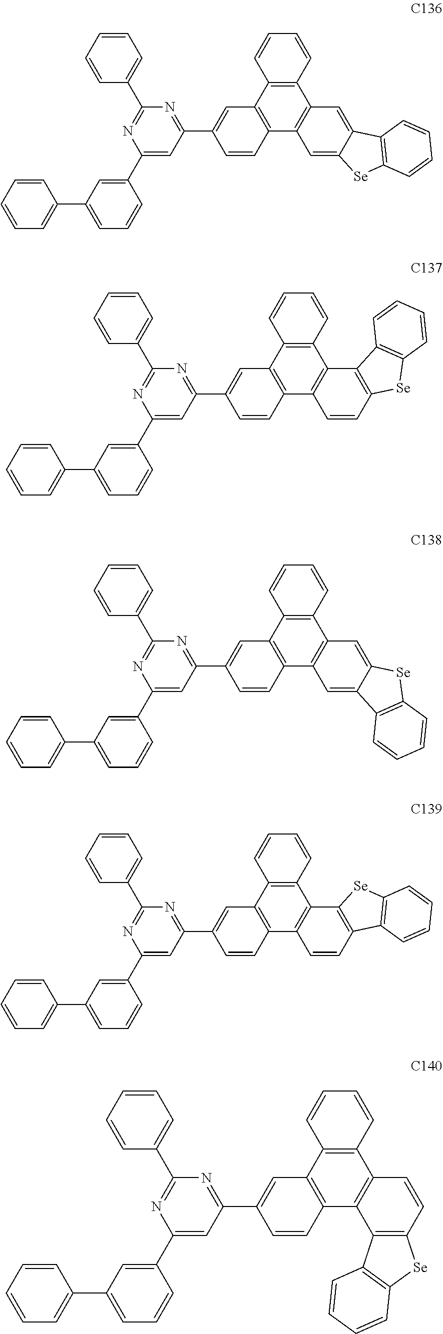 [SMILES: c1ccc(-c2cccc(-c3cc(-c4ccc5c(c4)c4ccccc4c4cc6c(cc54)[se]c4ccccc46)nc(-c4ccccc4)n3)c2)cc1 ; c1ccc(-c2cccc(-c3cc(-c4ccc5c(c4)c4ccccc4c4c5ccc5[se]c6ccccc6c54)nc(-c4ccccc4)n3)c2)cc1 ; c1ccc(-c2cccc(-c3cc(-c4ccc5c(c4)c4ccccc4c4cc6[se]c7ccccc7c6cc54)nc(-c4ccccc4)n3)c2)cc1 ; c1ccc(-c2cccc(-c3cc(-c4ccc5c(c4)c4ccccc4c4c5ccc5c6ccccc6[se]c54)nc(-c4ccccc4)n3)c2)cc1 ; c1ccc(-c2cccc(-c3cc(-c4ccc5c(c4)c4ccccc4c4ccc6[se]c7ccccc7c6c45)nc(-c4ccccc4)n3)c2)cc1]

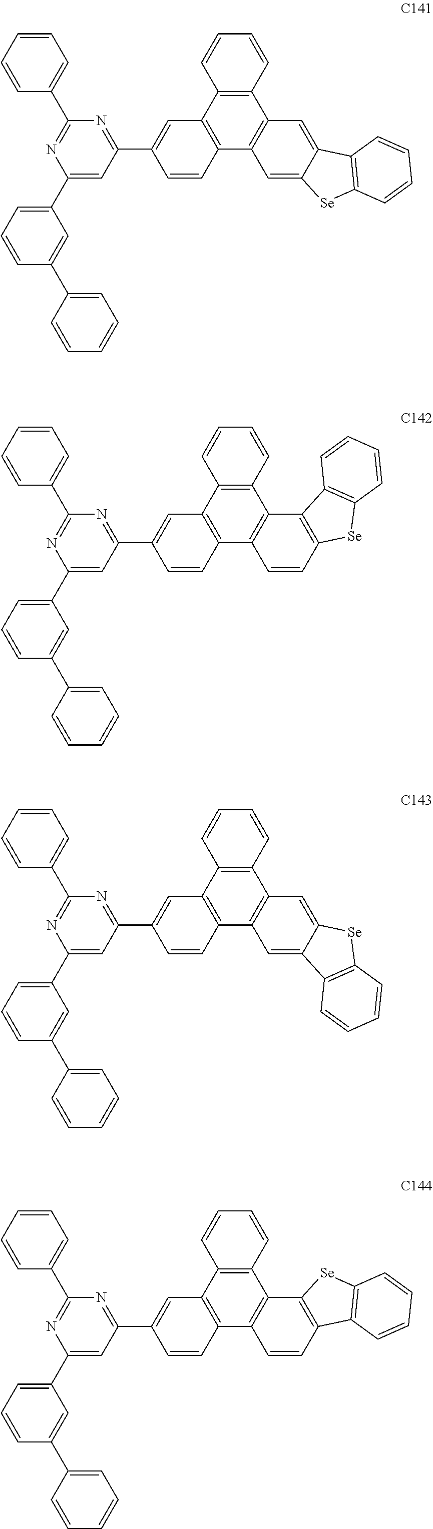 [SMILES: c1ccc(-c2cccc(-c3cc(-c4ccc5c(c4)c4ccccc4c4cc6c(cc54)[se]c4ccccc46)nc(-c4ccccc4)n3)c2)cc1 ; c1ccc(-c2cccc(-c3cc(-c4ccc5c(c4)c4ccccc4c4c5ccc5[se]c6ccccc6c54)nc(-c4ccccc4)n3)c2)cc1 ; c1ccc(-c2cccc(-c3cc(-c4ccc5c(c4)c4ccccc4c4cc6[se]c7ccccc7c6cc54)nc(-c4ccccc4)n3)c2)cc1 ; c1ccc(-c2cccc(-c3cc(-c4ccc5c(c4)c4ccccc4c4c5ccc5c6ccccc6[se]c54)nc(-c4ccccc4)n3)c2)cc1]

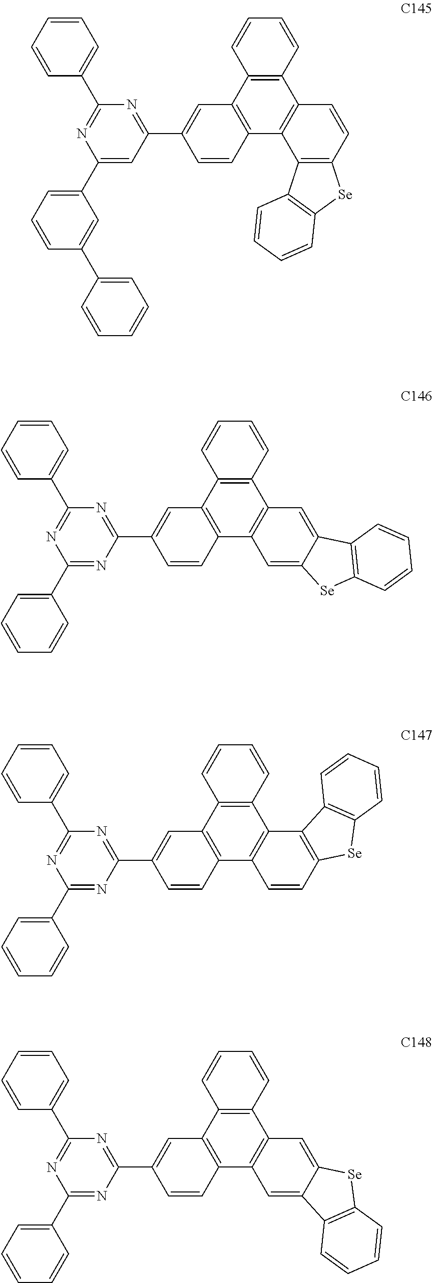 [SMILES: c1ccc(-c2cccc(-c3cc(-c4ccc5c(c4)c4ccccc4c4ccc6[se]c7ccccc7c6c45)nc(-c4ccccc4)n3)c2)cc1 ; c1ccc(-c2nc(-c3ccccc3)nc(-c3ccc4c(c3)c3ccccc3c3cc5c(cc43)[se]c3ccccc35)n2)cc1 ; c1ccc(-c2nc(-c3ccccc3)nc(-c3ccc4c(c3)c3ccccc3c3c4ccc4[se]c5ccccc5c43)n2)cc1 ; c1ccc(-c2nc(-c3ccccc3)nc(-c3ccc4c(c3)c3ccccc3c3cc5[se]c6ccccc6c5cc43)n2)cc1]

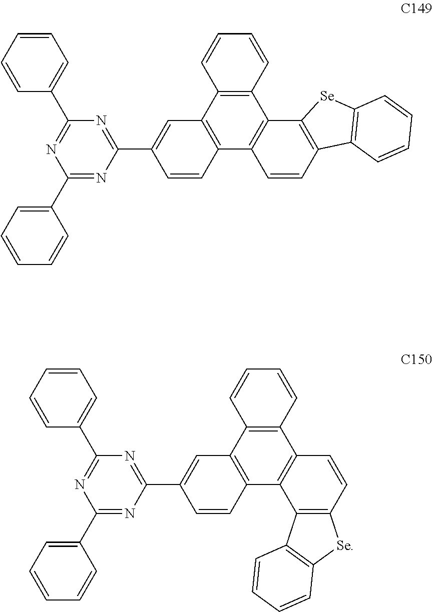

6. A red organic light emitting diode, comprising a first electrode, a second electrode, and an organic thin film layer between the first electrode and the second electrode, wherein the organic thin film layer is selected from the group consisting of a first light emitting layer, a first hole blocking layer, a first electron transport layer, and combinations thereof, wherein the first light emitting layer comprises a first host and a first guest, and wherein the organic thin film layer comprises the organic compound of claim 5.

7. The red organic light emitting diode of claim 6, wherein the first host comprises the organic compound as a first material, wherein the first hole blocking layer comprises the organic compound as a second material, and wherein the second material is the same as the first material.

8. The red organic light emitting diode of claim 7, further comprising a second light emitting layer and a second electron transport layer, wherein the second light emitting layer comprises a second host, wherein the second host comprises the organic compound as a third material, and wherein the third material is the same as the first material.

9. The red organic light emitting diode of claim 8, further comprising a second hole blocking layer.

10. The red organic light emitting diode of claim 9, wherein the second hole blocking layer comprises the organic compound as a fourth material, and wherein the fourth material is the same as the first material.

11. A red organic light emitting diode, comprising:
a substrate;
a first electrode on the substrate;
a first hole injection layer, a first hole transport layer, a first electron blocking layer and a first light emitting layer, formed on the first electrode, wherein the first light emitting layer comprises a first guest and a first host;
a second hole injection layer, a second hole transport layer and a second electron blocking layer on the first electron transport layer;
a second light emitting layer, on the second electron blocking layer, wherein the second light emitting layer comprises a second guest and a second host;
a second electrode on the second electron transport layer,
wherein the second host and the first host comprise the organic compound of claim 1 as a first organic compound.

12. The red organic light emitting diode of claim 11, wherein the first guest has an electroluminescence peak greater than about 600 nm.

13. The red organic light emitting diode of claim 11, wherein the second guest has an electroluminescence peak greater than about 600 nm.

14. The red organic light emitting diode of claim 11, wherein the first organic compound is selected from the group consisting of the following compounds:

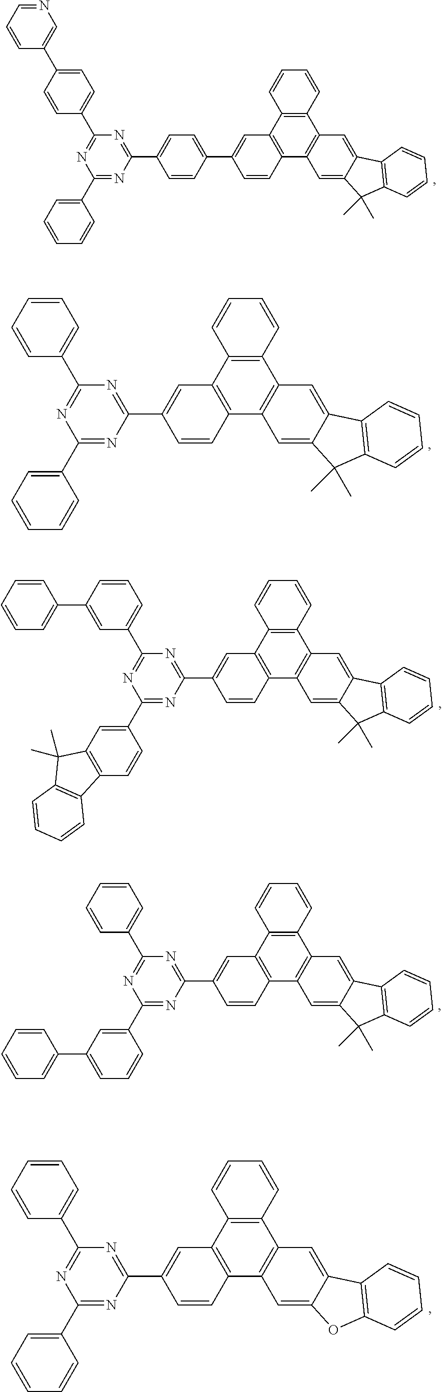 [SMILES: CC1(C)c2ccccc2-c2cc3c4ccccc4c4cc(-c5ccc(-c6nc(-c7ccccc7)nc(-c7ccc(-c8cccnc8)cc7)n6)cc5)ccc4c3cc21 ; CC1(C)c2ccccc2-c2cc3c4ccccc4c4cc(-c5nc(-c6ccccc6)nc(-c6ccccc6)n5)ccc4c3cc21 ; CC1(C)c2ccccc2-c2ccc(-c3nc(-c4cccc(-c5ccccc5)c4)nc(-c4ccc5c(c4)c4ccccc4c4cc6c(cc54)C(C)(C)c4ccccc4-6)n3)cc21 ; CC1(C)c2ccccc2-c2cc3c4ccccc4c4cc(-c5nc(-c6ccccc6)nc(-c6cccc(-c7ccccc7)c6)n5)ccc4c3cc21 ; c1ccc(-c2nc(-c3ccccc3)nc(-c3ccc4c(c3)c3ccccc3c3cc5c(cc43)oc3ccccc35)n2)cc1]

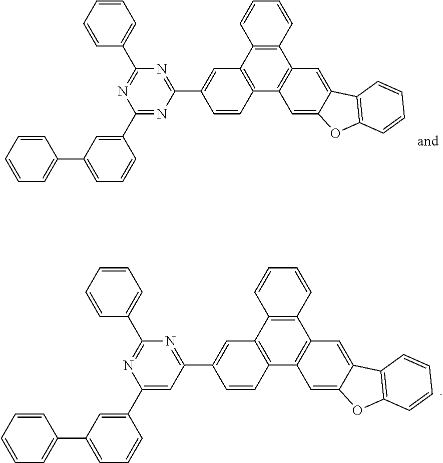 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3cc(-c4ccc5c(c4)c4ccccc4c4cc6c(cc54)oc4ccccc46)nc(-c4ccccc4)n3)c2)cc1]

15. The red organic light emitting diode of claim 11, wherein the second electron transport layer comprises a second organic compound.

16. The red organic light emitting diode of claim 15, wherein the second organic compound is the same as the first organic compound.

17. The red organic light emitting diode of claim 11, further comprising a hole blocking layer between the second light emitting layer and the second electron transport layer.

18. The red organic light emitting diode of claim 17, wherein the hole blocking layer comprises a third organic compound the same as the first organic compound.

19. The red organic light emitting diode of claim 14, wherein the second electron transport layer comprises a second organic compound, and wherein the second organic compound is the same as the first organic compound.

20. The red organic light emitting diode of claim 14, further comprising a hole blocking layer between the second light emitting layer and the second electron transport layer, wherein the hole blocking layer comprises a third organic compound the same as the first organic compound.
